(19) United States
(12) Patent Application Publication
(10) Pub. No.: US 2004/0018199 A1
(43) Pub. Date: Jan. 29, 2004

(54) Compositions and methods of use for an ephrin receptor

(76) Inventors: Raj Bandaru, Watertown, MA (US); Amitabha Chaudhuri, Madison, CT (US); Katherine Fries, North Brauford, CT (US); Michael E. Jeffers, Branford, CT (US); William J. LaRochelle, Madison, CT (US); Henri S. Lichenstein, Guilford, CT (US); Uriel M. Malyankar, Branford, CT (US); Chean Eng Ooi, Branford, CT (US); Sudhirdas K. Prayaga, O'Fallon, MO (US); Raymond J. Taupier JR., East Haven, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/449,569

(22) Filed: May 29, 2003

Related U.S. Application Data
(63) Continuation-in-part of application No. 09/689,486, filed on Oct. 12, 2000.
(63) Continuation-in-part of application No. 09/687,276, filed on Oct. 13, 2000.
(60) Provisional application No. 60/159,805, filed on Oct. 15, 1999.
(60) Provisional application No. 60/159,992, filed on Oct. 18, 1999.
(60) Provisional application No. 60/160,952, filed on Oct. 22, 1999.
(60) Provisional application No. 60/384,044, filed on May 29, 2002.
(60) Provisional application No. 60/402,171, filed on Aug. 9, 2002.
(60) Provisional application No. 60/412,527, filed on Sep. 20, 2002.

(51) Int. Cl. A61K 39/395

(52) U.S. Cl. 424/155.1

(57) ABSTRACT

In methods of using an ephrin receptor, such as for treating, preventing or delaying a proliferation-associated disorder such as cancer, steps are provided to administer to a subject a therapeutically effective amount of an antibody directed to an ephrin receptor polypeptide, variant or fragment thereof.

### RELATED APPLICATIONS

This application is a continuation-in-part of U.S. Ser. No. 09/689,486, filed Oct. 12, 2000 and U.S. Ser. No. 09/687,276 filed Oct. 13, 2000, both of which claim priority to U.S. Ser. No. 60/159,805, filed Oct. 15, 1999; U.S. Ser. No. 60/159,992, filed Oct. 18, 1999; U.S. Ser. No. 60/086,423, filed Oct. 18, 1999; and U.S. Ser. No.60/160,952 filed Oct. 22,1999. This application also claims priority from the Provisional applications U.S. Serial No. 60/384044 filed May 29, 2002, U.S. Ser. No. 60/402171 filed Aug. 9, 2002; and U.S. Ser. No. 60/412527 filed Sep. 20, 2002. The contents of these applications are incorporated herein by reference in their entireties.

### FIELD OF THE INVENTION

The invention generally relates to nucleic acids and polypeptides encoded therefrom and their methods of use. More specifically, the invention relates to nucleic acids encoding membrane bound and secreted polypeptides that are homologous to ephrin-type A receptors, as well as vectors, host cells, antibodies, and recombinant methods for producing these nucleic acids and polypeptides.

### BACKGROUND OF THE INVENTION

In 2002 about 550,000 Americans were expected to die of cancer (American Cancer Society: Cancer Facts and Figures 2002. Atlanta, Ga.: American Cancer Society, 2002). Cancer is the second leading cause of death in the US, exceeded only by heart disease.

Lung Cancer: Lung cancer is the second most common cancer among both men and women and is the leading cause of cancer death in both sexes. It is estimated that 169,400 new cases of lung cancer were diagnosed in 2002 and 154,900 people died, accounting for 28% of all cancer deaths in the United States (American Cancer Society: Cancer Facts and Figures 2002. Atlanta, Ga.: American Cancer Society, 2002).

Classification of lung carcinomas by histopathologic subtype provides important pathobiological information. There are two general classes of lung cancer: non-small cell lung cancer (NSCLC) and small cell lung cancer (SCLC). Treatment options and prognosis primarily depend on the stage and size of the tumor and the type of lung cancer. The therapeutic approach to treatment of lung carcinoma depends largely upon the histological type of tumor (NSCLC vs. SCLC), the stage of the tumor (based upon the characteristics of the primary tumor and the presence or absence of nodal and distant metastases), and potential for surgical removal.

NSCLC is divided into five subtypes, namely, squamous cell carcinoma, adenocarcinoma, large cell carcinoma, adenosquamous carcinoma, and undifferentiated carcinoma. NSCLCs are approximately equally divided between the two major histological subtypes, adenocarcinoma and squamous cell carcinoma (˜70% of total cases). The prevalence of adenocarcinoma is higher in women and this type of tumor is typically found in the peripheral tissue of the lung and has a predilection to disseminate. In contrast, squamous cell carcinoma (SCC) affects primarily men. Large cell NSCLC (˜10% of total cases) is the most aggressive and drug resistant NSCLC subtype.

At diagnosis, patients can be divided into three treatment groups based on the stage of the cancer. Stage 0, stage I, and stage II NSCLC can often be removed by surgery, including lobectomy or pneumonectomy. Radiation therapy may be used to treat patients who have other medical problems and cannot have surgery. NSCLC that has spread to nearby tissue or to lymph nodes can be treated with radiation therapy alone, radiation therapy combined with chemotherapy or surgery alone. Radiation therapy may be used to shrink the cancer and to relieve pain in patients who have NSCLC that has spread to other parts of the body.

Small cell carcinomas make up 20 to 25% of total lung carcinoma cases. Small cell carcinoma shows a strong correlation with cigarette smoking and is extremely rare in persons who have never smoked. In addition, these tumors are relatively more chemotherapy-sensitive, tend to be large central masses with almost guaranteed extensive mediatinal node involvement and frequent visceral metastasis at the time of diagnosis. For most patients with small cell lung cancer, current treatments do not cure the cancer.

Breast Cancer: Breast cancer is the most common form of cancer among women in the United States and is the second leading cause of cancer deaths after lung cancer. It is estimated that 205,000 new cases of breast cancer will be diagnosed in 2002 and 40,000 women will die from the disease (American Cancer Society: Cancer Facts and Figures 2002. Atlanta, Ga.: American Cancer Society, 2002). Mortality rates are highest in the very young (less than age 35) and the very old (greater than age 75). Perhaps as many as 55% of breast cancer cases can be explained by known risk factors such as age at menarche, age at first live birth, age at menopause, benign breast disease, and socioeconomic situation. An additional 10% of cases are associated with a positive family history.

Breast tumors may arise in the ductal epithelium (90%) or within the lobular epithelium (10%). Both ductal and lobular cancers can be further divided into those that have not penetrated the limiting basement membranes (noninfiltrating) and those that have (infiltrating). Of these, the infiltrating ductal carcinoma is the most common type, accounting for roughly 75% of breast carcinomas.

Early menarche, late menopause, and nulliparity are correlated with an increased risk of developing breast carcinoma, suggesting that prolonged exposure to cycling estrogen and progesterone levels contributes to the development of the disease (Keen and Davidson, Cancer 2003 97:825-33). Approximately 70-80% of all breast tumors express the estrogen receptor (ER) α protein and are therefore called ER positive. These tumors tend to grow more slowly, are better differentiated and are associated with a better overall prognosis. ER expression is an important indicator of potential response to endocrine therapy.

The majority of breast cancers are diagnosed as a result of an abnormal mammogram. Alternatively, a tumor can be discovered as a discrete, painless, and movable lump in the breast during a breast exam. At the time a lump can be felt, the tumor is typically less than 4 cm in diameter, however, involvement of the regional lymph nodes is already present in two-thirds of patients. A tissue biopsy is taken to obtain diagnostic material. Prognostic factors for breast cancer include ER expression, axillary lymph node status, tumor size and histologic grade and subtype.

Once the diagnosis of breast cancer is established, the choice of treatment depends upon the stage of disease. Breast cancers can be divided into the following categories: carcinoma in situ (CIS); early stage invasive breast cancer (stages I and II); locally advanced and inflammatory breast cancer (stage III); and metastatic breast cancer. Early stage invasive breast cancer is frequently treated by breast conserving therapy (BCT) or surgical removal of the tumor followed by radiation therapy (RT) to the entire breast. Some patients have contraindications to BCT that result in the recommendation of mastectomy.

Adjuvant systemic therapy with chemotherapy or hormone therapy after definitive local therapy represents a significant advance in the management of early breast cancer, significantly reducing the risk of both recurrence and death. All women with node-positive, and a significant proportion of those with node-negative disease (particularly those with hormone receptor negative tumors or those with tumors >1 cm in size) should receive adjuvant therapy. Adjuvant chemotherapy, often with two or more antineoplastic agents, has become the standard of care for women less than 50 years of age, regardless of their hormone receptor status. Premenopausal ER-positive women are usually also given adjuvant endocrine therapy, which may include tamoxifen, luteinizing hormone releasing hormone agonists such as goserelin, or ovariectomy.

### SUMMARY OF THE INVENTION

The present invention is based, in part, upon the discovery of nucleic acids encoding polypeptides having homology to an ephrin A8 receptor protein. Novel ephrin receptor protein (EPH-X) polynucleotide sequences, the EPH-X polypeptides encoded by these nucleic acid sequences, and antibodies that immunospecifically bind to these EPH-X polypeptides, and fragments, homologs, analogs, and derivatives thereof, are claimed in the invention.

In one aspect, the invention provides a method of treating, preventing, or delaying a cell proliferation-associated disorder by administering to a subject a therapeutically effective amount of an antibody that binds immunospecifically to an EPH-X polypeptide. The subject is a mammal, such as a human. In embodiments of the invention, the cell proliferation-associated disorder is lung cancer, breast cancer, or a cancer of the nervous system. In specific embodiments of the invention, the cell proliferation-associated disorder is lung cancer, metastatic lung cancer, lung adenocarcinoma, small cell lung cancer, squamous cell lung carcinoma, large cell carcinoma, adenosquamous carcinoma, undifferentiated lung carcinoma, breast cancer, infiltrating ductal carcinoma, metastatic breast cancer, or brain cancer.

In some embodiments of the invention, the antibody is a polyclonal antibody, a monoclonal antibody, or a humanized monoclonal antibody. The administration is by intravenous means. Alternatively, the administration is by parenteral means.

In one aspect, the invention provides a purified antibody that binds immunospecifically to an EPH-X polypeptide An EPH-X polypeptide includes: a) a polypeptide of SEQ ID NOS: 2, 4, 6, 8, 10, 12, 14, 16, 18, 20, 22, 24, 26, 28, 30, 32, 34, and 36; b) a mature form of a polypeptide of SEQ ID NOS: 2,4, 6, 8, 10, 12, 14, 16, 18, 20, 22, 24, 26, 28, 30, 32, 34, and 36; a variant EPH-X polypeptide, provided that the variant is no more than 15% divergent in sequence from the EPH-X polypeptide, and provided that the variant retains cellular proliferation modulatory activity; and d) a fragment of a EPH-X polypeptide, which fragment retains cellular proliferation modulatory activity. The antibody can be, e.g., a monoclonal or polyclonal antibody, and fragments, homologs, analogs, and derivatives thereof. In some embodiments, the antibody is a human monoclonal antibody. In some embodiments, the antibody is generated using a human antibody-producing mouse strain.

In some embodiments, the antibody is conjugated to a conjugation agent, such as a chemotherapic agent or a radiotherapic agent. Chemotherapic agents include diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins, momordica charantia inhibitor, curcin, crotin, Sapaonaria officinalis inhibitor, gelonin, mitogellin, restrictocin, phenomycin, enomycin, and a tricothecene. In other embodiments, the antibody is conjugated to an antibody conjugated to a toxin, such as saporin.

In another aspect, the invention provides an isolated EPH-X nucleic acid (SEQ ID NOs: 1, 3, 5, 7, 9, 11, 13, 15, 17, 19, 21, 23, 25, 27, 29, 31, 33, 35, as shown in Table 1), that encodes a EPH-X polypeptide, or a fragment, homolog, analog or derivative thereof. The nucleic acid can include, e.g., nucleic acid sequence encoding a polypeptide at least 85% identical to a polypeptide comprising the amino acid sequence of Table 1 (SEQ ID NOs: 2, 4, 6, 8, 10, 12, 14, 16, 18, 20, 22, 24, 26, 28, 30, 32, 34, 36). The nucleic acid can be, e.g., a genomic DNA fragment, or it can be a cDNA molecule.

Also included in the invention is a vector containing one or more of the nucleic acids described herein, and a cell containing the vectors or nucleic acids described herein.

The present invention is also directed to host cells transformed with a recombinant expression vector comprising any of the nucleic acid molecules described above.

In one aspect, the invention includes a pharmaceutical composition that includes a EPH-X nucleic acid and a pharmaceutically acceptable carrier or diluent. In a further aspect, the invention includes a substantially purified EPH-X polypeptide, e.g., any of the EPH-X polypeptides encoded by a EPH-X nucleic acid, and fragments, homologs, analogs, and derivatives thereof. The invention also includes a pharmaceutical composition that includes a EPH-X polypeptide and a pharmaceutically acceptable carrier or diluent.

In another aspect, the invention includes a method of preparing a pharmaceutical composition by combining at least one antibody effective in treating, preventing, or delaying a cell proliferation-associated disorder with a pharmaceutically acceptable carrier, where the antibody binds immunospecifically to an EPH-X polypeptide.

The invention also provides a method for determining the presence of or predisposition to a cell proliferation-associated disorder associated with altered levels of an EPH-X polypeptide in a first mammalian subject, by measuring the amount of the polypeptide in a sample from the first mammalian subject using an antibody that immunospecifically binds to the polypeptide; and comparing the amount of the polypeptide in the sample to the amount of the polypeptide present in a control sample from a second mammalian subject known not to have, or not to be predisposed to, the disorder; where an alteration in the level of the polypeptide in the first subject as compared to the control sample indicates the presence of or predisposition to the disorder.

The invention also provides a drug formulation for treating, preventing, or delaying a cell proliferation-associated disorder in a subject including a therapeutically effective amount of an antibody that immunospecifically binds an EPH-X polypeptide, and a formulation buffer.

The invention further provides a method of modulating the proliferation of a mammalian cell by contacting the cell with an antibody that immunospecifically binds to a polypeptide.

In another aspect, the invention provides a method of modulating blood vessel formation in a mammal, by contacting the mammal with an antibody that immunospecifically binds to an EPH-X polypeptide.

The invention also includes a pharmaceutical composition including EPH-X antibody and a pharmaceutically acceptable carrier or diluent. The present invention is also directed to isolated antibodies that bind to an epitope on an EPH-X polypeptide or a polypeptide encoded by any of the EPH-X nucleic acid molecules described herein.

The present invention is further directed to kits comprising antibodies that bind to a polypeptide encoded by any of the nucleic acid molecules described above and a negative control antibody.

The invention further provides a method for producing a EPH-X polypeptide. The method includes providing a cell containing a EPH-X nucleic acid, e.g., a vector that includes a EPH-X nucleic acid, and culturing the cell under conditions sufficient to express the EPH-X polypeptide encoded by the nucleic acid. The expressed EPH-X polypeptide is then recovered from the cell. Preferably, the cell produces little or no endogenous EPH-X polypeptide. The cell can be, e.g., a prokaryotic cell or eukaryotic cell.

The present invention provides a method of inducing an immune response in a mammal against a polypeptide encoded by any of the EPH-X nucleic acid molecules disclosed above by administering to the mammal an amount of the polypeptide sufficient to induce the immune response.

The present invention is also directed to methods of identifying a compound that binds to EPH-X polypeptide by contacting the EPH-X polypeptide with a compound and determining whether the compound binds to the EPH-X polypeptide.

The invention further provides methods of identifying a compound that modulates the activity of a EPH-X polypeptide by contacting EPH-X polypeptide with a compound and determining whether the EPH-X polypeptide activity is modified.

The present invention is also directed to compounds that modulate EPH-X polypeptide activity identified by contacting a EPH-X polypeptide with the compound and determining whether the compound modifies activity of the EPH-X polypeptide, binds to the EPH-X polypeptide, or binds to a nucleic acid molecule encoding a EPH-X polypeptide.

In another aspect, the invention provides a method of diagnosing a cell proliferation-associated disorder, such as cancer, e.g., lung cancer or breast cancer, in a subject. The method includes providing a protein sample from the subject and measuring the amount of EPH-X polypeptide in the subject sample. The amount of EPH-X in the subject sample is then compared to the amount of EPH-X polypeptide in a control protein sample. An alteration in the amount of EPH-X polypeptide in the subject protein sample relative to the amount of EPH-X polypeptide in the control protein sample indicates the subject has a cell proliferation-associated condition. A control sample is preferably taken from a matched individual, i.e., an individual of similar age, sex, or other general condition but who is not suspected of having a cell proliferation-associated condition. Alternatively, the control sample may be taken from the subject at a time when the subject is not suspected of having a cell proliferation-associated disorder. In some embodiments, the EPH-X polypeptide is detected using a EPH-X antibody.

In a further aspect, the invention includes a method of diagnosing a cell proliferation-associated disorder, such as cancer, in a subject. The method includes providing a nucleic acid sample, e.g., RNA or DNA, or both, from the subject and measuring the amount of the EPH-X nucleic acid in the subject nucleic acid sample. The amount of EPH-X nucleic acid sample in the subject nucleic acid is then compared to the amount of EPH-X nucleic acid in a control sample. An alteration in the amount of EPH-X nucleic acid in the sample relative to the amount of EPH-X in the control sample indicates the subject has a cell proliferation-associated disorder.

In another aspect, the invention includes a method of diagnosing a cell proliferation-associated disorder in a subject. The method includes providing a polypeptide sample from the subject and identifying at least a portion of the polypeptide of a EPH-X polypeptide in the subject polypeptide sample. The at least a portion of the polypeptide of a EPH-X polypeptide is identified using an EPH-X antibody. The EPH-X polypeptide of the subject sample is then compared to a EPH-X polypeptide of a control sample. An alteration in the EPH-X polypeptide in the sample relative to the EPH-X polypeptide in said control sample indicates the subject has a cell proliferation-associated disorder.

In a further aspect, the invention includes a method of diagnosing a cell proliferation-associated disorder in a subject. The method includes providing a nucleic acid sample from the subject and identifying at least a portion of the nucleotide sequence of a EPH-X nucleic acid in the subject nucleic acid sample. The EPH-X nucleotide sequence of the subject sample is then compared to a EPH-X nucleotide sequence of a control sample. An alteration in the EPH-X nucleotide sequence in the sample relative to the EPH-X nucleotide sequence in said control sample indicates the subject has a cell proliferation-associated disorder.

The method includes administering to a subject in which such treatment or prevention or delay is desired a EPH-X nucleic acid, a EPH-X polypeptide, or a EPH-X antibody in an amount sufficient to treat, prevent, or delay a cell proliferation-associated disorder in the subject.

The cell proliferation-associated disorders diagnosed, treated, prevented or delayed using the EPH-X nucleic acid molecules, polypeptides or antibodies can involve epithelial cells, mesenchymal and/or endothelial cells. The cell proliferation associated disorder can be lung cancer, metastatic lung cancer, lung adenocarcinoma, small cell lung cancer, squamous cell lung carcinoma, large cell carcinoma, adenosquamous carcinoma, undifferentiated lung carcinoma, breast cancer, infiltrating ductal carcinoma, or metastatic breast cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a photograph demonstrating transient expression of cgAL035703-S340-1C (CG54020-02, -03) in HEK 293 cells.

FIG. 2 is a photograph demonstrating stable expression of cgAL035703-S340-1C (CG54020-02, -03) in CHO-K1 cells.

FIG. 3 is a schematic illustration indicating Eph A8 Receptor Protein-Protein Interactions Identified by PathCalling.

### DETAILED DESCRIPTION OF THE INVENTION

Cancer is the second leading cause of death in the US, and lung cancer and breast cancer are the most common forms of cancer. Consequently, a therapeutic that can successfully treat lung and breast cancer has the beneficial effects of decreasing morbidity and mortality, while potentially saving the healthcare system millions of dollars in costs associated with invasive surgical procedures, radiation therapy, chemotherapy, and ancillary support services.

The present invention details the composition and use of an ephrin A8 receptor. Data in support of the invention indicate that Eprin A8 could be potentially used as a marker for diagnosis of lung, breast and brain cancers. Additionally, the antibodies against ephrin A8 receptor can be used as a therapeutic for the treatment of cancers including lung, breast and brain cancers.

Ephrin (Eph) receptors comprise the largest known family of receptor protein tyrosine kinases. They have been implicated in mediating developmental events, particularly in the nervous system. Receptors in the ephrin subfamily typically have a single kinase domain and an extracellular region containing a Cys-rich domain and two fibronectin type III repeats. Along with their ligands, called ephrins, they play important roles in neural development, angiogenesis, and vascular network assembly (9(4) Mol. Cells, 440-5 (Aug. 31, 1999)). The present invention details compositions of ephrin A8 receptors and their variants. Methods of using the invention as a diagnostic marker for cancer and the antibodies as a treatment for lung, breast and brain cancers are also included in the invention.

Ephrin receptors, together with their ephrin ligands, are important for a number of normal and pathologic processes. Specifically, these proteins are known to play important roles in neural development, angiogenesis, and vascular network assembly (Choi et al., Mol Cells 1999 9:440-5). Ephrin receptors typically have a single kinase domain and an extracellular region containing a Cys-rich domain and two fibronectin type III repeats. These receptors are divided into two groups based on the similarity of their extracellular domain sequences and their affinities for binding ephrin-A and ephrin-B ligands.

Ephrin receptors mediate contact-dependent cell interactions and, through this activity, play key roles in development of the nervous system as well as in angiogenesis. In the nervous system, ephrin receptors provide positional information by employing mechanisms that involve repulsion of migrating cells and growing axons (Frisen et al., EMBO J 1999 18:5159-65). Elevated expression of Eph receptors and their ligands is associated with tumors and associated tumor vasculature, suggesting that these proteins play critical roles in tumor angiogenesis and tumor growth (Cheng et al., Cytokine Growth Factor Rev 2002 13:75-85). In addition, ephrin ligands are known to be involved in determining cellular morphology and migration/invasion.

The ephrin receptor ligand ephrin-Al stimulates angiogenesis in vitro (Daniel et al., Kidney Int Suppl 1996 57:S73-81) and in vivo (Pandey et al., Science 1995 268:567-9). In addition, antisense targeting of Ephrin-A1 inhibits growth of cancer cells in vitro. Three of the ephrin A receptors have been directly implicated in cancer. Overexpression of the EphA1 receptor transforms 3T3 cells in vitro and induces their tumorigenicity in vivo (Maru et al., Oncogene 1990 5:445-7). Antibody targeting of the EphA2 receptor in inhibits tumor cell growth and branching in vitro (Carles-Kinch et al., Cancer Res 2002 62:2840-7). Furthermore, soluble EphA2 and EphA3 receptors inhibit angiogenesis and tumor growth in vivo (Brantley et al., Oncogene 2002 21:7011-26).

The ephrin type-A receptor 8, EphA8 or Eek, is a Type I membrane-bound protein that serves as a receptor for members of the ephrin-A family. Specifically, the EphA8 receptor has been shown to interact with ephrin-A1 to -A5 ligands (Park and Sanchez, Oncogene 1997 14:533-42; Choi et al., Mol Cells 1999 9:440-5). Its catalytic activity is as a protein tyrosine kinase, phosphorylating tyrosine in appropriate target proteins. EphA8 has also been shown to enhance cell attachment and migration in a kinase-independent manner via localization of the p110γ PI 3-kinase to the plasma membrane, thereby allowing access to lipid substrates to enable the signals required for integrin-mediated cell adhesion (Gu and Park, Mol Cell Biol 2001 21:4579-97). The mouse EphA8 gene is not essential; EphA8 knock-out mice possess minor aberrant axonal projections but are otherwise normal (Park et al., EMBO J 1997 16:3106-14). Because members of the Ephrin A receptor gene family are involved in cell migration, angiogenesis and/or invasion, the CG54020 gene might be a potential target for therapy based upon the inhibition of tumor metastasis and angiogenesis.

Included within the invention are EPH-X nucleic acids, isolated nucleic acids that encode EPH-X polypeptide or a portion thereof, EPH-X polypeptides, vectors containing these nucleic acids, host cells transformed with the EPH-X nucleic acids, anti-EPH-X antibodies, and pharmaceutical compositions. Also disclosed are methods of making EPH-X polypeptides, as well as methods of screening, diagnosing, treating conditions using these compounds, and methods of screening compounds that modulate EPH-X polypeptide activity. Table 1 provides a summary of the EPH-X nucleic acids and their encoded polypeptides.

TABLE 1

| | | SEQ | SEQ | |
|---|---|---|---|---|
| | | ID | ID | |
| EPH-X | | NO | NO | |
| Assign- | Internal | (nucleic | (amino | |
| ment | Identification | acid) | acid) | Homology |
| EPH1a | CG54020-01 | 1 | 2 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1b | CG54020-02 | 3 | 4 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1c | 248209335 | 5 | 6 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1d | 248209389 | 7 | 8 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1e | 252417844 | 9 | 10 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) |
| | | | | (HEK3) - Homo sapiens |
| EPH1f | 252417852 | 11 | 12 | Ephrin type-A receptor 8 |
| | | | | precursor (EC 2.7.1.112) |
| | | | | (Tyrosine-protein kinase |
| | | | | receptor EEK) (EPH-and |
| | | | | ELK-relate kinase) (HEK3) - |
| | | | | Home sapiens |
| EPH1g | 252417872 | 13 | 14 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1h | 252417876 | 15 | 16 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1i | 248213633 | 17 | 18 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1j | 248213637 | 19 | 20 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1k | 248209276 | 21 | 22 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1l | 248213660 | 23 | 24 | Ephrin type-A receptor 8 |
| | | | | precursor (EC 2.7.1.112) |
| | | | | (Tyrosine-protein kinase |
| | | | | EEK) (EPH-and |
| | | | | ELK-related kinase) (HEK3) - |
| | | | | Homo sapiens |
| EPH1m | 248213680 | 25 | 26 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1n | 248213688 | 27 | 28 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1o | 248209393 | 29 | 30 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1p | CG54020-03 | 31 | 32 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1q | CG54020-04 | 33 | 34 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |
| EPH1r | CG54020-05 | 35 | 36 | Ephrin type-A receptor 8 |
| | | | | precursor (EC 2.7.1.112) |
| | | | | (Tyrosine-protein kinase |
| | | | | receptor EEK) (EPH-and |
| | | | | ELK-related kinase) (HEK3) - |
| | | | | Homo sapiens |

By “cellular proliferation modulatory activity” is meant any biological, biochemical, or chemical action that increases or decreases the proliferation and/or differentiation of a eukaryotic cell, either in vivo, ex vivo, or in vitro, and includes inhibition and stimulation of apoptosis.

By “detectable entity” is meant any compound, molecule, biological material, or other composition of matter capable of being detected using means known to one of skill in the art, including fluorescent, luminescent, bioluminescent, biochemical and radioisotopic detection means.

### EXAMPLE 1

The ephrin A8 receptors and their variants were analyzed, and the nucleotide and encoded polypeptide sequences are shown in Table 1A.

TABLE 1A
EPH1 Sequence Analysis

| | | |
|---|---|---|
| EPH1a, CG54020-01 | SEQ ID NO: 1 | 3018 bp |
| DNA Sequence | ORF Start: ATG at 1 | ORF Stop: end of sequence |

ATGGCCCCCGCCCGGGGCCGCCTGCCCCCTGCGCTCTGGGTCGTCACGGCCGCGGCGGCGGCGGCCAC
CTGCGTGTCCGCGGCGCGCGGCGAAGTGAATTTGCTGGACACGTCGACCATCCACGGGGACTGGGGCT
GGCTCACGTATCCGGCTCATGGGTGGGACTCCATCAACGAGGTGGACGAGTCCTTCCAGCCCATCCAC
ACGTACCAGGTTTGCAACGTCATGAGCCCCAACCAGAACAACTGGCTGCGCACGAGCTGGGTCCCCCG
AGACGGCGCCCGGCGCGTCTATGCTGAGATCAAGTTTACCCTGCGCGACTGCAACAGCATGCCTGGTG
TGCTGGGCACCTGCAAGGAGACCTTCAACCTCTACTACCTGGAGTCGGACCGCGACCTGGGGGCCAGC
ACACAAGAAAGCCAGTTCCTCAAAATCGACACCATTGCGGCCGACGAGAGCTTCACAGGTGCCGACCT
TGGTGTGCGGCGTCTCAAGCTCAACACGGAGGTGCGCAGTGTGGGTCCCCTCAGCAAGCGCGGCTTCT
ACCTGGCCTTCCAGGACATAGGTGCCTGCCTGGCCATCCTCTCTCTCCGCATCTACTATAAGAAGTGC
CCTGCCATGGTGCGCAATCTGGCTCCCTTCTCGGAGGCAGTGACGGGGGCCGACTCGTCCTCACTGGT
GGAGGTGAGGGGCCAGTGCGTGCGGCACTCAGAGGAGCGGGACACACCCAAGATGTACTGCAGCGCGG
AGGGCGAGTGGCTCGTGCCCATCGGCAAATGCGTGTGCAGTGCCGCCTACGAGGAGCGGCGGGATGCC
TGTGTGGCCTGTGAGCTGGGCTTCTACAAGTCAGCCCCTGGGGACCAGCTGTGTGCCCGCTGCCCTCC
CCACAGCCACTCCGCAGCTCCAGCCGCCCAAGCCTGCCACTGTGACCTCAGCTACTACCGTGCAGCCC
TGGACCCGCCGTCCTCAGCCTGCACCCGGCCACCCTCGGCACCAGTGAACCTGATCTCCAGTGTGAAT
GGGACATCAGTGACTCTGGAGTGGGCCCCTCCCCTGGACCCAGGTGGCCGCAGTGACATCACCTACAA
TGCCGTGTGCCGCCGCTGCCCCTGGGCACTGAGCCGCTGCGAGGCATGTGGGAGCGGCACCCGCTTTG
TGCCCCAGCAGACAAGCCTGGTGCAGGCCAGCCTGCTGGTGGCCAACCTGCTGGCCCACATGAACTAC
TCCTTCTGGATCGAGGCCGTCAATGGCGTGTCCGACCTGAGCCCCGAGCCCCGCCGGGCCGCTGTGGT
CAACATCACCACGAACCAGGCAGCCCCGTCCCAGGTGGTGGTGATCCGTCAAGAGCGGGCGGGGCAGA
CCAGCGTCTCGCTGCTGTGGCAGGAGCCCGAGCAGCCGAACGGCATCATCCTGGAGTATGAGATCAAG
TACTACGAGAAGGACAAGGAGATGCAGAGCTACTCCACCCTCAAGGCCGTCACCACCAGAGCCACCGT
CTCCGGCCTCAAGCCGGGCACCCGCTACGTGTTCCAGGTCCGAGCCCGCACCTCAGCAGGCTGTGGCC
GCTTCAGCCAGGCCATCGAGGTGGAGACCGGGAAACCCCGGCCCCGCTATGACACCAGGACCATTGTC
TGGATCTGCCTGACGCTCATCACGGGCCTGGTGGTGCTTCTGCTCCTGCTCATCTGCAAGAAGAGGCA
CTGTGGCTACAGCAAGGCCTTCCAGGACTCGGACGAGGAGAAGATGCACTATCAGAATGGACAGGCAC
CCCCACCTGTCTTCCTGCCTCTGCATCACCCCCCGGGAAAGCTCCCAGAGCCCCAGTTCTATGCGGAA
CCCCACACCTACGAGGAGCCAGGCCGGGCGGGCCGCAGTTTCACTCGGGAGATCGAGGCCTCTAGGAT
CCACATCGAGAAAATCATCGGCTCTGGAGACTCCGGGGAAGTCTGCTACGGGAGGCTGCGGGTGCCAG
GGCAGCGGGATGTGCCCGTGGCCATCAAGGCCCTCAAAGCCGGCTACACGGAGAGACAGAGGCGGGAC
TTCCTGAGCGAGGCGTCCATCATGGGGCAATTCGACCATCCCAACATCATCCGCCTCGAGGGTGTCGT
CACCCGTGGCCGCCTGGCAATGATTGTGACTGAGTACATGGAGAACGGCTCTCTGGACACCTTCCTGA
GGACCCACGACGGGCAGTTCACCATCATGCAGCTGGTGGGCATGCTGAGAGGAGTGGGTGCCGGCATG
CGCTACCTCTCAGACCTGGGCTATGTCCACCGAGACCTGGCCGCCCGCAACGTCCTGGTTGACAGCAA
CCTGGTCTGCAAGGTGTCTGACTTCGGGCTCTCACGGGTGCTGGAGGACGACCCGGATGCTGCCTACA
CCACCACGGGCGGGAAGATCCCCATCCGCTGGACGGCCCCAGAGGCCATCGCCTTCCGCACCTTCTCC
TCGGCCAGCGACGTGTGGAGCTTCGGCGTGGTCATGTGGGAGGTGCTGGCCTATGGGGAGCGGCCCTA
CTGGAACATGACCAACCGGGATGTGATCAGCTCTGTGGAGGAGGGGTACCGCCTGCCCGCACCCATGG
CCTGCCCCCACGCCCTGCACCAGCTCATGCTCGACTGTTGGCACAAGGACCGGGCGCAGCGGCCTCGC
TTCTCCCAGATTGTCAGTGTCCTCGATGCGCTCATCCGCAGCCCTGAGAGTCTCAGGGCCACCGCCAC
AGTCAGCAGGTGCCCACCCCCTGCCTTCGTCCGGAGCTGCTTTGACCTCCGAGGGGGCAGCGGTGGCG
GTGGGGGCCTCACCGTGGGGGACTGGCTGGACTCCATCCGCATGGGCCGGTACCGAGACCACTTCGCT
GCGGGCGGATACTCCTCTCTGGGCATGGTGCTACGCATGAACGCCCAGGACGTGCGCGCCCTGGGCAT
CACCCTCATGGGCCACCAGAAGAAGATCCTGGGCAGCATTCAGACCATGCGGGCCCAGCTGACCAGCA
CCCAGGGGCCCCGCCGGCACCTCTGA

| | | | |
|---|---|---|---|
| EPH1a, CG54020-01 | SEQ ID NO: 2 | 1005 aa | MW at 11 1001.3kD |
| Protein Sequence | | | |

MAPARGRLPPALWVVTAAAAAATCVSAARGEVNLLDTSTIHGDWGWLTYPAHGWDSINEVDESFQPIH
TYQVCNVMSPNQNNWLRTSWVPRDGARRVYAEIKFTLRDCNSMPGVLGTCKETFNLYYLESDRDLGAS
TQESQFLKIDTIAADESFTGADLGVRRLKLNTEVRSVGPLSKRGFYLAFQDIGACLAILSLRIYYKKC
PAMVRNLAAFSEAVTGADSSSLVEVRGQCVRHSEERDTPKMYCSAEGEWLVPIGKCVCSAGYEERRDA
CVACELGFYKSAPGDQLCARCPPHSHSAAPAAQACHCDLSYYRAALDPPSSACTRPPSAPVNLISSVN
GTSVTLEWAPPLDPGGRSDITYNAVCRRCPWALSRCEACGSGTRFVPQQTSLVQASLLVANLLAHMNY
SFWIEAVNGVSDLSPEPRRAAVVNITTNQAAPSQVVVIRQERAGQTSVSLLWQEPEQPNGIILEYEIK
YYEKDKEMQSYSTLKAVTTRATVSGLKPGTRYVFQVRARTSAGCGRFSQAMEVETGKPRPRYDTRTIV
WICLTLITGLVVLLLLLICKKRHCGYSKAFQDSDEEKMHYQNGQAPPPVFLPLHHPPGKLPEPQFYAE
PHTYEEPGRAGRSFTREIEASRIHIEKIIGSGDSGEVCYGRLRVPGQRDVPVAIKALKAGYTERQRRD
FLSEASIMGQFDHPNIIRLEGVVTRGRLAMIVTEYMENGSLDTFLRTHDGQFTIMQLVGMLRGVGAGM
RYLSDLGYVHRDLAARNVLVDSNLVCKVSDFGLSRVLEDDPDAAYTTTGGKIPIRWTAPEAIAFRTFS
SASDVWSFGVVMWEVLAYGERPYWNMTNRDVISSVEEGYRLPAPMGCPHALHQLMLDCWHKDRAQRPR
FSQIVSVLDALIRSPESLRATATVSRCPPPAFVRSCFDLRGGSGGGGGLTVGDWLDSIRMGRYRDHFA
AGGYSSLGMVLRMNAQDVRALGITLMGHQKKILGSIQTMRAQLTSTQGPRRHL

| | | |
|---|---|---|
| EPH1b, CG54020-02 | SEQ ID NO: 3 | 1545 bp |
| DNA Sequence | ORF Start: at 1 | ORF Stop: end of sequence |

GCGCGCGGCGAAGTGAATTTGCTGGACACGTCGACCATCCACGGGGACTGGGGCTGGCTCACGTATCC
GGCTCATGGGTGGGACTCCATCAACGAGGTGGACGAGTCCTTCCAGCCCATCCACACGTACCAGGTTT
GCAACGTCATGAGCCCCAACCAGAACAACTGGCTGCGCACGAGCTGGGTCCCCCGAGACGGCGCCCGG
CGCGTCTATGCTGAGATCAAGTTTACCCTGCGCGACTGCAACAGCATGCCTGGTGTGCTGGGCACCTG
CAAGGAGACCTTCAACCTCTACTACCTGGAGTCGGACCGCGACCTGGGGGCCAGCACACAAGAAAGCC
AGTTCCTCAAAATCGACACCATTGCGGCCGACGAGAGCTTCACAGGTGCCGACCTTGGTGTGCGGCGT
CTCAAGCTCAACACGGAGGTGCGCAGTGTGGGTCCCCTCAGCAAGCGCGGCTTCTACCTGGCCTTCCA
GGACATAGGTGCCTGCCTGGCCATCCTCTCTCTCCGCATCTACTATAAGAAGTGCCCTGCCATGGTGC
GCAATCTGGCTGCCTTCTCGGAGGCAGTGACGGGGGCCGACTCGTCCTCACTGGTGGAGGTGAGGGGC
CAGTGCGTGCGGCACTCAGAGGAGCGGGACACACCCAAGATGTACTGCAGCGCGGAGGGCGAGTGGCT
CGTGCCCATCGGCAAATGCGTGTGCAGTGCCGGCTACGAGGAGCGGCGGGATGCCTGTGTGGCCTGTG
AGCTGGGCTTCTACAAGTCAGCCCCTGGGGACCAGCTGTGTGCCCGCTGCCCTCCCCACAGCCACTCC
GCAGCTCCAGCCGCCCAAGCCTGCCACTGTGACCTCAGCTACTACCGTGCAGCCCTGGACCCGCCGTC
CTCAGCCTGCACCCGGCCACCCTCGGCACCAGTGAACCTGATCTCCAGTGTGAATGGGACATCAGTGA
CTCTGGAGTGGGCCCCTCCCCTGGACCCAGGTGGCCGCAGTGACATCACCTACAATGCCGTGTGCCGC
CGCTGCCCCTGGGCACTGAGCCGCTGCGAGGCATGTGGGAGCGGCACCCGCTTTGTGCCCCAGCAGAC
AAGCCTGGTGCAGGCCAGCCTGCTGGTGGCCAACCTGCTGGCCCACATGAACTACTCCTTCTGGATCG
AGGCCGTCAATGGCGTGTCCGACCTGAGCCCCGAGCCCCGCCGGGCCGCTGTGGTCAACATCACCACG
AACCAGGCAGCCCCGTCCCAGGTGGTGGTGATCCGTCAAGAGCGGGCGGGGCAGACCAGCGTCTCGCT
GCTGTGGCAGGAGCCCGAGCAGCCGAACGGCATCATCCTGGAGTATGAGATCAAGTACTACGAGAAGG
ACAAGGAGATGCAGAGCTACTCCACCCTCAAGGCCGTCACCACCAGAGCCACCGTCTCCGGCCTCAAG
CCGGGCACCCGCTACGTGTTCCAGGTCCGAGCCCGCACCTCAGCAGGCTGTGGCCGCTTCAGCCAGGC
CATGGAGGTGGAGACCGGGAAACCCCGGCCCCGCTATGACACCAGGACC

| | | | |
|---|---|---|---|
| EPH1b, CG54020-02 | SEQ ID NO: 4 | 515 aa | MW at 56842.5kD |
| Protein Sequence | | | |

ARGEVNLLDTSTIHGDWGWLTYPAHGWDSINEVDESFQPIHTYQVCNVMSPNQNNWLRTSWVPRDGAR
RVYAEIKFTLRDCNSMPGVLGTCKETFNLYYLESDRDLGASTQESQFLKIDTIAADESFTGADLGVRR
LKLNTEVRSVGPLSKRGFYLAFQDIGACLAILSLRIYYKKCPAMVRNLAAFSEAVTGADSSSLVEVRG
QCVRHSEERDTPKMYCSAEGEWLVPIGKCVCSAGYEERRDACVACELGFYKSAPGDQLCARCPPHSHS
AAPAAQACHCDLSYYRAALDPPSSACTRPPSAPVNLISSVNGTSVTLEWAPPLDPGGRSDITYNAVCR
RCPWALSRCEACGSGTRFVPQQTSLVQASLLVANLLAHMNYSFWIEAVNGVSDLSPEPRRAAVVNITT
NQAAPSQVVVIRQERAGQTSVSLLWQEPEQPNGIILEYEIKYYEKDKEMQSYSTLKAVTTRATVSGLK
PGTRYVFQVRARTSAGCGRFSQAMEVETGKPRPRYDTRT

| | | |
|---|---|---|
| EPH1c, 248209335 | SEQ ID NO:5 | 1135 bp |
| DNA Sequence | ORF Start: at 2 | ORF Stop: end of sequence |

CACCAGATCTATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGACCCACGACGGGCAGTTCACCATCATGCAGCTGGTGGGCATGCTGAGAGGAGTG
GGTGCCGGCATGCGCTACCTCTCAGACCTGGGCTATGTCCACCGAGACCTGGCCGCCCGCAACGTCCT
GCTTGACAGCAACCTGGTCTGCAAGGTGTCTGACTTCGGGCTCTCACGGGTGCTGGAGGACGACCCGG
ATGCTGCCTACACCACCACGGGCGGGAAGATCCCCATCCGCTGGACGGCCCCAGAGGCCATCGCCTTC
CGCACCTTCTCCTCGGCCAGCGACGTGTGGAGCTTCGGCGTGGTCATGTGGGAGGTGCTGGCCTATGG
GGAGCGGCCCTACTGGAACATGACCAACCGGGATGTCATCAGCTCTGTGGAGGAGGGGTACCGCCTGC
CCGCACCCATGGGCTGCCCCCACGCCCTGCACCAGCTCATGCTCGACTGTTGGCACAAGGACCGGGCG
CAGCGGCCTCGCTTCTCCCAGATTGTCAGTGTCCTCGATGCGCTCATCCGCAGCCCTGAGAGTCTCAG
GGCCACCGCCACAGTCAGCAGGTGCCCACCCCCTGCCTTCGTCCGGAGCTGCTTTGACCTCCGAGGGG
GCAGCGGTGGCGGTGGGGGCCTCACCGTGGGGGACTGGCTGGACTCCATCCGCATGGGCCGGTACCGA
GACCACTTCGCTGCGGGCGGATACTCCTCTCTGGGCATGGTGCTACGCATGAACGCCCAGGACGTGCG
CGCCCTGGGCATCACCCTCATGGGCCACCAGAAGAAGATCCTGGGCAGCATTCAGACCATGCGGGCCC
AGCTGACCAGCACCCAGGGGCCCCGCCGGCACCTCTGAAAGCTTGGC

| | | | |
|---|---|---|---|
| EPH1c, 248209335 | SEQ ID NO: 6 | 374 aa | MW at 41422.1kD |
| Protein Sequence | | | |

TRSIHIEKIIGSGDSGEVCYGRLRVPGQRDVPVAIKALKAGYTERQRRDFLSEASIMGQFDHPNIIRL
EGVVTRGRLAMIVTEYMENGSLDTFLRTHDGQFTIMQLVGMLRGVGAGMRYLSDLGYVHRDLAARNVL
VDSNLVCKVSDFGLSRVLEDDPDAAYTTTGGKIPIRWTAPEAIAFRTFSSASDVWSFGVVMWEVLAYG
ERPYWNMTNRDVISSVEEGYRLPAPMGCPHALHQLMLDCWHKDRAQRPRFSQIVSVLDALIRSPESLR
ATATVSRCPPPAFVRSCFDLRGGSGGGGGLTVGDWLDSIRMGRYRDHFAAGGYSSLGMVLRMNAQDVR
ALGITLMGHQKKILGSIQTMRAQLTSTQGPRRHL

| | | |
|---|---|---|
| EPH1d, 248209389 | SEQ ID NO: 7 | 925 bp |
| DNA Sequence | ORF Start: at 2 | ORF Stop: end of sequence |

CACCAGATCTATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGGGCGGGAAGATCCCCATCCGCTGGACGGCCCCAGAGGCCATCGCCTTCCGCACC
TTCTCCTCGGCCAGCGACGTGTGGAGCTTCGGCGTGGTCATGTGGGAGGTGCTGGCCTATGGGGAGCG
GCCCTACTGGAACATGACCAACCGGGATGTCATCAGCTCTGTGGAGGAGGGGTACCGCCTGCCCGCAC
CCATGGGCTGCCCCCACGCCCTGCACCAGCTCATGCTCGACTGTTGGCACAAGGACCGGGCGCAGCGG
CCTCGCTTCTCCCAGATTGTCAGTGTCCTCGATGCGCTCATCCGCAGCCCTGAGAGTCTCAGGGCCAC
CGCCACAGTCAGCAGGTGCCCACCCCCTGCCTTCGTCCGGAGCTGCTTTGACCTCCGAGGGGGCAGCG
GTGGCGGTGGGGGCCTCACCGTGGGGGACTGGCTGGACTCCATCCGCATGGGCCGGTACCGAGACCAC
TTCGCTGCGGGCGGATACTCCTCTCTGGGCATGGTGCTACGCATGAACGCCCAGGACGTGCGCGCCCT
GGGCATCGCCCTCATGGGCCACCAGAAGAAGATCCTGGGCAGCATTCAGACCATGCGGGCCCAGCTGA
CCAGCACCCAGGGGCCCCGCCGGCACCTCTGAAAGCTTGGC

| | | | |
|---|---|---|---|
| EPH1d, 248209389 | SEQ ID NO: 8 | 304 aa | MW at 33763.5kD |
| Protein Sequence | | | |

TRSIHIEKIIGSGDSGEVCYGRLRVPGQRDVPVAIKALKAGYTERQRRDFLSEASIMGQFDHPNIIRL
EGVVTRGRLAMIVTEYMENGSLDTFLRGGKIPIRWTAPEAIAFRTFSSASDVWSFGVVMWEVLAYGER
PYWNMTNRDVISSVEEGYRLPAPMGCPHALHQLMLDCWHKDRAQRPRFSQIVSVLDALIRSPESLRAT
ATVSRCPPPAFVRSCFDLRGGSGGGGGLTVGDWLDSIRMGRYRDHFAAGGYSSLGMVLRMNAQDVRAL
GIALMGHQKKILGSIQTMRAQLTSTQGPRRHL

| | | |
|---|---|---|
| EPH1e, 252417844 | SEQ ID NO:9 | 925 bp |
| DNA Sequence | Start: at 2 | ORF Stop: end of sequence |

CACCAGATCTATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTAGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGGGCGGGAAGATCCCCATCCGCTGGACGGCCCCAGAGGCCATCGCCTTCCGCACC
TTCTCCTCGGCCAGCGACGTGTGGAGCTTCGGCGTGGTCATGTGGGAGGTGCTGGCCTATGGGGAGCG
GCCCTACTGGAACATGACCAACCGGGATGTCATCAGCTCTGTGGAGGAGGGGTACCGCCTGCCCGCAC
CCATGGGCTGCCCCCACGCCCTGCACCAGCTCATGCTCGACTGTTGGCACAAGGACCGGGCGCAGCGG
CCTCGCTTCTCCCAGATTGTCAGTGTCCTCGATGCGCTCATCCGCAGCCCTGAGAGTCTCAGGGCCAC
CGCCACAGTCAGCAGGTGCCCACCCCCTGCCTTCGTCCGGAGCTGCTTTGACCTCCGAGGGGGCAGCG
GTGGCGGTGGGGGCCTCACCGTGGGGGACTGGCTGGACTCCATCCGCATGGGCCGGTACCGAGACCAC
TTCGCTGCGGGCGGATACTCCTCTCTGGGCATGGTGCTACGCATGAACGCCCAGGACGTGCGCGCCCT
GGGCATCACCCTCATGGGCCACCAGAAGAAGATCCTGGGCAGCATTCAGACCATGCGGGCCCAGCTGA
CCAGCACCCAGGGGCCCCGCCGGCACCTCTGAAAGCTTGGC

| | | | |
|---|---|---|---|
| EPH1e, 252417844 | SEQ ID NO: 10 | 304 aa | MW at 33759.5kD |
| Protein Sequence | | | |

TRSIHIEKIIGSGDSGEVCYGRLRVPGQRDVPVAIKALKAGYTERQRRDFLSEASIMGQLDHPNIIRL
EGVVTRGRLAMIVTEYMENGSLDTFLRGGKIPIRWTAPEAIAFRTFSSASDVWSFGVVMWEVLAYGER
PYWMMTNRDVISSVEEGYRLPAPMGCPHALHQLMLDCWHKDRAQRPRFSQIVSVLDALIRSPESLRAT
ATVSRCPPPAFVRSCFDLRGGSGGGGGLTVGDWLDSIRMGRYRDHFAAGGYSSLGMVLRMNAQDVRAL
GITLMGHQKKILGSIQTMRAQLTSTQGPRRHL

| | | |
|---|---|---|
| EPH1f, 252417852 | SEQ ID NO: 11 | 925 bp |
| DNA Sequence | ORF Start: at 2 | ORF Stop: end of sequence |

CACCAGATCTATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGTCTCTCTGG
ACACCTTCCTGAGGGGCGGGAAGATCCCCATCCGCTGGACGGCCCCAGAGGCCATCGCCTTCCGCACC
TTCTCCTCGGCCAGCGACGTGTGGAGCTTCGGCGTGGTCATGTGGGAGGTGCTGGCCTATGGGGAGCG
GCCCTACTGGAACATGACCAACCGGGATGTCATCAGCTCTGTGGAGGAGGGGTACCGCCTGCCCGCAC
CCATGGGCTGCCCCCACGCCCTGCACCAGCTCATGCTCGACTGTTGGCACAAGGACCGGGCGCAGCGG
CCTCGCTTCTCCCAGATTGTCAGTGTCCTCGATGCGCTCATCCGCAGCCCTGAGAGTCTCAGGGCCAC
CGCCACAGTCAGCAGGTGCCCACCCCCTGCCTTCGTCCGGAGCTGCTTTGACCTCCGAGGGGGCAGCG
GTGGCGGTGGGGGCCTCACCGTGGGGGACTGGCTGGACTCCATCCGCATGGGCCGGTACCGAGACCAC
TTCGCTGCGGGCGGATACTCCTCTCTGGGCATGGTGCTACGCATGAACGCCCAGGACGTGCGCGCCCT
GGGCATCACCCTCATGGGCCACCAGAAGAAGATCCTGGGCAGCATTCAGACCATGCGGGCCCAGCTGA
CCAGCACCCAGGGGCCCCGCCGGCACCTCTGAAAGCTTGGC

| | | | |
|---|---|---|---|
| EPH1f, 252417852 | SEQ ID NO: 12 | 304 aa | MW at 33835.6kD |
| Protein Sequence | | | |

TRSIHIEKIIGSGDSGEVCYGRLRVPGQRDVPVAIKALKAGYTERQRRDFLSEASIMGQFDHPNIIRL
EGVVTRGRLAMIVTEYMENVSLDTFLRGGKIPIRWTAPEAIAFRTFSSASDVWSFGVVMWEVLAYGER
PYWNMTNRDVISSVEEGYRLPAPMGCPHALHQLMLDCWHKDRAQRPRFSQIVSVLDALIRSPESLRAT
ATVSRCPPPAFVRSCFDLRGGSGGGGGLTVGDWLDSIRMGRYRDHFAAGGYSSLGMVLRMNAQDVRAL
GITLMGHQKKILGSIQTMRAQLTSTQGPRRHL

| | | |
|---|---|---|
| EPH1g, 252417872 | SEQ ID NO: 13 | 1925 bp |
| DNA Sequence | ORF Start: at 2 | ORF Stop: end of sequence |

CACCAGATCTATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGGGCGGGAAGATCCCCATCCGCTGGACGGCCCCAGAGGCCATCGCCTTCCGCACC
TTCTCCTCGGCCAGCGACGTGTGGAGCTTCGGCGTGGTCATGTGGGAGGTGCTGGCCTATGGGGAGCG
GCCCTACTGGAACATGACCAACCGGGATGTCATCAGCTCTGTGGAGGAGGGGTACCGCCTGCCCGCAC
CCATGGGCTGCCCCCACGCCCTGCACCAGCTCATGCTCGACTGTTGGCACAAGGACCGGGCGCAGCGG
CCTCGCTTCTCCCAGATTGTCAGTGTCCTCGATGCGCTCATCCGCAGCCCTGAGAGTCTCAGGGCCAC
CGCCACAGTCAGCAGGTGCCCACCCCCTGCCTTCGTCCGGAGCTGCTTTGACCTCCGAGGGGGCAGCG
GTGGCGGTGGGGGCCTCACCGTGGGGGACTGGCTGGACTCCATCCGCATGGGCCGGTACCGAGACCAC
TTCGCTGCGGGCGGATACTCCTCTCTGGGCATGGTGCTACGCATGAACGCCCAGGACGTGCGCGCCCT
GGGCATCACCCTCATGGGCCACCAGAAGAAGATCCTGGGCAGCATTCAGACCATGCGGGCCCAGCTGA
CCAGCACCCAGGGGCCCCGCCGGCACCTCTGAAAGCTTGGC

| | | | |
|---|---|---|---|
| EPH1g, 252417872 | SEQ ID NO: 14 | 304 aa | MW at 33793.5kD |
| Protein Sequence | | | |

TRSIHIEKIIGSGDSGEVCYGRLRVPGQRDVPVAIKALKAGYTERQRRDFLSEASIMGQFDHPNIIRL
EGVVTRGRLAMIVTEYMENGSLDTFLRGGKIPIRWTAPEAIAFRTFSSASDVWSFGVVMWEVLAYGER
PYWNMTNRDVISSVEEGYRLPAPMGCPHALHQLMLDCWHKDRAQRPRFSQIVSVLDALIRSPESLRAT
ATVSRCPPPAFVRSCFDLRGGSGGGGGLTVGDWLDSIRMGRYRDHFAAGGYSSLGMVLRMNAQDVRAL
GITLMGHQKKILGSIQTMRAQLTSTQGPRRHL

| | | |
|---|---|---|
| EPH1h, 252417876 | SEQ ID NO: 15 | 925 bp |
| DNA Sequence | ORF Start: at 2 | ORF Stop: end of sequence |

CACCAGATCTATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGTCTCTCTGG
ACACCTTCCTGAGGGGCGGGAAGATCCCCATCCGCTGGACGGCCCCAGAGGCCATCGCCTTCCGCACC
TTCTCCTCGGCCAGCGACGTGTGGAGCTTCGGCGTGGTCATGTGGGGGGTGCTGGCCTATGGGGAGCG
GCCCTACTGGAACATGACCAACCGGGATGTCATCAGCTCTGTGGAGGAGGGGTACCGCCTGCCCGCAC
CCATGGGCTGCCCCCACGCCCTGCACCAGCTCATGCTCGACTGTTGGCACAAGGACCGGGCGCAGCGG
CCTCGCTTCTCCCAGATTGTCAGTGTCCTCGATGCGCTCATCCGCAGCCCTGAGAGTCTCAGGGCCAC
CGCCACAGTCAGCAGGTGCCCACCCCCTGCCTTCGTCCGGAGCTGCTTTGACCTCCGAGGGGGCAGCG
GTGGCGGTGGGGGCCTCACCGTGGGGGACTGGCTGGACTCCATCCGCATGGGCCGGTACCGAGACCAC
TTCGCTGCGGGCGGATACTCCTCTCTGGGCATGGTGCTACGCATGAACGCCCAGGACGTGCGCGCCCT
GGGCATCACCCTCATGGGCCACCAGAAGAAGATCCTGGGCAGCATTCAGACCATGCGGGCCCAGCTGA
CCAGCACCCAGGGGCCCCGCCGGCACCTCTGAAAGCTTGGC

| | | | |
|---|---|---|---|
| EPH1h, 252417876 | SEQ ID NO: 16 | 304 aa | MW at 33763.5kD |
| Protein Sequence | | | |

TRSIHIEKIIGSGDSGEVCYGRLRVPGQRDVPVAIKALKAGYTERQRRDFLSEASIMGQFDHPNIIRL
EGVVTRGRLAMIVTEYMENVSLDTFLRGGKIPIRWTAPEAIAFRTFSSASDVWSFGVVMWGVLAYGER
PYWNMTNRDVISSVEEGYRLPAPMGCPHALHQLMLDCWHKDRAQRPRFSQIVSVLDALIRSPESLRAT
ATVSRCPPPAFVRSCFDLRGGSGGGGGLTVGDWLDSIRMGRYRDHFAAGGYSSLGMVLRMNAQDVRAL
GITLMGHQKKILGSIQTMRAQLTSTQGPRRHL

| | | |
|---|---|---|
| EPH1i, 248213633 | SEQ ID NO: 17 | 1762 bp |
| DNA Sequence | ORF Start: at 2 | ORF Stop: end of sequence |

AGGCTCCGCGGCCGCCCCCTTCACCAGATCTGCAGCCCCGTCCCAGGTGGTGGTGATCCGTCAAGAGC
GGGCGGGGCAGACCAGCGTCTCGCTGCTGTGGCAGGAGCCCGAGCAGCCGAACGGCATCATCCTGGAG
TATGAGATCAAGTACTACGAGAAGGACAAGGAGATGCAGAGCTACTCCACCCTCAAGGCCGTCACCAC
CAGAGCCACCGTCTCCGGCCTCAAGCCGGGCACCCGCTACGTGTTCCAGGTCCGAGCCCGCACCTCAG
CAGGCTGTGGCCGCTTCAGCCAGGCCATGGAGGTGGAGACCGGGAAACCCCGGCCCCGCTATGACACC
AGGACCATTGTCTGGATCTGCCTGACGCTCATCACGGGCCTGGTGGTGCTTCTGCTCCTGCTCATCTG
CAAGAAGAGGCACTGTGGCTACAGCAAGGCCTTCCAGGACTCGGACGAGGAGAAGATGCACTATCAGA
ATGGACAGGCACCCCCACCTGTCTTCCTGCCTCTGCATCACCCCCCGGGAAAGCTCCCAGAGCCCCAG
TTCTATGCGGAACCCCACACCTACGAGGAGCCAGGCCGGGCGGGCCGCACTTTCACTCGGGAGATCGA
GGCCTCTAGGATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGACCCACGACGGGCAGTTCACCATCATGCAGCTGGTGGGCATGCTGAGAGGAGTG
GGTGCCGGCATGCGCTACCTCTCAGACCTGGGCTATGTCCACCGAGACCTGGCCGCCCGCAACGTCCT
GGTTGACAGCAACCTGGTCTGCAAGGTGTCTGACTTCGGGCTCTCACGGGTGCTGGAGGACGACCCGG
ATGCTGCCTACACCACCACGGGCGGGAAGATCCCCATCCGCTGGACGGCCCCAGAGGCCATCGCCTTC
CGCACCTTCTCCTCGGCCAGCGACGTGTGGAGCTTCGGCGTGGTCATGTGGGAGGTGCTGGCCTATGG
GGAGCGGCCCTACTGGAACATGACCAACCGGGATGTCATCAGCTCTGTGGAGGAGGGGTACCGCCTGC
CCGCACCCATGGGCTGCCCCCACGCCCTGCACCAGCTCATGCTCGACTGTTGGCACAAGGACCGGGCG
CAGCGGCCTCGCTTCTCCCAGATTGTCAGTGTCCTCGATGCGCTCATCCGCAGCCCTGAGAGTCTCAG
GGCCACCGCCACAGTCAGCAGGTGCCCACCCCCTGCCTTCGTCCGGAGCTGCTTTGACCTCCGAGGGG
GCAGCGGTGGCGGTGGGGGCCTCACCGTGGGGGACTGGCTGGACTCCATCCGCATGGGCCGGTACCGA
GACCACTTCGCTGCGGGCGGATACTCCTCTCTGGGCATGGTGCTACGCATGAACGCCCAGGACGTGCG
CGCCCTGGGCATCACCCTCATGGGCCACCAGAAGAAGATCCTGGGCAGCATTCAGACCATGCGGGCCC
AGCTGACCAGCACCCAGGGGCCCCGCCGGCACCTCTGAAAGCTTGGCAAGGGTGGGCGCGCC

| | | | |
|---|---|---|---|
| EPH1i, 248213633 | SEQ ID NO: 18 | 578 aa | MW at 64374.1kD |
| Protein Sequence | | | |

GSAAAPFTRSAAPSQVVVIRQERAGQTSVSLLWQEPEQPNGIILEYEIKYYEKDKEMQSYSTLKAVTT
RATVSGLKPGTRYVFQVRARTSAGCGRFSQAMEVETGKPRPRYDTRTIVWICLTLITGLVVLLLLLIC
KKRHCGYSKAFQDSDEEKMHYQNGQAPPPVFLPLHHPPGKLPEPQFYAEPHTYEEPGRAGRSFTREIE
ASRIHIEKIIGSGDSGEVCYGRLRVPGQRDVPVAIKALKAGYTERQRRDFLSEASIMGQFDHPNIIRL
EGVVTRGRLAMIVTEYMENGSLDTFLRTHDGQFTIMQLVGMLRGVGAGMRYLSDLGYVHRDLAARNVL
VDSNLVCKVSDFGLSRVLEDDPDAAYTTTGGKIPIRWTAPEAIAFRTFSSASDVWSFGVVMWEVLAYG
ERPYWNMTNRDVISSVEEGYRLPAPMGCPHALHQLMLDCWHKDRAQRPRFSQIVSVLDALIRSPESLR
ATATVSRCPPPAFVRSCFDLRGGSGGGGGLTVGDWLDSIRMGRYRDHFAAGGYSSLGMVLRMNAQDVR
ALGITLMGHQKKILGSIQTMRAQLTSTQGPRRHL

| | | |
|---|---|---|
| EPH1j, 248213637 | SEQ ID NO: 19 | 1762 bp |
| DNA Sequence | ORF Start: at 2 | ORF Stop: end of sequence |

AGGCTCCGCGGCCGCCCCCTTCACCAGATCTGCAGCCCCGTCCCAGGTGGTGGTGATCCGTCAAGAGC
GGGCGGGGCAGACCAGCGTCTCGCTGCTGTGGCAGGAGCCCGAGCAGCCGAACGGCATCATCCTGGAG
TATGAGATCAAGTACTACGAGAAGGACAAGGAGATGCAGAGCTACTCCACCCTCAAGGCCGTCACCAC
CAGAGCCACCGTCTCCGGCCTCAAGCCGGGCACCCGCTACGTGTTCCAGGTCCGAGCCCGCACCTCAG
CAGGCTGTGGCCGCTTCAGCCAGGCCATGGAGGTGGAGACCGGGAAACCCCGGCCCCGCTATGACACC
AGGACCATTGTCTGGATCTGCCTGACGCTCATCACGGGCCTGGTGGTGCTTCTGCTCCTGCTCATCTG
CAAGAAGAGGCACTGTGGCTACAGCAAGGCCTTCCAGGACTCGGACGAGGAGAAGATGCACTATCAGA
ATGGACAGGCACCCCCACCTGTCTTCCTGCCTCTGCATCACCCCCCGGGAAAGCTCCCAGAGCCCCAG
TTCTATGCGCAACCCCACACCTACGAGGAGCCAGGCCGGGCGGGCCGCAGTTTCACTCGGGAGATCGA
GGCCTCTAGGATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGACCCACGACGGGCAGTTCACCATCATGCAGCTGGTGGGCATGCTGAGAGGAGTG
GGTGCCGGCATGCGCTACCTCTCAGACCTGGGCTATGTCCACCGAGACCTGGCCGCCCGCAACGTCCT
GGTTGACAGCAACCTGGTCTGCAAGGTGTCTGACTTCGGGCTCTCACGGGTGCTGGAGGACGACCCGG
ATGCTGCCTACACCACCACGGGCGGGAAGATCCCCATCCGCTGGACGGCCCCAGAGGCCATCGCCTTC
CGCACCTTCTCCTCGGCCAGCGACGTGTGGAGCTTCGGCGTGGTCATGTGGGAGGTGCTGGCCTATGG
GGAGCGGCCCTACTGGAACATGACCAACCGGGATGTCATCAGCTCTGTGGAGGAGGGGTACCGCCTGC
CCGCACCCATGGGCTGCCCCCACGCCCTGCACCAGCTCATGCTCGACTGTTGGCACAAGGACCGGGCG
CAGCGGCCTCGCTTCTCCCAGATTGTCAGTGTCCTCGATGCGCTCATCCGCAGCCCTGAGAGTCTCAG
GGCCACCGCCACAGTCAGCAGGTGCCCACCCCCTGCCTTCGTCCGGAGCTGCTTTGACCTCCGAGGGG
GCAGCGGTGGCGGTGGGGGCCTCACCGTGGGGGACTGGCTGGACTCCATCCGCATGGGCCGGTACCGA
GACCACTTCGCTGCGGGCGGATACTCCTCTCTGGGCATGGTGCTACGCATGAACGCCCAGGACGTGCG
CGCCCTGGGCATCACCCTCATGGGCCACCAGAAGAAGATCCTGGGCAGCATTCAGACCATGCGGGCCC
AGCTGACCAGCACCCAGGGGCCCCGCCGGCACCTCTGAAAGCTTGGCAAGGGTGGGCGCGCC

| | | | |
|---|---|---|---|
| EPH1j, 248213637 | SEQ ID NO: 20 | 1578 aa | MW at 64373.1kD |
| Protein Sequence | | | |

GSAAAPFTRSAAPSQVVVIRQERAGQTSVSLLWQEPEQPNGIILEYEIKYYEKDKEMQSYSTLKAVTT
RATVSGLKPGTRYVFQVRARTSAGCGRFSQAMEVETGKPRPRYDTRTIVWICLTLITGLVVLLLLLIC
KKRHCGYSKAFQDSDEEKMHYQNGQAPPPVFLPLHHPPGKLPEPQFYAQPHTYEEPGRAGRSFTREIE
ASRIHIEKIIGSGDSGEVCYGRLRVPGQRDVPVAIKALKAGYTERQRRDFLSEASIMGQFDHPNIIRL
EGVVTRGRLAMIVTEYMENGSLDTFLRTHDGQFTIMQLVGMLRGVGAGMRYLSDLGYVHRDLAARNVL
VDSNLVCKVSDFGLSRVLEDDPDAAYTTTGGKIPIRWTAPEAIAFRTFSSASDVWSFGVVMWEVLAYG
ERPYWNMTNRDVISSVEEGYRLPAPMGCPHALHQLMLDCWHKDRAWRPRFSQIVSVLDALIRSPESLR
ATATVSRCPPPAFVRSCFDLRGGSGGGGGLTVGDWLDSIRMGRYRDHFAAGGYSSLGMVLRMNAQDVR
ALGITLMGHQKKILGSIQTMRAQLTSTQGPRRHL

| | | |
|---|---|---|
| EPH1k, 248209276 | SEQ ID NO: 21 | 1726 bp |
| DNA Sequence | ORF Start: at 2 | ORF Stop: end of sequence |

CACCAGATCTGCAGCCCCGTCCCAGGTGGTGGTGATCCGTCAAGAGCGGGCGGGGCAGACCAGCGTCT
CGCTGCTGTGGCAGGAGCCCGAGCAGCCGAACGGCATCATCCTGGAGTATGAGATCAAGTACTACGAG
AAGGACAAGGAGATGCAGAGCTACTCCACCCTCAAGGCCGTCACCACCAGAGCCACCGTCTCCGGCCT
CAAGCCGGGCACCCGCTACGTGTTCCAGGTCCGAGCCCGCACCTCAGCAGGCTGTGGCCGCTTCAGCC
AGGCCATGGAGGTGGAGACCGGGAAACCCCGGCCCCGCTATGACACCAGGACCATTGTCTGGATCTGC
CTGACGCTCATCACGGGCCTGGTGGTGCTTCTGCTCCTGCTCATCTGCAAGAAGAGGCACTGTGGCTA
CAGCAAGGCCTTCCAGGACTCGGACGAGGAGAAGATGCACTATCAGAATGGACAGGCACCCCCACCTG
TCTTCCTGCCTCTGCATCACCCCCCGGGAAAGCTCCCAGAGCCCCAGTTCTATGCGGAACCCCACACC
TACGAGGAGCCAGGCCGGGCGGGCCGCAGTTTCACTCGGGAGATCGAGGCCTCTAGGATCCACATCGA
GAAAATCATCGGCTCTGGAGACTCCGGGGAAGTCTGCTACGGGAGGCTGCGGGTGCCAGGGCAGCGGG
ATGTGCCCGTGGCCATCAAGGCCCTCAAAGCCGGCTACACGGAGAGACAGAGGCGGGACTTCCTGAGC
GAGGCGTCCATCATGGGGCAATTCGACCATCCCAACATCATCCGCCTCGAGGGTGTCGTCACCCGTGG
CCGCCTGGCAATGATTGTGACTGAGTACATGGAGAACGGCTCTCTGGACACCTTCCTGAGGACCCACG
ACGGGCAGTTCACCATCATGCAGCTGGTGGGCATGCTGAGAGGAGTGGGTGCCGGCATGCGCTACCTC
TCAGACCTGGGCTATGTCCACCGAGACCTGGCCGCCCGCAACGTCCTGGTTGACAGCAACCTGGTCTG
CAAGGTGTCTGACTTCGGGCTCTCACGGGTGCTGGAGGACGACCCGGATGCTGCCTACACCACCACGG
GCGGGAAGATCCCCATCCGCTGGACGGCCCCAGAGGCCATCGCCTTCCGCACCTTCTCCTCGGCCAGC
GACGTGTGGAGCTTCGGCGTGGTCATGTGGGAGGTGCTGGCCTATGGGGAGCGGCCCTACTGGAACAT
GACCAACCGGGATGTCATCAGCTCTGTGGAGGAGGGGTACCGCCTGCCCGCACCCATGGGCTGCCCCC
ACGCCCTGCACCAGCTCATGCTCGACTGTTGGCACAAGGACCGGGCGCAGCGGCCTCGCTTCTCCCAG
ATTGTCAGTGTCCTCGATGCGCTCATCCGCAGCCCTGAGAGTCTCAGGGCCACCGCCACAGTCAGCAG
GTGCCCACCCCCTGCCTTCGTCCGGAGCTGCTTTGACCTCCGAGGGGGCAGCGGTGGCGGTGGGGGCC
TCACCGTGGGGGACTGGCTGGACTCCATCCGCATGGGCCGGTACCGAGACCACTTCGCTGCGGGCGGA
TACTCCTCTCTGGGCATGGTGCTACGCATGAACGCCCAGGACGTGCGCGCCCTGGGCATCACCCTCAT
GGGCCACCAGAAGAAGATCCTGGGCAGCATTCAGACCATGCGGGCCCAGCTGACCAGCACCCAGGGGC
CCCGCCGGCACCTCTGAAAGCTTGGC

| | | | |
|---|---|---|---|
| EPH1k, 248209276 | SEQ ID NO: 22 | 571 aa | MW at 63772.4kD |
| Protein Sequence | | | |

TRSAAPSQVVVIRQERAGQTSVSLLWQEPEQPNGIILEYEIKYYEKDKEMQSYSTLKAVTTRATVSGL
KPGTRYVFQVRARTSAGCGRFSQAMEVETGKPRPRYDTRTIVWICLTLITGLVVLLLLLICKKRHCGY
SKAFQDSDEEKMHYQNGQAPPPVFLPLHHPPGKLPEPQFYAEPHTYEEPGRAGRSFTREIEASRIHIE
KIIGSGDSGEVCYGRLRVPGQRDVPVAIKALKAGYTERQRRDFLSEASIMGQFDHPNIIRLEGVVTRG
RLAMIVTEYMENGSLDTFLRTHDGQFTIMQLVGMLRGVGAGMRYLSDLGYVHRDLAARNVLVDSNLVC
KVSDFGLSRVLEDDPDAAYTTTGGKIPIRWTAPEAIAFRTFSSASDVWSFGVVMWEVLAYGERPYWNM
TNRDVISSVEEGYRLPAPMGCPHALHQLMLDCWHKDRAQRPRFSQIVSVLDALIRSPESLRATATVSR
CPPPAFVRSCFDLRGGSGGGGGLTVGDWLDSIRMGRYRDHFAAGGYSSLGMVLRMNAQDVRALGITLM
GHQKKILGSIQTMRAQLTSTQGPRRHL

| | | |
|---|---|---|
| EPH1l, 248213660 | SEQ ID NO: 23 | 1433 bp |
| DNA Sequence | ORF Start at 2 | ORF Stop: end of sequence |

AGGCTCCGCGGCCGCCCCCTTCACCAGATCTGCAGCCCCGTCCCAGGTGGTGGTGATCCGTCAAGAGC
GGGCGGGGCAGACCAGCGTCTCGCTGCTGTGGCAGGAGCCCGAGCAGCCGAACGGCATCATCCTGGAG
TATGAGATCAAGTACTACGAGAAGGACAAGGAGATGCAGAGCTACTCCACCCTCAAGGCCGTCACCAC
CAGAGCCACCGTCTCCGGCCTCAAGCCGGGCACCCGCTACGTGTTCCAGGTCCGAGCCCGCACCTCAG
CAGGCTGTGGCCGCTTCAGCCAGGCCATGGAGGTGGAGACCGGGAAACCCCGGCCCCGCTATGACACC
AGGACCATTGTCTGGATCTGCCTGACGCTCATCACGGGCCTGGTGGTGCTTCTGCTCCTGCTCATCTG
CAAGAAGAGGCACTGTGGCTACAGCAAGGCCTTCCAGGACTCGGACGAGGAGAAGATGCACTATCAGA
ATGGACAGGCACCCCCACCTGTCTTCCTGCCTCTGCATCACCCCCCGGGAAAGCTCCCAGAGCCCCAG
TTCTATGCGGAACCCCACACCTACGAGGAGCCAGGCCGGGCGGGCCGCAGTTTCACTCGGGAGATCGA
GGCCTCTAGGATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGACCCACGACGGGCAGTTCACCATCATGCAGCTGGTGGGCATGCTGAGAGGAGTG
GGTGCCGGCATGCGCTACCTCTCAGACCTGGGCTATGTCCACCGAGACCTGGCCGCCCGCAACGTCCT
GGTTGACAGCAACCTGGTCTGCAAGGTGTCTGACTTCGGGCTCTCACGGGTGCTGGAGGACGACCCGG
ATGCTGCCTACACCACCACGGGCGGGAAGATCCCCATCCGCTGGACGGCCCCAGAGGCCATCGCCTTC
CGCACCTTCTCCTCGGCCAGCGACGTGTGGAGCTTCGGCGTGGTCATGTGGGAGGTGCTGGCCTATGG
GGAGCGGCCCTACTGGAACATGACCAACCGGGATGTCATCAGCTCTGTGGAGGAGGGGTACCGCCTGC
CCGCACCCATGGGCTGCCCCCACGCCCTGCACCAGCTCATGCTCGACTGTTGGCACAAGGACCGGGCG
CAGCGGCCTCGCTTCTCCCAGATTGTCAAGCTTGGCAAGGGTGGGCGCGCCGACCCAGCTTTCTTGTA
CAAG

| | | | |
|---|---|---|---|
| EPH1l, 248213660 | SEQ ID NO: 24 | 477 aa | MW at 53552.8kD |
| Protein Sequence | | | |

GSAAAPFTRSAAPSQVVVIRQERAGQTSVSLLWQEPEQPNGIILEYEIKYYEKDKEMQSYSTLKAVTT
RATVSGLKPGTRYVFQVRARTSAGCGRFSQAMEVETGKPRPRYDTRTIVWICLTLITGLVVLLLLLIC
KKRHCGYSKAFQDSDEEKMHYQNGQAPPPVFLPLHHPPGKLPEPQFYAEPHTYEEPGRAGRSFTREIE
ASRIHIEKIIGSGDSGEVCYGRLRVPGQRDVPVAIKALKAGYTERQRRDFLSEASIMGQFDHPNIIRL
EGVVTRGRLAMIVTEYMENGSLDTFLRTHDGQFTIMQLVGMLRGVGAGMRYLSDLGYVHRDLAARNVL
VDSNLVCKVSDFGLSRVLEDDPDAAYTTTGGKIPIRWTAPEAIAFRTFSSASDVWSFGVVMWEVLAYG
ERPYWNMTNRDVISSVEEGYRLPAPMGCPHALHQLMLDCWHKDRAQRPRFSQIVKLGKGGRADPAFLY
K

| | | |
|---|---|---|
| EPH1m, 248213680 | SEQ ID NO: 25 | 1411 bp |
| DNA Sequence | ORF Start: at 2 | ORF Stop: end of sequence |

AGGCTCCGCGGCCGCCCCCTTCACCAGATCTGCAGCCCCGTCCCAGGTGGTGGTGATCCGTCAAGAGC
GGGCGGGGCAGACCAGCGTCTCGCTGCTGTGGCAGGAGCCCGAGCAGCCGAACGGCATCATCCTGGAG
TATGAGATCAAGTACTACGAGAAGGACAAGGAGATGCAGAGCTACTCCACCCTCAAGGCCGTCACCAC
CAGAGCCACCGTCTCCGGCCTCAAGCCGGGCACCCGCTACGTGTTCCAGGTCCGAGCCCGCACCTCAG
CAGGCTGTGGCCGCTTCAGCCAGGCCATGGAGGTGGAGACCGGGAAACCCCGGCCCCGCTATGACACC
AGGACCATTGTCTGGATCTGCCTGACGCTCATCACGGGCCTGGTGGTGCTTCTGCTCCTGCTCATCTG
CAAGAAGAGGCACTGTGGCTACAGCAAGGCCTTCCAGGACTCGGACGAGGAGAAGATGCACTATCAGA
ATGGACAGGCACCCCCACCTGTCTTCCTGCCTCTGCATCACCCCCCGGGAAAGCTCCCAGAGCCCCAG
TTCTATGCGCAACCCCACACCTACGAGGAGCCAGGCCGGGCGGGCCGCAGTTTCACTCGGGAGATCGA
GGCCTCTAGGATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGACCCACGACGGGCAGTTCACCATCATGCAGCTGGTGGGCATGCTGAGAGGAGTG
GGTGCCGGCATGCGCTACCTCTCAGACCTGGGCTATGTCCACCGAGACCTGGCCGCCCGCAACGTCCT
GGTTGACAGCAACCTGGTCTGCAAGGTGTCTGACTTCGGGCTCTCACGGGTGCTGGAGGACGACCCGG
ATGCTGCCTACACCACCACGGGCGGGAAGATCCCCATCCGCTGGACGGCCCCAGAGGCCATCGCCTTC
CGCACCTTCTCCTCGGCCAGCGACGTGTGGAGCTTCGGCGTGGTCATGTGGGAGGTGCTGGCCTATGG
GGAGCGGCCCTACTGGAACATGACCAACCGGGATGTCATCAGCTCTGTGGAGGAGGGGTACCGCCTGC
CCGCACCCATGGGCTGCCCCCACGCCCTGCACCAGCTCATGCTCGACTGTTGGCACAAGGACCGGGCG
CAGCGGCCTCGCTTCTCCCAGATTGTCAAGCTTGGCAAGGGTGGGCGCGCC

| | | | |
|---|---|---|---|
| EPH1m, 248213680 | SEQ ID NO: 26 | 470 aa | MW at 52716.9kD |
| Protein Sequence | | | |

GSAAAPFTRSAAPSQVVVIRQERAGQTSVSLLWQEPEQPNGIILEYEIKYYEKDKEMQSYSTLKAVTT
RATVSGLKPGTRYVFQVRARTSAGCGRFSQAMEVETGKPRPRYDTRTIVWICLTLITGLVVLLLLLIC
KKRHCGYSKAFQDSDEEKMHYQNGQAPPPVFLPLHHPPGKLPEPQFYAQPHTYEEPGRAGRSFTREIE
ASRIHIEKIIGSGDSGEVCYGRLRVPGQRDVPVAIKALKAGYTERQRRDFLSEASIMGQFDHPNIIRL
EGVVTRGRLAMIVTEYMENGSLDTFLRTHDGQFTIMQLVGMLRGVGAGMRYLSDLGYVHRDLAARNVL
VDSNLVCKVSDFGLSRVLEDDPDAAYTTTGGKIPIRWTAPEAIAFRTFSSASDVWSFGVVMWEVLAYG
ERPYWNMTNRDVISSVEEGYRLPAPMGCPHALHQLMLDCWHKDRAQRPRFSQIVKLGKGGRA

| | | |
|---|---|---|
| EPH1n, 248213688 | SEQ ID NO: 27 | 1439 bp |
| DNA Sequence | ORF Start: at 2 | ORF Stop: end of sequence |

AGGCTCCGCGGCCGCCCCCTTCACCAGATCTGCAGCCCCGTCCCAGGTGGTGGTGATCCGTCAAGAGC
GGGCGGGGCAGACCAGCGTCTCGCTGCTGTGGCAGGAGCCCGAGCAGCCGAACGGCATCATCCTGGAG
TATGAGATCAAGTACTACGAGAAGGACAAGGAGATGCAGAGCTACTCCACCCTCAAGGCCGTCACCAC
CAGAGCCACCGTCTCCGGCCTCAAGCCGGGCACCCGCTACGTGTTCCAGGTCCGAGCCCGCACCTCAG
CAGGCTGTGGCCGCTTCAGCCAGGCCATGGAGGTGGAGACCGGGAAACCCCGGCCCCGCTATGACACC
AGGACCATTGTCTGGATCTGCCTGACGCTCATCACGGGCCTGGTGGTGCTTCTGCTCCTGCTCATCTG
CAAGAAGAGGCACTGTGGCTACAGCAAGGCCTTCCAGGACTCGGACGAGGAGAAGATGCACTATCAGA
ATGGACAGGCACCCCCACCTGTCTTCCTGCCTCTGCATCACCCCCCGGGAAAGCTCCCAGAGCCCCAG
TTCTATGCGGAACCCCACACCTACGAGGAGCCAGGCCGGGCGGGCCGCAGTTTCACTCGGGAGATCGA
GGCCTCTAGGATCCACATCGAGAAAATCATCGGCTCTGGAGACTCCGGGGAAGTCTGCTACGGGAGGC
TGCGGGTGCCAGGGCAGCGGGATGTGCCCGTGGCCATCAAGGCCCTCAAAGCCGGCTACACGGAGAGA
CAGAGGCGGGACTTCCTGAGCGAGGCGTCCATCATGGGGCAATTCGACCATCCCAACATCATCCGCCT
CGAGGGTGTCGTCACCCGTGGCCGCCTGGCAATGATTGTGACTGAGTACATGGAGAACGGCTCTCTGG
ACACCTTCCTGAGGACCCACGACGGGCAGTTCACCATCATGCAGCTGGTGGGCATGCTGAGAGGAGTG
GGTGCCGTCATGCGCTACCTCTCAGACCTGGGCTATGTCCACCGAGACCTGGCCGCCCGCAACGTCCT
GGTTGACAGCAACCTGGTCTGCAAGGTGTCTGACTTCGGGCTCTCACGGGTGCTGGAGGACGACCCGG
ATGCTGCCTACACCACCACGGGCGGGAAGATCCCCATCCGCTGGACGGCCCCAGAGGCCATCGCCTTC
CGCACCTTCTCCTCGGCCAGCGACGTGTGGAGCTTCGGCGTGGTCATGTGGGAGGTGCTGGCCTATGG
GGAGCGGCCCTACTGGAACATGACCAACCGGGATGTCATCAGCTCTGTGGAGGAGGGGTACCGCCTGC
CCGCACCCATGGGCTGCCCCCACGCCCTGCACCAGCTCATGCTCGACTGTTGGCACAAGGACCGGGCG
CAGCGGCCTCGCTTCTCCCAGATTGTCAAGCTTGGCAAGGGTGGGCGCGCGACCCAGCTTCTTGTACA
AGTTGGATATA

| | | | |
|---|---|---|---|
| EPH1n, 248213688 | SEQ ID NO: 28 | 479 aa | MW at 53762.1kD |
| Protein Sequence | | | |

GSAAAPFTRSAAPSQVVVIRQERAGQTSVSLLWQEPEQPNGIILEYEIKYYEKDKEMQSYSTLKAVTT
RATVSGLKPGTRYVFQVRARTSAGCGRFSQAMEVETGKPRPRYDTRTIVWICLTLITGLVVLLLLLIC
KKRHCGYSKAFQDSDEEKMHYQNGQAPPPVFLPLHHPPGKLPEPQFYAEPHTYEEPGRAGRSFTREIE
ASRIHIEKIIGSGDSGEVCYGRLRVPGQRDVPVAIKALKAGYTERQRRDFLSEASIMGQFDHPNIIRL
EGVVTRGRLAMIVTEYMENGSLDTFLRTHDGQFTIMQLVGMLRGVGAVMRYLSDLGYVHRDLAARNVL
VDSNLVCKVSDFGLSRVLEDDPDAAYTTTGGKIPIRWTAPEAIAFRTFSSASDVWSFGVVMWEVLAYG
ERPYWNMTNRDVISSVEEGYRLPAPMGCPHALHQLMLDCWHKDRAQRPRFSQIVKLGKGGRATQLLVQ
VGY

| | | |
|---|---|---|
| EPH1o, 248209393 | SEQ ID NO: 29 | 1375 bp |
| DNA Sequence | ORF Start: at 2 | ORF Stop: end of sequence |

CACCAGATCTGCAGCCCCGTCCCAGGTGGTGGTGATCCGTCAAGAGCGGGCGGGGCAGACCAGCGTCT
CGCTGCTGTGGCAGGAGCCCGAGCAGCCGAACGGCATCATCCTGGAGTATGAGATCAAGTACTACGAG
AAGGACAAGGAGATGCAGAGCTACTCCACCCTCAAGGCCGTCACCACCAGAGCCACCGTCTCCGGCCT
CAAGCCGGGCACCCGCTACGTGTTCCAGGTCCGAGCCCGCACCTCAGCAGGCTGTGGCCGCTTCAGCC
AGGCCATGGAGGTGGAGACCGGGAAACCCCGGCCCCGCTATGACACCAGGACCATTGTCTGGATCTGC
CTGACGCTCATCACGGGCCTGGTGGTGCTTCTGCTCCTGCTCATCTGCAAGAAGAGGCACTGTGGCTA
CAGCAAGGCCTTCCAGGACTCGGACGAGGAGAAGATGCACTATCAGAATGGACAGGCACCCCCACCTG
TCTTCCTGCCTCTGCATCACCCCCCGGGAAAGCTCCCAGAGCCCCAGTTCTATGCGGAACCCCACACC
TACGAGGAGCCAGGCCGGGCGGGCCGCAGTTTCACTCGGGAGATCGAGGCCTCTAGGATCCACATCGA
GAAAATCATCGGCTCTGGAGACTCCGGGGAAGTCTGCTACGGGAGGCTGCGGGTGCCAGGGCAGCGGG
ATGTGCCCGTGGCCATCAAGGCCCTCAAAGCCGGCTACACGGAGAGACAGAGGCGGGACTTCCTGAGC
GAGGCGTCCATCATGGGGCAATTCGACCATCCCAACATCATCCGCCTCGAGGGTGTCGTCACCCGTGG
CCGCCTGGCAATGATTGTGACTGAGTACATGGAGAACGGCTCTCTGGACACCTTCCTGAGGACCCACG
ACGGGCAGTTCACCATCATGCAGCTGGTGGGCATGCTGAGAGGAGTGGGTGCCGGCATGCGCTACCTC
TCAGACCTGGGCTATGTCCACCGAGACCTGGCCGCCCGCAACGTCCTGGTTGACAGCAACCTGGTCTG
CAAGGTGTCTGACTTCGGGCTCTCACGGGTGCTGGAGGACGACCCGGATGCTGCCTACACCACCACGG
GCGGGAAGATCCCCATCCGCTGGACGGCCCCAGAGGCCATCGCCTTCCGCACCTTCTCCTCGGCCAGC
GACGTGTGGAGCTTCGGCGTGGTCATGTGGGAGGTGCTGGCCTATGGGGAGCGGCCCTACTGGAACAT
GACCAACCGGGATGTCATCAGCTCTGTGGAGGAGGGGTACCGCCTGCCCGCACCCATGGGCTGCCCCC
ACGCCCTGCACCAGCTCATGCTCGACTGTTGGCACAAGGACCGGGCGCAGCGGCCTCGCTTCTCCCAG
ATTGTCAAGCTTGGC

| | | | |
|---|---|---|---|
| EPH1o, 248209393 | SEQ ID NO: 30 | 458 aa | MW at 51646.6kD |
| Protein Sequence | | | |

TRSAAPSQVVVIRQERAGQTSVSLLWQEPEQPNGIILEYEIKYYEKDKEMQSYSTLKAVTTRATVSGL
KPGTRYVFQVRARTSAGCGRFSQAMEVETGKPRPRYDTRTIVWICLTLITGLVVLLLLLICKKRHCGY
SKAFQDSDEEKMHYQNGQAPPPVFLPLHHPPGKLPEPQFYAEPHTYEEPGRAGRSFTREIEASRIHIE
KIIGSGDSGEVCYGRLRVPGQRDVPVAIKALKAGYTERQRRDFLSEASIMGQFDHPNIIRLEGVVTRG
RLAMIVTEYMENGSLDTFLRTHDGQFTIMQLVGMLRGVGAGMRYLSDLGYVHRDLAARNVLVDSNLVC
KVSDFGLSRVLEDDPDAAYTTTGGKIPIRWTAPEAIAFRTFSSASDVWSFGVVMWEVLAYGERPYWNM
TNRDVISSVEEGYRLPAPMGCPHALHQLMLDCWHKDRAQRPRFSQIVKLG

| | | |
|---|---|---|
| EPH1p, CG54020-03 | SEQ ID NO: 31 | 1545 bp |
| DNA Sequence | ORF Start: at 1 | ORF Stop: end of sequence |

GCGCGCGGCGAAGTGAATTTGCTGGACACGTCGACCATCCACGGGGACTGGGGCTGGCTCACGTATCC
GGCTCATGGGTGGGACTCCATCAACGAGGTGGACGAGTCCTTCCAGCCCATCCACACGTACCAGGTTT
GCAACGTCATGAGCCCCAACCAGAACAACTGGCTGCGCACGAGCTGGGTCCCCCGAGACGGCGCCCGG
CGCGTCTATGCTGAGATCAAGTTTACCCTGCGCGACTGCAACAGCATGCCTGGTGTGCTGGGCACCTG
CAAGGAGACCTTCAACCTCTACTACCTGGAGTCGGACCGCGACCTGGGGGCCAGCACACAAGAAAGCC
AGTTCCTCAAAATCGACACCATTGCGGCCGACGAGAGCTTCACAGGTGCCGACCTTGGTGTGCGGCGT
CTCAAGCTCAACACGGAGGTGCGCAGTGTGGGTCCCCTCAGCAAGCGCGGCTTCTACCTGGCCTTCCA
GGACATAGGTGCCTGCCTGGCCATCCTCTCTCTCCGCATCTACTATAAGAAGTGCCCTGCCATGGTGC
GCAATCTGGCTGCCTTCTCGGAGGCAGTGACGGGGGCCGACTCGTCCTCACTGGTGGAGGTGAGGGGC
CAGTGCGTGCGGCACTCAGAGGAGCGGGACACACCCAAGATGTACTGCAGCGCGGAGGGCGAGTGGCT
CGTGCCCATCGGCAAATGCGTGTGCAGTGCCGGCTACGAGGAGCGGCGGGATGCCTGTGTGGCCTGTG
AGCTGGGCTTCTACAAGTCAGCCCCTGGGGACCAGCTGTGTGCCCGCTGCCCTCCCCACAGCCACTCC
GCAGCTCCAGCCGCCCAAGCCTGCCACTGTGACCTCAGCTACTACCGTGCAGCCCTGGACCCGCCGTC
CTCAGCCTGCACCCGGCCACCCTCGGCACCAGTGAACCTGATCTCCAGTGTGAATGGGACATCAGTGA
CTCTGGAGTGGGCCCCTCCCCTGGACCCAGGTGGCCGCAGTGACATCACCTACAATGCCGTGTGCCGC
CGCTGCCCCTGGGCACTGAGCCGCTGCGAGGCATGTGGGAGCGGCACCCGCTTTGTGCCCCAGCAGAC
AAGCCTGGTGCAGGCCAGCCTGCTGGTGGCCAACCTGCTGGCCCACATGAACTACTCCTTCTGGATCG
AGGCCGTCAATGGCGTGTCCGACCTGAGCCCCGAGCCCCGCCGGGCCGCTGTGGTCAACATCACCACG
AACCAGGCAGCCCCGTCCCAGGTGGTGGTGATCCGTCAAGAGCGGGCGGGGCAGACCAGCGTCTCGCT
GCTGTGGCAGGAGCCCGAGCAGCCGAACGGCATCATCCTGGAGTATGAGATCAAGTACTACGAGAAGG
ACAAGGAGATGCAGAGCTACTCCACCCTCAAGGCCGTCACCACCAGAGCCACCGTCTCCGGCCTCAAG
CCGGGCACCCGCTACGTGTTCCAGGTCCGAGCCCGCACCTCAGCAGGCTGTGGCCGCTTCAGCCAGGC
CATGGAGGTGGAGACCGGGAAACCCCGGCCCCGCTATGACACCAGGACC

| | | | |
|---|---|---|---|
| EPH1p, CG54020-03 | SEQ ID NO: 32 | 515 aa | MW at 56842.5kD |
| Protein Sequence | | | |

ARGEVNLLDTSTIHGDWGWLTYPAHGWDSINEVDESFQPIHTYQVCNVMSPNQNNWLRTSWVPRDGAR
RVYAEIKFTLRDCNSMPGVLGTCKETFNLYYLESDRDLGASTQESQFLKIDTIAADESFTGADLGVRR
LKLNTEVRSVGPLSKRGFYLAFQDIGACLAILSLRIYYKKCPAMVRNLAAFSEAVTGADSSSLVEVRG
QCVRHSEERDTPKMYCSAEGEWLVPIGKCVCSAGYEERRDACVACELGFYKSAPGDQLCARCPPHSHS
AAPAAQACHCDLSYYRAALDPPSSACTRPPSAPVNLISSVNGTSVTLEWAPPLDPGGRSDITYNAVCR
RCPWALSRCEACGSGTRFVPQQTSLVQASLLVANLLAHMNYSFWIEAVNGVSDLSPEPRRAAVVNITT
NQAAPSQVVVIRQERAGQTSVSLLWQEPEQPNGIILEYEIKYYEKDKEMQSYSTLKAVTTRATVSGLK
PGTRYVFQVRARTSAGCGRFSQAMEVETGKPRPRYDTRT

| | | |
|---|---|---|
| EPH1q, CG54020-04 | SEQ ID NO: 33 | 2884 bp |
| DNA Sequence | ORF Start: ATG at 1 | ORF Stop: end of sequence |

ATGGCCCCCGCCCGGGGCCGCCTGCCCCCTGCGCTCTGGGTCGTCACGGCCGCGGCGGCGGCGGCCAC
CTGCGTGTCCGCGGCGCGCGGCGAAGTGAATTTGCTGGACACGTCGACCATCCACGGGGACTGGGGCT
GGCTCACGTATCCGGCTCATGGGTGGGACTCCATCAACGAGGTGGACGAGTCCTTCCAGCCCATCCAC
ACGTACCAGGTTTGCAATGTCATGAGCCCCAACCAGAACAACTGGCTGCGCACGAGCTGGGTCCCCCG
AGACGGCGCCCGGCGCGTCTATGCTGAGATCAAGTTTACCCTGCGCGACTGCAACAGCATGCCTGGTG
TGCTGGGCACCTGCAAGGAGACCTTCAACCTCTACTACCTGGAGTCGGACCGCGACCTGGGGGCCAGC
ACACAAGAAAGCCAGTTCCTCAAAATCGACACCATTGCGGCCGACGAGAGCTTCACAGGTGCCGACCT
TGGTGTGCGGCGTCTCAAGCTCAACACGGAGGTGCGCAGTGTGGGTCCCCTCAGCAAGCGCGGCTTCT
ACCTGGCCTTCCAGGACATAGGTGCCTGCCTGGCCATCCTCTCTCTCCGCATCTACTATAAGAAGTGC
CCTGCCATGGTGCGCAATCTGGCTGCCTTCTCGGAGGCAGTGACGGGGGCCGACTCGTCCTCACTGGT
GGAGGTGAGGGGCCAGTGCGTGCGGCACTCAGAGGAGCGGGACACACCCAAGATGTACTGCAGCGCGG
AGGGCGAGTGGCTCGTGCCCATCGGCAAATGCGTGTGCAGTGCCGGCTACGAGGAGCGGCGGGATGCC
TGTGTGGCCTGTGAGCTGGGCTTCTACAAGTCAGCCCCTGGGGACCAGCTGTGTGCCCGCTGCCCTCC
CCACAGCCACTCCGCAGCTCCAGCCGCCCAAGCCTGCCACTGTGACCTCAGCTACTACCGTGCAGCCC
TGGACCCGCCGTCCTCAGCCTGCACCCGGCCACCCTCGGCACCAGTGAACCTGATCTCCAGTGTGAAT
GGGACATCAGTGACTCTGGAGTGGGCCCCTCCCCTGGACCCAGGTGGCCGCAGTGACATCACCTACAA
TGCCGTGTGCCGCCGCTGCCCCTGGGCACTGAGCCGCTGCGAGGCATGTGGGAGCGGCACCCGCTTTG
TGCCCCAGCAGACAAGCCTGGTGCAGGCCAGCCTGCTGGTGGCCAACCTGCTGGCCCACATGAACTAC
TCCTTCTGGATCGAGGCCGTCAATGGCGTGTCCGACCTGAGCCCCGAGCCCCGCCGGGCCGCTGTAGT
CAACATCACCACGAACCAGGCAGCCCCGTCCCAGGTGGTGGTGATCCGTCAAGAGCGGGCGGGGCAGA
CCAGCGTCTCGCTGCTGTGGCAGGAGCCCGAGCAGCCGAACGGCATCATCCTGGAGTATGAGATCAAG
TACTACGAGAAGGACAAGGAGATGCAGAGCTACTCCACCCTCAAGGCCGTCACCACCAGAGCCACCGT
CTCCGGCCTCAAGCCGGGCACCCGCTACGTGTTCCAGGTCCGAGCCCGCACCCCAGCAGGCTGTGGCC
GCTTCAGCCAGGCCATGGAGGTGGAGACCGGGAAACCCCGGCCCCGCTATGACACCAGGACCATTGTC
TGGATCTGCCTGACGCTCATCACGGGCCTGGTGGTGCTTCTGCTCCTGCTCATCTGCAAGAAGAGGCA
CTGTGGCTACAGCAAGGCCTTCCAGGACTCGGACGAGGAGAAGATGCACTATCAGAATGGACAGGCAC
CCCCACCTGTCTTCCTGCCTCTGCATCACCCCCCGGGAAAGCTCCCAGAGCCCCAGTTCTATGCGGAA
CCCCACACCTACGAGGAGCCAGGCCGGGCGGGCCGCAGTTTCACTCGGGAGATCGAGGCCTCTAGGAT
CCACATCGAGAAAATCATCGGCTCTGGAGACTCCGGGGAAGTCTGCTACGGGAGGCTGCGGGTGCCAG
GGCAGCGGGATGTGCCCGTGGCCATCAAGGCCCTCAAAGCCGGCTACACGGAGAGACAGAGGCGGGAC
TTCCTGAGCGAGGCGTCCATCATGGGGCAATTCGACCATCCCAACATCATCCGCCTCGAGGGTGTCGT
CACCCGTGGCCGCCTGGCAATGATTGTGACTGAGTACATGGAGAACGGCTCTCTGGACACCTTCCTGA
GGGGCGGGAAGATCCCCATCCGCTGGACGGCCCCAGAGGCCATCGCCTTCCGCACCTTCTCCTCGGCC
AGCGACGTGTGGAGCTTCGGCGTGGTCATGTGGGAGGTGCTGGCCTATGGGGAGCGGCCCTACTGGAA
CATGACCAACCGGGATGTCATCAGCTCTGTGGAGGAGGGGTACCGCCTGCCCGCACCCATGGGCTGCC
CCCACGCCCTGCACCAGCTCATGCTCGACTGTTGGCACAAGGACCGGGCGCAGCGGCCTCGCTTCTCC
CAGATTGTCAGTGTCCTCGATGCGCTCATCCGCAGCCCTGAGAGTCTCAGGGCCACCGCCACAGTCAG
CAGGTGCCCACCCCCTGCCTTCGTCCGGAGCTGCTTTGACCTCCGAGGGGGCAGCGGTGGCGGTGGGG
GCCTCACCGTGGGGGACTGGCTGGACTCCATCCGCATGGGCCGGTACCGAGACCACTTCGCTGCGGGC
GGATACTCCTCTCTGGGCATGGTGCTACGCATGAACGCCCAGGACGTGCGCGCCCTGGGCATCACCCT
CATGGGCCACCAGAAGAAGATCCTGGGCAGCATTCAGACCATGCGGGCCCAGCTGACCAGCACCCAGG
GGCCCCGCCGGCACCTCTGATGTACAGCCAGCAGGGCCCAGGCAGCCACCGAGCCCACCCCAGGTCAT
GCCAGCGGCAGAGGACGTGAGGGGCTGG

| | | | |
|---|---|---|---|
| EPH1q, CG54020-04 | SEQ ID NO: 34 | 935 aa | MW at 103382.8kD |
| Protein Sequence | | | |

MAPARGRLPPALWVVTAAAAAATCVSAARGEVNLLDTSTIHGDWGWLTYPAHGWDSINEVDESFQPIH
TYQVCNVMSPNQNNWLRTSWVPRDGARRVYAEIKFTLRDCNSMPGVLGTCKETFNLYYLESDRDLGAS
TQESQFLKIDTIAADESFTGADLGVRRLKLNTEVRSVGPLSKRGFYLAFQDIGACLAILSLRIYYKKC
PAMVRNLAAFSEAVTGADSSSLVEVRGQCVRHSEERDTPKMYCSAEGEWLVPIGKCVCSAGYEERRDA
CVACELGFYKSAPGDQLCARCPPHSHSAAPAAQACHCDLSYYRAALDPPSSACTRPPSAPVNLISSVN
GTSVTLEWAPPLDPGGRSDITYNAVCRRCPWALSRCEACGSGTRFVPQQTSLVQASLLVANLLAHMNY
SFWIEAVNGVSDLSPEPRRAAVVNITTNQAAPSQVVVIRQERAGQTSVSLLWQEPEQPNGIILEYEIK
YYEKDKEMQSYSTLKAVTTRATVSGLKPGTRYVFQVRARTPAGCGRFSQAMEVETGKPRPRYDTRTIV
WICLTLITGLVVLLLLLICKKRHCGYSKAFQDSDEEKMHYQNGQAPPPVFLPLHHPPGKLPEPQFYAE
PHTYEEPGRAGRSFTREIEASRIHIEKIIGSGDSGEVCYGRLRVPGQRDVPVAIKALKAGYTERQRRD
FLSEASIMGQFDHPNIIRLEGVVTRGRLAMIVTEYMENGSLDTFLRGGKIPIRWTAPEAIAFRTFSSA
SDVWSFGVVMWEVLAYGERPYWNMTNRDVISSVEEGYRLPAPMGCPHALHQLMLDCWHKDRAQRPRFS
QIVSVLDALIRSPESLRATATVSRCPPPAFVRSCFDLRGGSGGGGGLTVGDWLDSIRMGRYRDHFAAG
GYSSLGMVLRMNAQDVRALGITLMGHQKKILGSIQTMRAQLTSTQGPRRHL

| | | |
|---|---|---|
| EPH1r, CG54020-05 | SEQ ID NO: 35 | 2884 bp |
| DNA Sequence | ORF Start: ATG at 1 | ORF Stop: end of sequence |

ATGGCCCCCGCCCGGGGCCGCCTGCCCCCTGCGCTCTGGGTCGTCACGGCCGCGGCGGCGGCGGCCAC
CTGCGTGTCCGCGGCGCGCGGCGAAGTGAATTTGCTGGACACGTCGACCATCCACGGGGACTGGGGCT
GGCTCACGTATCCGGCTCATGGGTGGGACTCCATCAACGAGGTGGACGAGTCCTTCCAGCCCATCCAC
ACGTACCAGGTTTGCAATGTCATGAGCCCCAACCAGAACAACTGGCTGCGCACGAGCTGGGTCCCCCG
AGACGGCGCCCGGCGCGTCTATGCTGAGATCAAGTTTACCCTGCGCGACTGCAACAGCATGCCTGGTG
TGCTGGGCACCTGCAAGGAGACCTTCAACCTCTACTACCTGGAGTCGGACCGCGACCTGGGGGCCAGC
ACACAAGAAAGCCAGTTCCTCAAAATCGACACCATTGCGGCCGACGAGAGCTTCACAGGTGCCGACCT
TGGTGTGCGGCGTCTCAAGCTCAACACGGAGGTGCGCAGTGTGGGTCCCCTCAGCAAGCGCGGCTTCT
ACCTGGCCTTCCAGGACATAGGTGCCTGCCTGGCCATCCTCTCTCTCCGCATCTACTATAAGAAGTGC
CCTGCCATGGTGCGCAATCTGGCTGCCTTCTCGGAGGCAGTGACGGGGGCCGACTCGTCCTCACTGGT
GGAGGTGAGGGGCCAGTGCGTGCGGCACTCAGAGGAGCGGGACACACCCAAGATGTACTGCAGCGCGG
AGGGCGAGTGGCTCGTGCCCATCGGCAAATGCGTGTGCAGTGCCGGCTACGAGGAGCGGCGGGATGCC
TGTGTGGCCTGTGAGCTGGGCTTCTACAAGTCAGCCCCTGGGGACCAGCTGTGTGCCCGCTGCCCTCC
CCACAGCCACTCCGCAGCTCCAGCCGCCCAAGCCTGCCACTGTGACCTCAGCTACTACCGTGCAGCCC
TGGACCCGCCGTCCTCAGCCTGCACCCGGCCACCCTCGGCACCAGTGAACCTGATCTCCAGTGTGAAT
GGGACATCAGTGACTCTGGAGTGGGCCCCTCCCCTGGACCCAGGTGGCCGCAGTGACATCACCTACAA
TGCCGTGTGCCGCCGCTGCCCCTGGGCACTGAGCCGCTGCGAGGCATGTGGGAGCGGCACCCGCTTTG
TGCCCCAGCAGACAAGCCTGGTGCAGGCCAGCCTGCTGGTGGCCAACCTGCTGGCCCACATGAACTAC
TCCTTCTGGATCGAGGCCGTCAATGGCGTGTCCGACCTGAGCCCCGAGCCCCGCCGGGCCGCTGTAGT
CAACATCACCACGAACCAGGCAGCCCCGTCCCAGGTGGTGGTGATCCGTCAAGAGCGGGCGGGGCAGA
CCAGCGTCTCGCTGCTGTGGCAGGAGCCCGAGCAGCCGAACGGCATCATCCTGGAGTATGAGATCAAG
TACTACGAGAAGGACAAGGAGATGCAGAGCTACTCCACCCTCAAGGCCGTCACCACCAGAGCCACCGT
CTCCGGCCTCAAGCCGGGCACCCGCTACGTGTTCCAGGTCCGAGCCCGCACCTCAGCAGGCTGTGGCC
GCTTCAGCCAGGCCATGGAGGTGGAGACCGGGAAACCCCGGCCCCGCTATGACACCAGGACCATTGTC
TGGATCTGCCTGACGCTCATCACGGGCCTGGTGGTGCTTCTGCTCCTGCTCATCTGCAAGAAGAGGCA
CTGTGGCTACAGCAAGGCCTTCCAGGACTCGGACGAGGAGAAGATGCACTATCAGAATGGACAGGCAC
CCCCACCTGTCTTCCTGCCTCTGCATCACCCCCCGGGAAAGCTCCCAGAGCCCCAGTTCTATGCGGAA
CCCCACACCTACGAGGAGCCAGGCCGGGCGGGCCGCAGTTTCACTCGGGAGATCGAGGCCTCTAGGAT
CCACATCGAGAAAATCATCGGCTCTGGAGACTCCGGGGAAGTCTGCTACGGGAGGCTGCGGGTGCCAG
GGCAGCGGGATGTGCCCGTGGCCATCAAGGCCCTCAAAGCCGGCTACACGGAGAGACAGAGGCGGGAC
TTCCTGAGCGAGGCGTCCATCATGGGGCAATTCGACCATCCCAACATCATCCGCCTCGAGGGTGTCGT
CACCCGTGGCCGCCTGGCAATGATTGTGACTGAGTACATGGAGAACGGCTCTCTGGACACCTTCCTGA
GGGGCGGGAAGATCCCCATCCGCTGGACGGCCCCAGAGGCCATCGCCTTCCGCACCTTCTCCTCGGCC
AGCGACGTGTGGAGCTTCGGCGTGGTCATGTGGGAGGTGCTGGCCTATGGGGAGCGGCCCTACTGGAA
CATGACCAACCGGGATGTCATCAGCTCTGTGGAGGAGGGGTACCGCCTGCCCGCACCCATGGGCTGCC
CCCACGCCCTGCACCAGCTCATGCTCGACTGTTGGCACAAGGACCGGGCGCAGCGGCCTCGCTTCTCC
CAGATTGTCAGTGTCCTCGATGCGCTCATCCGCAGCCCTGAGAGTCTCAGGGCCACCGCCACAGTCAG
CAGGTGCCCACCCCCTGCCTTCGTCCGGAGCTGCTTTGACCTCCGAGGGGGCAGCGGTGGCGGTGGGG
GCCTCACCGTGGGGGACTGGCTGGACTCCATCCGCATGGGCCGGTACCGAGACCACTTCGCTGCGGGC
GGATACTCCTCTCTGGGCATGGTGCTACGCATGAACGCCCAGGACGTGCGCGCCCTGGGCATCACCCT
CATGGGCCACCAGAAGAAGATCCTGGGCAGCATTCAGACCATGCGGGCCCAGCTGACCAGCACCCAGG
GGCCCCGCCGGCACCTCTGATGTACAGCCAGCAGGGCCCAGGCAGCCACCGAGCCCACCCCAGGTCAT
GCCAGCGGCAGAGGACGTGAGGGGCTGG

| | | | |
|---|---|---|---|
| EPH1r, CG54020-05 | SEQ ID NO: 36 | 935 aa | MW at 103372.7kD |
| Protein Sequence | | | |

MAPARGRLPPALWVVTAAAAAATCVSAARGEVNLLDTSTIHGDWGWLTYPAHGWDSINEVDESFQPIH
TYQVCNVMSPNQNNWLRTSWVPRDGARRVYAEIKFTLRDCNSMPGVLGTCKETFNLYYLESDRDLGAS
TQESQFLKIDTIAADESFTGADLGVRRLKLNTEVRSVGPLSKRGFYLAFQDIGACLAILSLRIYYKKC
PAMVRNLAAFSEAVTGADSSSLVEVRGQCVRHSEERDTPKMYCSAEGEWLVPIGKCVCSAGYEERRDA
CVACELGFYKSAPGDQLCARCPPHSHSAAPAAQACHCDLSYYRAALDPPSSACTRPPSAPVNLISSVN
GTSVTLEWAPPLDPGGRSDITYNAVCRRCPWALSRCEACGSGTRFVPQQTSLVQASLLVANLLAHMNY
SFWIEAVNGVSDLSPEPRRAAVVNITTNQAAPSQVVVIRQERAGQTSVSLLWQEPEQPNGIILEYEIK
YYEKDKEMQSYSTLKAVTTRATVSGLKPGTRYVFQVRARTSAGCGRFSQAMEVETGKPRPRYDTRTIV
WICLTLITGLVVLLLLLICKKRHCGYSKAFQDSDEEKMHYQNGQAPPPVFLPLHHPPGKLPEPQFYAE
PHTYEEPGRAGRSFTREIEASRIHIEKIIGSGDSGEVCYGRLRVPGQRDVPVAIKALKAGYTERQRRD
FLSEASIMGQFDHPNIIRLEGVVTRGRLAMIVTEYMENGSLDTFLRGGKIPIRWTAPEAIAFRTFSSA
SDVWSFGVVMWEVLAYGERPYWNMTNRDVISSVEEGYRLPAPMGCPHALHQLMLDCWHKDRAQRPRFS
QIVSVLDALIRSPESLRATATVSRCPPPAFVRSCFDLRGGSGGGGGLTVGDWLDSIRMGRYRDHFAAG
GYSSLGMVLRMNAQDVRALGITLMGHQKKILGSIQTMRAQLTSTQGPRRHL

CG54020-01 Splice Variants: Variants of the human Ephrin A8 receptor gene were obtained through direct cloning and/or comparison with public databases. A ClustalW comparison of the amino acid sequences of CG54020-01 and its variants is shown in Table 1B. CG54020-04 (SEQ ID: 34) and CG54020-05 (SEQ ID: 36) represent splice variants of the Ephrin A8 receptor that lack exon 12.

A ClustalW comparison of the above protein sequences yields the following sequence alignment shown in Table 1B.

TABLE 1B
Comparison of the EPH1 protein seqnences.

| | |
|---|---|
| EPH1a | MAPARGRLPPALWVVTAAAAAATCVSAARGEVNLLDTSTIHGDWGWLTYPAHGWDSINEV |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | ------------------------------------------------------------ |
| EPH1r | ------------------------------------------------------------ |
| EPH1a | DESFQPIHTYQVCNVMSPNQNNWLRTSWVPRDGARRVYAEIKFTLRDCNSMPGVLGTCKE |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | ------------------------------------------------------------ |
| EPH1r | ------------------------------------------------------------ |
| EPH1a | TFNLYYLESDRDLGASTQESQFLKIDTIAADESFTGADLGVRRLKLNTEVRSVGPLSKRG |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | ------------------------------------------------------------ |
| EPH1r | ------------------------------------------------------------ |
| EPH1a | FYLAFQDIGACLAILSLRIYYKKCPAMVRNLAAFSEAVTGADSSSLVEVRGQCVRHSEER |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPh1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | ------------------------------------------------------------ |
| EPH1r | ------------------------------------------------------------ |
| EPH1a | DTPKMYCSAEGEWLVPIGKCVCSAGYEERRDACVACELGFYKSAPGDQLCARCPPHSHSA |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | ------------------------------------------------------------ |
| EPH1r | ------------------------------------------------------------ |
| EPH1a | APAAQACHCDLSYYRAALDPPSSACTRPPSAPVNLISSVNGTSVTLEWAPPLDPGGRSDI |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPE1o | ------------------------------------------------------------ |
| EPH1p | ------------------------------------------------------------ |
| EPH1g | ------------------------------------------------------------ |
| EPH1r | ------------------------------------------------------------ |
| EPH1a | TYNAVCRRCPWALSRCEACGSGTRFVPQQTSLVQASLLVANLLAHMNYSFWIEAVNGVSD |
| EPH1b | ----------------------------------------------------ARGEVNLL |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ----------------------------------------------------ARGEVNLL |
| EPH1q | -------------------------MAPARGRLPPALWVVTAAAAAATCVSAARGEVNLL |
| EPH1r | -------------------------MAPARGRLPPALWVVTAAAAAATCVSAARGEVNLL |
| EPH1a | LSPEPRRAAVVNITTNQAAPSQVVVIRQERAGQTSVSLLWQEPEQPNGIILEYEIKYYEK |
| EPH1b | DTSTIHGDWGWLTYPAHGWDSINEVDESFQPIHTYQVCNVMSPNQNNWLRTSWVPRDGAR |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | -------GSAAAPFTRSAAPSQVVVIRQERAGQTSVSLLWQEPEQPNGIILEYEIKYYEK |
| EPH1j | -------GSAAAPFTRSAAPSQVVVIRQERAGQTSVSLLWQEPEQPNGIILEYEIKYYEK |
| EPH1k | --------------TRSAAPSQVVVIRQERAGQTSVSLLWQEPEQPNGIILEYEIKYYEK |
| EPH1l | -------GSAAAPFTRSAAPSQVVVIRQERAGQTSVSLLWQEPEQPNGIILEYEIKYYEK |
| EPH1m | -------GSAAAPFTRSAAPSQVVVIRQERAGQTSVSLLWQEPEQPNGIILEYEIKYYEK |
| EPH1n | -------GSAAAPFTRSAAPSQVVVIRQERAGQTSVSLLWQEPEQPNGIILEYEIKYYEK |
| EPH1o | --------------TRSAAPSQVVVIRQERAGQTSVSLLWQEPEQPNGIILEYEIKYYEK |
| EPH1p | DTSTIHGDWGWLTYPAHGWDSINEVDESFQPIHTYQVCNVMSPNQNNWLRTSWVPRDGAR |
| EPH1q | DTSTIHGDWGWLTYPAHGWDSINEVDESFQPIHTYQVCNVMSPNQNNWLRTSWVPRDGAR |
| EPH1r | DTSTIHGDWGWLTYPAHGWDSINEVDESFQPIHTYQVCNVMSPNQNNWLRTSWVPRDGAR |
| EPH1a | DKEMQSYSTLKAVTTRATVSGLKPGTRYVFQVRARTSAGCGR-FSQAMEVETGKPRPRYD |
| EPH1b | RVYAEIKFTLRDCNSMPGVLGTCKETFNLYYLESDRDLGASTQESQFLKIDTIAADESFT |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | DKEMQSYSTLKAVTTRATVSGLKPGTRYVFQVRARTSAGCGR-FSQAMEVETGKPRPRYD |
| EPH1j | DKEMQSYSTLKAVTTRATVSGLKPGTRYVFQVRARTSAGCGR-FSQAMEVETGKPRPRYD |
| EPH1k | DKEMQSYSTLKAVTTRATVSGLKPGTRYVFQVRARTSAGCGR-FSQAMEVETGKPRPRYD |
| EPH1l | DKEMQSYSTLKAVTTRATVSGLKPGTRYVFQVRARTSAGCGR-FSQAMEVETGKPRPRYD |
| EPH1m | DKEMQSYSTLKAVTTRATVSGLKPGTRYVFQVRARTSAGCGR-FSQAMEVETGKPRPRYD |
| EPH1n | DKEMQSYSTLKAVTTRATVSGLKPGTRYVFQVRARTSACCGR-FSQAMEVETGKPRPRYD |
| EPH1o | DKEMQSYSTLKAVTTRATVSGLKPGTRYVFQVRARTSAGCGR-FSQAMEVETGKPRPRYD |
| EPH1p | RVYAEIKFTLRDCNSMPGVLGTCKETFNLYYLESDRDLGASTQESQFLKIDTIAADESFT |
| EPH1q | RVYAEIKFTLRDCNSMPGVLGTCKETFNLYYLESDRDLGASTQESQFLKIDTIAADESFT |
| EPH1r | RVYAEIKFTLRDCNSMPGVLGTCKETFNLYYLESDRDLGASTQESQFLKIDTIAADESFT |
| EPH1a | ---------------------TRTIVWICLTLITGLVVLLLLLICKKRHCGYSKAFQDSD |
| EPH1b | GADLGVRRLKLNTEVRSVGPLSKRGFYLAFQDIGACLAILSLRIYYKKCPAMVRNLAAFS |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ---------------------TRTIVWICLTLITGLVVLLLLLICKKRHCGYSKAFQDSD |
| EPH1j | ---------------------TRTIVWICLTLITGLVVLLLLLICKKRHCGYSKAFQDSD |
| EPH1k | ---------------------TRTIVWICLTLITGLVVLLLLLICKKRHCGYSKAFQDSD |
| EPH1l | ---------------------TRTIVWICLTLITGLVVLLLLLICKKRHCGYSKAFQDSD |
| EPH1m | ---------------------TRTIVWICLTLITGLVVLLLLLICKKRHCGYSKAFQDSD |
| EPH1n | ---------------------TRTIVWICLTLITGLVVLLLLLICKKRHCGYSKAFQDSD |
| EPH1o | ---------------------TRTIVWICLTLITGLVVLLLLLICKKRHCGYSKAFQDSD |
| EPH1p | GADLGVRRLKLNTEVRSVCPLSKRGFYLAFQDIGACLAILSLRIYYKKCPAMVRNLAAFS |
| EPH1q | GADLGVRRLKLNTEVRSVCPLSKRGFYLAFQDIGACLAILSLRIYYKKCPAMVRNLAAFS |
| EPH1r | GADLGVRRLKLNTEVRSVGPLSKRGFYLAFQDIGACLAILSLRIYYKKCPAMVRNLAAFS |
| EPH1a | EEKMHYQNGQAPPPVFLPLHHPPGK-LPEPQFYAEPHTYEEPGRAGRSFTR-EIEASRIH |
| EPH1b | EAVTGADSSSLVEVRGQCVRHSEERDTPKMYCSAEGEWLVPIGKCVCSAGYEERRDACVA |
| EPH1c | -------------------------------------------------------TRSIH |
| EPH1d | -------------------------------------------------------TRSIH |
| EPH1e | -------------------------------------------------------TRSIH |
| EPH1f | -------------------------------------------------------TRSIH |
| EPH1g | -------------------------------------------------------TRSIH |
| EPH1h | -------------------------------------------------------TRSIH |
| EPH1i | EEKMHYQNGQAPPPVFLPLHHPPGK-LPEPQFYAEPHTYEEPGPAGRSFTR-EIEASRIH |
| EPH1j | EEKMHYQNGQAPPPVFLPLHHPPGK-LPEPQFYAQPHTYEEPGRAGRSFTR-EIEASRIH |
| EPH1k | EEKMHYQNGQAPPPVFLPLHHPPGK-LPEPQFYAEPHTYEEPGRAGRSFTR-EIEASRIH |
| EPH1l | EEKMHYQNGQAPPPVFLPLHHPPGK-LPEPQFYAEPHTYEEPGRAGRSFTR-EIEASRIH |
| EPH1m | EEKMHYQNGQAPPPVFLPLHHPPGK-LPEPQFYAQPHTYEEPGRAGRSFTR-EIEASRIH |
| EPH1n | EEKMHYQNGQAPPPVFLPLHHPPGK-LPEPQFYAEPHTYEEPGRAGRSFTR-EIEASRIH |
| EPH1o | EEKMHYQNGQAPPPVFLPLHHPPGK-LPEPQFYAEPHTYEEPGRAGRSFTR-EIEASRIH |
| EPH1p | EAVTGADSSSLVEVRGQCVRHSEERDTPKMYCSAEGEWLVPIGKCVCSAGYEERRDACVA |
| EPH1q | EAVTGADSSSLVEVRGQCVRHSEERDTPKMYCSAEGEWLVPIGKCVCSAGYEERRDACVA |
| EPH1r | EAVTGADSSSLVEVRGQCVRHSEERDTPKMYCSAEGEWLVPIGKCVCSAGYEERRDACVA |
| EPH1a | IEKIIGSGDSGEVCYGRLRVPGQRDVPVAIKALKAGYTERQRRDFLSEASIMGQFDHPNI |
| EPH1b | CELGFYKSAPGDQLCARCPPHSHSAAPAAQACHCDLSYYRAALDPPSSACTRPPSAPVNL |
| EPH1c | IEKIIGSGDSGEVCYGRLRVPGQRDVPVAIKALKAGYTERQRRDFLSEASIMGQFDHPNI |
| EPH1d | IEKIIGSGDSGEVCYGRLRVPGQRDVPVAIKALKAGYTERQRRDFLSEASIMGQFDHPNI |
| EPH1e | IEKIIGSGDSGEVCYGRLRVPGQRDVPVAIKALKAGYTERQRRDFLSEASIMGQLDHPNI |
| EPH1f | IEKIIGSGDSGEVCYGRLRVPGQRDVPVAIKALKAGYTERQRRDFLSEASIMGQFDHPNI |
| EPH1g | IEKIIGSGDSGEVCYGRLRVPGQRDVPVAIKALKAGYTERQRRDFLSEASIMGQFDHPNI |
| EPH1h | IEKIIGSGDSGEVCYGRLRVPGQRDVPVAIKALKAGYTERQRRDFLSEASIMGQFDHPNI |
| EPH1i | IEKIIGSGDSGEVCYGRLRVPGQRDVPVAIKALKAGYTERQRRDFLSEASIMGQFDHPNI |
| EPH1j | IEKIIGSGDSGEVCYGRLRVPGQRDVPVAIKALKAGYTERQRRDFLSEASIMGQFDHPNI |
| EPH1k | IEKIIGSGDSGEVCYGRLRVPGQRDVPVAIKALKAGYTERQRRDFLSEASIMGQFDHPNI |
| EPH1l | IEKIIGSGDSGEVCYGRLRVPGQRDVPVAIKALKAGYTERQRRDFLSEASIMGQFDHPNI |
| EPH1m | IEKIIGSGDSGEVCYGRLRVPGQRDVPVAIKALKAGYTERQRRDFLSEASIMGQFDHPNI |
| EPH1n | IEKIIGSGDSGEVCYGRLRVPGQRDVPVAIKALKAGYTERQRRDFLSEASIMGQFDHPNI |
| EPH1o | IEKIIGSGDSGEVCYGRLRVPGQRDVPVAIKALKAGYTERQRRDFLSEASIMGQFDHPNI |
| EPH1p | CELGFYKSAPGDQLCARCPPHSHSAAPAAQACHCDLSYYRAALDPPSSACTRPPSAPVNL |
| EPH1q | CELGFYKSAPGDQLCARCPPHSHSAAPAAQACHCDLSYYRAALDPPSSACTRPPSAPVNL |
| EPH1r | CELGFYKSAPGDQLCARCPPHSHSAAPAAQACHCDLSYYRAALDPPSSACTRPPSAPVNL |
| EPH1a | IRLEG--VVTRGRLAMIVTEYMENGSLDTFLRTHDGQFTIMQLVGM-LRGVGAGMRYLSD |
| EPH1b | ISSVNGTSVTLEWAPPLDPGGRSDITYNAVCRRCPWALSRCEACGSGTRFVPQQTSLVQA |
| EPH1c | IRLEG--VVTRGRLAMIVTEYMENGSLDTFLRTHDGQFTIMQLVGM-LRGVGAGMRYLSD |
| EPH1d | IRLEG--VVTRGRLAMIVTEYMENGSLDTFLR---------------------------- |
| EPH1e | IRLEG--VVTRGRLAMIVTEYMENGSLDTFLR---------------------------- |
| EPH1f | IRLEG--VVTRGRLAMIVTEYMENVSLDTFLR---------------------------- |
| EPH1g | IRLEG--VVTRGRLAMIVTEYMENGSLDTFLR---------------------------- |
| EPH1h | IRLEG--VVTRGRLAMTVTEYMENVSLDTFLR---------------------------- |
| EPH1i | IRLEG--VVTRGRLAMIVTEYMENGSLDTFLRTHDGQFTIMQLVGM-LRGVGAGMRYLSD |
| EPH1j | IRLEG--VVTRGRLAMIVTEYMENGSLDTFLRTHDGQFTIMQLVGM-LRGVGAGMRYLSD |
| EPH1k | IRLEG--VVTRGRLAMIVTEYMENGSLDTFLRTHDGQFTIMQLVGM-LRGVGAGMRYLSD |
| EPH1l | IRLEG--VVTRGRLAMIVTEYMENGSLDTFLRTHDGQFTIMQLVGM-LRGVGAGMRYLSD |
| EPH1m | IRLEG--VVTRGRLAMIVTEYMENGSLDTFLRTHDGQFTIMQLVGM-LRGVGAGMRYLSD |
| EPH1n | IRLEG--VVTRGRLAMIVTEYMENGSLDTFLRTHDGQFTIMQLVGM-LRGVGAVMRYLSD |
| EPH1o | IRLEG--VVTRGRLAMIVTEYMENGSLDTFLRTHDGQFTIMQLVGM-LRGVGAGMRYLSD |
| EPH1p | ISSVNGTSVTLEWAPPLDPGGRSDITYNAVCRRCPWALSRCEACGSGTRFVPQQTSLVQA |
| EPH1q | ISSVNGTSVTLEWAPPLDPGGRSDITYNAVCRRCPWALSRCEACGSGTRFVPQQTSLVQA |
| EPH1r | ISSVNGTSVTLEWAPPLDPGGRSDITYNAVCRRCPWALSRCEACGSGTRFVPQQTSLVQA |
| EPH1a | LGYVHRDLAARNVLVDSNLVCKVSDFGLSRVLEDDPDAAYTTTGGKIPIRWTAPEAIAFR |
| EPH1b | SLLVANLLAHMNYSFWIEAVNGVSDLS----PEPRRAAVVNITTNQAAPSQVVVIRQERA |
| EPH1c | LGYVHRDLAARNVLVDSNLVCKVSDFGLSRVLEDDPDAAYTTTGGKIPIRWTAPEAIAFR |
| EPH1d | -------------------------------------------GGKIPIRWTAPEAIAFR |
| EPH1e | -------------------------------------------GGKIPIRWTAPEAIAFR |
| EPH1f | -------------------------------------------GGKIPIRWTAPEAIAFR |
| EPH1g | -------------------------------------------GGKIPIRWTAPEAIAFR |
| EPH1h | -------------------------------------------GGKIPIRWTAPEAIAFR |
| EPH1i | LGYVHRDLAARNVLVDSNLVCKVSDFGLSRVLEDDPDAAYTTTGGKIPIRWTAPEAIAFR |
| EPH1j | LGYVHRDLAARNVLVDSNLVCKVSDFGLSRVLEDDPDAAYTTTGGKIPIRWTAPEAIAFR |
| EPH1k | LGYVHRDLAARNVLVDSNLVCKVSDFGLSRVLEDDPDAAYTTTGGKIPIRWTAPEAIAFR |
| EPH1l | LGYVHRDLAARNVLVDSNLVCKVSDFGLSRVLEDDPDAAYTTTGGKIPIRWTAPEAIAFR |
| EPH1m | LGYVHRDLAARNVLVDSNLVCKVSDFGLSRVLEDDPDAAYTTTGGKIPIRWTAPEAIAFR |
| EPH1n | LGYVHRDLAARNVLVDSNLVCKVSDFGLSRVLEDDPDAAYTTTGGKIPIRWTAPEAIAFR |
| EPH1o | LGYVHRDLAARNVLVDSNLVCKVSDFGLSRVLEDDPDAAYTTTGGKIPIRWTAPEAIAFR |
| EPH1p | SLLVANLLAHMNYSFWIEAVNGVSDLS----PEPRRAAVVNITTNQAAPSQVVVIRQERA |
| EPH1q | SLLVANLLAHMNYSFWIEAVNGVSDLS----PEPRRAAVVNITTNQAAPSQVVVIRQERA |
| EPH1r | SLLVANLLAHMNYSFWIEAVNGVSDLS----PEPRRAAVVNITTNQAAPSQVVVIRQERA |
| EPH1a | TFSSASDVWSFGVVMWEVLAYGERPYWNMTNRDVISSVEEGYRLPAPMGCPHALHQLMLD |
| EPH1b | GQTSVSLLWQEPEQPNGIILEYEIKYYEKDKEMQSYSTLKAVTTRATVSGLKPGTRYVFQ |
| EPH1c | TFSSASDVWSFGVVMWEVLAYGERPYWNMTNRDVISSVEEGYRLPAPMGCPHALHQLMLD |
| EPH1d | TFSSASDVWSFGVVMWEVLAYGERPYWNMTNRDVISSVEEGYRLPAPMGCPHALHQLMLD |
| EPH1e | TFSSASDVWSFGVVMWEVLAYGERPYWNMTNRDVISSVEEGYRLPAPMGCPHALHQLMLD |
| EPH1f | TFSSASDVWSFGVVMWEVLAYGERPYWNMTNRDVISSVEEGYRLPAPMGCPHALHQLMLD |
| EPH1g | TFSSASDVWSFGVVMWEVLAYGERPYWNMTNRDVISSVEEGYRLPAPMGCPHALHQLMLD |
| EPH1h | TFSSASDVWSFGVVMWEVLAYGERPYWNMTNRDVISSVEEGYRLPAPMGCPHALHQLMLD |
| EPH1i | TFSSASDVWSFGVVMWEVLAYGERPYWNMTNRDVISSVEEGYRLPAPMGCPHALHQLMLD |
| EPH1j | TFSSASDVWSFGVVMWEVLAYGERPYWNMTNRDVISSVEEGYRLPAPMGCPHALHQLMLD |
| EPH1k | TFSSASDVWSFGVVMWEVLAYGERPYWNMTNRDVISSVEEGYRLPAPMGCPHALHQLMLD |
| EPH1l | TFSSASDVWSFGVVMWEVLAYGERPYWNMTNRDVISSVEEGYRLPAPMGCPHALHQLMLD |
| EPH1m | TFSSASDVWSFGVVMWEVLAYGERPYWNMTNRDVISSVEEGYRLPAPMGCPHALHQLMLD |
| EPH1n | TFSSASDVWSFGVVMWEVLAYGERPYWNMTNRDVISSVEEGYRLPAPMGCPHALHQLMLD |
| EPH1o | TFSSASDVWSFGVVMWEVLAYGERPYWNMTNRDVISSVEEGYRLPAPMGCPHALHQLMLD |
| EPH1p | GQTSVSLLWQEPEQPNGIILEYEIKYYEKDKEMQSYSTLKAVTTRATVSGLKPGTRYVFQ |
| EPH1q | GQTSVSLLWQEPEQPNGIILEYEIKYYEKDKEMQSYSTLKAVTTRATVSGLKPGTRYVFQ |
| EPH1r | GQTSVSLLWQEPEQPNGIILEYEIKYYEKDKEMQSYSTLKAVTTRATVSGLKPGTRYVFQ |
| EPH1a | CWHKDRAQRPRFSQIVSVLDALIRSPESLRATATVSRCPPPAFVRSCFDLRGGSGGGGGL |
| EPH1b | VRARTSAGCGRFSQAMEVETGKPRPRYDTRT----------------------------- |
| EPH1c | CWHKDRAQRPRFSQIVSVLDALIRSPESLRATATVSRCPPPAFVRSCFDLRGGSGGGGGL |
| EPH1d | CWHKDRAQRPRFSQIVSVLDALIRSPESLRATATVSRCPPPAFVRSCFDLRGGSGGGGGL |
| EPH1e | CWHKDRAQRPRFSQIVSVLDALIRSPESLRATATVSRCPPPAFVRSCFDLRGGSGGGGGL |
| EPH1f | CWHKDRAQRPRFSQIVSVLDALIRSPESLRATATVSRCPPPAFVRSCFDLRGGSGGGGGL |
| EPH1g | CWHKDRAQRPRFSQIVSVLDALIRSPESLRATATVSRCPPPAFVRSCFDLRGGSGGGGGL |
| EPH1h | CWHKDRAQRPRFSQIVSVLDALIRSPESLRATATVSRCPPPAFVRSCFDLRGGSGGGGGL |
| EPH1i | CWHKDRAQRPRFSQIVSVLDALIRSPESLRATATVSRCPPPAFVRSCFDLRGGSGGGGGL |
| EPH1j | CWHKDRAQRPRFSQIVSVLDALIRSPESLRATATVSRCPPPAFVRSCFDLRGGSGGGGGL |
| EPH1k | CWHKDRAQRPRFSQIVSVLDALIRSPESLRATATVSRCPPPAFVRSCFDLRGGSGGGGGL |
| EPH1l | CWHKDRAQRPRFSQIVKLGKGGRADPAFLYK----------------------------- |
| EPH1m | CWHKDRAQRPRFSQIVKLGKGGRA------------------------------------ |
| EPH1n | CWHKDRAQRPRFSQIVKLGKGGRATQLLVQVGY--------------------------- |
| EPH1o | CWHKDRAQRPRFSQIVKLG----------------------------------------- |
| EPH1p | VRARTSAGCGRFSQAMEVETGKPRPRYDTRT----------------------------- |
| EPH1q | VRARTPAGCGRFSQAMEVETGKPRPRYDTRTIVWICLTLITGLVVLLLLLICKKRHCGYS |
| EPH1r | VRARTSAGCGRFSQAMEVETGKPRPRYDTRTIVWICLTLITGLVVLLLLLICKKRHCGYS |
| EPH1a | TVGDWLDSIRMGRYRDHFAAGGYSSLGMVLRMNAQDVRALGITLMGHQKKILGSIQTMRA |
| EPH1b | ------------------------------------------------------------ |
| EPH1c | TVGDWLDSIRMGRYRDHFAAGGYSSLGMVLRMNAQDVRALGITLMGHQKKILGSIQTMRA |
| EPH1d | TVGDWLDSIRMGRYRDHFAAGGYSSLGMVLRMNAQDVRALGIALMGHQKKILGSIQTMRA |
| EPH1e | TVGDWLDSIRMGRYRDHFAAGGYSSLGMVLRMNAQDVRALGITLMGHQKKILGSIQTMRA |
| EPH1f | TVGDWLDSIRMGRYRDHFAAGGYSSLGMVLRMNAQDVRALGITLMGHQKKILGSIQTMRA |
| EPH1g | TVGDWLDSIRMGRYRDHFAAGGYSSLGMVLRMNAQDVRALGITLMGHQKKILGSIQTMRA |
| EPH1h | TVGDWLDSIRMGRYRDHFAAGGYSSLGMVLRMNAQDVRALGITLMGHQKKILGSIQTMRA |
| EPH1i | TVGDWLDSIRMGRYRDHFAAGGYSSLGMVLRMNAQDVRALGITLMGHQKKILGSIQTMRA |
| EPH1j | TVGDWLDSIRMGRYRDHFAAGGYSSLGMVLRMNAQDVRALGITLMGHQKKILGSIQTMRA |
| EPH1k | TVGDWLDSIRMGRYRDHFAAGGYSSLGMVLRMNAQDVRALGITLMGHQKKILGSIQTMRA |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | KAFQDSDEEKMHYQNGQAPPPVFLPLHHPPGKLPEPQFYAEPHTYEEPGRAGRSFTREIE |
| EPH1r | KAFQDSDEEKMHYQNGQAPPPVFLPLHHPPGKLPEPQFYAEPHTYEEPGRAGRSFTREIE |
| EPH1a | QLTSTQGPRRHL------------------------------------------------ |
| EPH1b | ------------------------------------------------------------ |
| EPH1c | QLTSTQGPRRHL------------------------------------------------ |
| EPH1d | QLTSTQGPRRHL------------------------------------------------ |
| EPH1e | QLTSTQGPRRHL------------------------------------------------ |
| EPH1f | QLTSTQGPRRHL------------------------------------------------ |
| EPH1g | QLTSTQGPRRHL------------------------------------------------ |
| EPH1h | QLTSTQGPRRHL------------------------------------------------ |
| EPH1i | QLTSTQGPRRHL------------------------------------------------ |
| EPH1j | QLTSTQGPRRHL------------------------------------------------ |
| EPH1k | QLTSTQGPRRHL------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | ASRIHIEKIIGSGDSGEVCYGRLRVPGQRDVPVAIKALKAGYTERQRRDFLSEASIMGQF |
| EPH1r | ASRIHIEKIIGSGDSGEVCYGRLRVPGQRDVPVAIKALKAGYTERQRRDFLSEASIMGQF |
| EPH1a | ------------------------------------------------------------ |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | DHPNIIRLEGVVTRGRLAMIVTEYMENGSLDTFLRGGKIPIRWTAPEAIAFRTFSSASDV |
| EPH1r | DHPNIIRLEGVVTRGRLAMIVTEYMENGSLDTFLRGGKIPIRWTAPEAIAFRTFSSASDV |
| EPH1a | ------------------------------------------------------------ |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | WSFGVVNWEVLAYGERPYWNMTNRDVISSVEEGYRLPAPMGCPHALHQLMLDCWHKDRAQ |
| EPH1r | WSFGVVMWEVLAYGERPYWNMTNRDVISSVEEGYRLPAPMGCPHALHQLMLDCWHKDRAQ |
| EPH1a | ------------------------------------------------------------ |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPH1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | RPRFSQIVSVLDALIRSPESLRATATVSRCPPPAFVRSCFDLRGGSGGGGGLTVGDWLDS |
| EPH1r | RPRFSQIVSVLDALIRSPESLRATATVSRCPPPAFVRSCFDLRGGSGGGGGLTVGDWLDS |
| EPH1a | ------------------------------------------------------------ |
| EPH1b | ------------------------------------------------------------ |
| EPH1c | ------------------------------------------------------------ |
| EPH1d | ------------------------------------------------------------ |
| EPH1e | ------------------------------------------------------------ |
| EPH1f | ------------------------------------------------------------ |
| EPH1g | ------------------------------------------------------------ |
| EPH1h | ------------------------------------------------------------ |
| EPE1i | ------------------------------------------------------------ |
| EPH1j | ------------------------------------------------------------ |
| EPH1k | ------------------------------------------------------------ |
| EPH1l | ------------------------------------------------------------ |
| EPH1m | ------------------------------------------------------------ |
| EPH1n | ------------------------------------------------------------ |
| EPH1o | ------------------------------------------------------------ |
| EPH1p | ------------------------------------------------------------ |
| EPH1q | IRMGRYRDHFAAGGYSSLGMVLRMNAQDVRALGITLMGHQKKILGSIQTMRAQLTSTQGP |
| EPH1r | IRMGRYRDHFAAGGYSSLGMVLRMNAQDVRALGITLMGHQKKILGSIQTMRAQLTSTQGP |
| EPH1a | ---- |
| EPH1b | ---- |
| EPH1c | ---- |
| EPH1d | ---- |
| EPH1e | ---- |
| EPH1f | ---- |
| EPH1g | ---- |
| EPH1h | ---- |
| EPH1i | ---- |
| EPH1j | ---- |
| EPH1k | ---- |
| EPH1l | ---- |
| EPH1m | ---- |
| EPH1n | ---- |
| EPH1o | ---- |
| EPH1p | ---- |
| EPH1q | RRHL |
| EPH1r | RRHL |
| EPH1a | (SEQ ID NO: 2) |
| EPH1b | (SEQ ID NO: 4) |
| EPH1c | (SEQ ID NO: 6) |
| EPH1d | (SEQ ID NO: 8) |
| EPH1e | (SEQ ID NO: 10) |
| EPH1f | (SEQ ID NO: 12) |
| EPH1g | (SEQ ID NO: 14) |
| EPH1h | (SEQ ID NO: 16) |
| EPH1i | (SEQ ID NO: 18) |
| EPH1j | (SEQ ID NO: 20) |
| EPH1k | (SEQ ID NO: 22) |
| EPH1l | (SEQ ID NO: 24) |
| EPH1m | (SEQ ID NO: 26) |
| EPH1n | (SEQ ID NO: 28) |
| EPE1o | (SEQ ID NO: 30) |
| EPH1p | (SEQ ID NO: 32) |
| EPH1q | (SEQ ID NO: 34) |
| EPH1r | (SEQ ID NO: 36) |

Further analysis of the EPH1a protein yielded the following properties shown in Table 1C.

TABLE 1C
Protein Sequence Properties EPH1a

| | |
|---|---|
| SignalP analysis: | Cleavage site between residues 28 and 29 |
| PSORT II analysis: | |

| | |
|---|---|
| PSG: | a new signal peptide prediction method |
| | N-region: length 7; pos. chg 2; neg. chg 0 |
| | H-region: length 21; peak value 8.31 |
| | PSG score: 3.91 |
| GvH: | von Heijne's method for signal seq. recognition |
| | GvH score (threshold: −2.1): −1.16 |
| | possible cleavage site: between 30 and 31 |

>>> Seems to have a cleavable signal peptide (1 to 30)

| | |
|---|---|
| ALOM: | Klein et al's method for TM region allocation |
| | Init position for calculation: 31 |
| | Tentative number of TMS(s) for the threshold 0.5: 2 |
| | Number of TMS(s) for threshold 0.5: 1 |
| | INTEGRAL Likelihood = −13.85 Transmembrane 546-562 |
| | PERIPHERAL Likelihood = 2.49 (at 390) |
| | ALOM score: −13.85 (number of TMSs: 1) |
| MTOP: | Prediction of membrane topology (Hartmann et al.) |
| | Center position for calculation: 15 |
| | Charge difference: −4.5 C(−1.5) - N(3.0) |
| | N >= C: N-terminal side will be inside |

>>>membrane topology: type 1a (cytoplasmic tail 563 to 1005)

| | |
|---|---|
| MITDISC: | discrimination of mitochondrial targeting seq |

| | |
|---|---|
| R content: 3 | Hyd Moment(75): 2.64 |
| Hyd Moment(95): 2.19 | G content: 2 |
| D/E content: 1 | S/T content: 3 |
| Score: −3.24 | |

| | |
|---|---|
| Gavel: | prediction of cleavage sites for mitochondrial preseq |
| | R-2 motif at 39 ARG\|EV |
| NUCDISC: | discrimination of nuclear localization signals |
| | pat4: KKRH (3) at 564 |
| | pat7: none |
| | bipartite: none |
| | content of basic residues: 10.9% |
| | NLS Score: −0.29 |
| KDEL: | ER retention motif in the C-terminus: none |

ER Membrane Retention Signals:
XXRR-like motif in the N-terminus: APAR none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23);
55.6%: endoplasmic reticulum
22.2%: Golgi
11.1%: plasma membrane
11.1%: extracellular, including cell wall
>> prediction for CG54020-01 is end (k = 9)

TABLE 1D
Geneseq Results for EPH1a

| | | | Identities/ | |
|---|---|---|---|---|
| | | | Similari- | |
| | | EPH1a | ities | |
| | Protein/ | Residues/ | for the | |
| Geneseq | Organism/Length | Match | Matched | Expect |
| Identifier | [Patent #, Date] | Residues | Region | Value |
| ABP69349 | Human polypeptide | 1 . . . 1005 | 1004/1005 | 0.0 |
| | SEQ ID NO 1396- | 1 . . . 1005 | (99%) | |
| | Homo sapiens, | | 1005/1005 | |
| | 1005 aa. | | (99%) | |
| | [WO200270539-A2, | | | |
| | 12 SEP. 2002] | | | |
| AAE04362 | Human kinase | 1 . . . 1005 | 1005/1012 | 0.0 |
| | (PKIN)-3 - | 1 . . . 1012 | (99%) | |
| | Homo sapiens, | | 1005/1012 | |
| | 1012 aa. | | (99%) | |
| | [WO200146397-A2, | | | |
| | 28 JUN. 2001] | | | |
| AAE23799 | Ephrin type-A | 1 . . . 992 | 992/992 | 0.0 |
| | receptor 8-like | 1 . . . 992 | (100%) | |
| | (NOV2) protein - | | 992/992 | |
| | Unidentified, | | (100%) | |
| | 992 aa. | | | |
| | [WO200230979-A2, | | | |
| | 18 APR. 2002] | | | |
| AAU00691 | Ephrin type-A | 1 . . . 992 | 992/992 | 0.0 |
| | receptor 8-like | 1 . . . 992 | (100%) | |
| | protein - Homo | | 992/992 | |
| | sapiens, 992 aa. | | (100%) | |
| | [WO200129217-A2, | | | |
| | 26 APR. 2001] | | | |
| AAR85090 | EPH-like receptor | 12 . . . 1001 | 585/994 | 0.0 |
| | protein tyrosine | 16 . . . 990 | (58%) | |
| | kinase HEK7 - | | 746/994 | |
| | Homo sapiens, | | (74%) | |
| | 991 aa. | | | |
| | [WO9528484-A1, | | | |
| | 26 OCT. 1995] | | | |

In a BLAST search of public sequence databases, the EPH1 a protein was found to have homology to the proteins shown in the BLASTP data in Table 1E.

TABLE 1E
Public BLASTP Results for EPH1a

| | | | Identities/ | |
|---|---|---|---|---|
| | | | Similiari- | |
| | | EPH1a | ties | |
| Protein | | Residues/ | for the | |
| Accession | Protein/ | Match | Matched | Expect |
| Number | Organism/Length | Residues | Portion | Value |
| P29322 | Ephrin type-A | 1 . . . 1005 | 1005/1005 | 0.0 |
| | receptor 8 precursor | 1 . . . 1005 | (100%) | |
| | (EC 2.7.1.112) | | 1005/1005 | |
| | (Tyrosine-protein | | (100%) | |
| | kinase receptor EEK) | | | |
| | (EPH-and ELK- | | | |
| | related kinase) | | | |
| | (HEK3) - Homo | | | |
| | sapiens (Human), | | | |
| | 1005 aa. | | | |
| CAC38718 | Sequence 4 from | 1 . . . 992 | 992/992 | 0.0 |
| | Patent WO0129217 - | 1 . . . 992 | (100%) | |
| | Homo sapiens | | 992/992 | |
| | (Human), 992 aa | | (100%) | |
| | (fragment). | | | |
| O09127 | Ephrin type-A | 1 . . . 1005 | 956/1005 | 0.0 |
| | receptor 8 precursor | 1 . . . 1004 | (95%) | |
| | (EC 2.7.1.112) | | 976/1005 | |
| | (Tyrosine-protein | | (96%) | |
| | kinase receptor EEK) | | | |
| | (EPH-and ELK-related | | | |
| | kinase) - Mus | | | |
| | musculus (Mouse), | | | |
| | 1004 aa. | | | |
| 178843 | receptor protein- | 12 . . . 1001 | 585/994 | 0.0 |
| | tyrosine kinase - | 16 . . . 990 | (58%) | |
| | human, 991 aa | | 746/994 | |
| | (fragment). | | (74%) | |
| P54756 | Ephrin type-A | 12 . . . 1001 | 586/1016 | 0.0 |
| | receptor 5 precursor | 40 . . . 1036 | (57%) | |
| | (EC 2.7.1.112) | | 747/1016 | |
| | (Tyrosine-protein | | (72%) | |
| | kinase receptor | | | |
| | EHK-1) (Eph | | | |
| | homology kinase-1) | | | |
| | (Receptor | | | |
| | protein-tyrosine kinase | | | |
| | HEK7) - Homo | | | |
| | sapiens (Human), | | | |
| | 1037 aa. | | | |

PFam analysis predicts that the EPH1 a protein contains the domains shown in the Table 1F.

TABLE 1F
Domain Analysis of EPH1a

| | | Identities/ | |
|---|---|---|---|
| | EPH1a | Similarities | Expect |
| Pfam Domain | Match Region | for the Matched Region | Value |
| EPH_lbd | 31 . . . 204 | 123/177 (69%) | 4.7e−135 |
| | | 169/177 (95%) | |
| fn3 | 329 . . . 425 | 27/98 (28%) | 1.5e−07 |
| | | 67/98 (68%) | |
| fn3 | 437 . . . 524 | 30/90 (3 3%) | 9.6e−20 |
| | | 74/90 (82%) | |
| pkinase | 635 . . . 892 | 85/301 (28%) | 7.9e−74 |
| | | 199/301 (66%) | |
| SAM | 928 . . . 992 | 26/68 (38%) | 2.3e−21 |
| | | 55/68 (81%) | |

Anti-EPH-X Antibodies

Included in the invention are antibodies to EPH-X proteins, or fragments of EPH-X proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule.

Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO: 2n, wherein n is an integer between 1 and 18, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of EPH-X that is located on the surface of the protein, e.g., a hydrophilic region. A hydrophobicity analysis of the human EPH-X protein sequence indicates which regions of a EPH-X polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A EPH-X polypeptide or a fragment thereof comprises at least one antigenic epitope. An anti-EPH-X antibody of the present invention is said to specifically bind to antigen EPH-X when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No.8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that specifically bind to the immunizing agent. Alternatively, the lymphocytes can be immunized in vitro.

The immunizing agent typically includes the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically includes hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody comprises substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also comprises at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,( Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain. In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g. an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

EPH-X Nucleic Acids and Polypeptides

One aspect of the invention pertains to isolated nucleic acid molecules that encode EPH-X polypeptides or biologically active portions thereof. Also included in the invention are nucleic acid fragments sufficient for use as hybridization probes to identify EPH-X-encoding nucleic acids (e.g., EPH-X mRNA's) and fragments for use as PCR primers for the amplification and/or mutation of EPH-X nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A EPH-X nucleic acid can encode a mature EPH-X polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated EPH-X nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NO: 2n−1, wherein n is an integer between 1-18, or a complement of this aforementioned nucleotide sequence, can be isolated using standard molecular biology techniques and the sequence information provided herein. Using all or a portion of the nucleic acid sequence of SEQ ID NO: 2n−1, wherein n is an integer between 1-18, as a hybridization probe, EPH-X molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to EPH-X nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO: 2n−1, wherein n is an integer between 1-18, or a complement thereof. Oligonucleotides may be chemically synthesized and may also be used as probes.

In another embodiment, an isolated nucleic acid molecule of the invention comprises a nucleic acid molecule that is a complement of the nucleotide sequence SEQ ID NO: 2n−1, wherein n is an integer between 1-18, or a portion of this nucleotide sequence (e.g., a fragment that can be used as a probe or primer or a fragment encoding a biologically-active portion of a EPH-X polypeptide). A nucleic acid molecule that is complementary to the nucleotide sequence of SEQ ID NO: 2n−1, wherein n is an integer between 1-18, is one that is sufficiently complementary to the nucleotide sequence of SEQ ID NO: 2n−1, wherein n is an integer between 1-18, that it can hydrogen bond with little or no mismatches to the nucleotide sequence of SEQ ID NO: 2n−1, wherein n is an integer between 1-18, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

A full-length EPH-X clone is identified as containing an ATG translation start codon and an in-frame stop codon. Any disclosed EPH-X nucleotide sequence lacking an ATG start codon therefore encodes a truncated C-terminal fragment of the respective EPH-X polypeptide, and requires that the corresponding full-length cDNA extend in the 5′ direction of the disclosed sequence. Any disclosed EPH-X nucleotide sequence lacking an in-frame stop codon similarly encodes a truncated N-terminal fragment of the respective EPH-X polypeptide, and requires that the corresponding full-length cDNA extend in the 3′ direction of the disclosed sequence.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of EPH-X polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a EPH-X polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human EPH-X protein. Homologous nucleic acid sequences include those nucleic acid sequences that encode conservative amino acid substitutions (see below) in SEQ ID NO: 2n−1, wherein n is an integer between 1-18, as well as a polypeptide possessing EPH-X biological activity. Various biological activities of the EPH-X proteins are described below.

A EPH-X polypeptide is encoded by the open reading frame (“ORF”) of a EPH-X nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human EPH-X genes allows for the generation of probes and primers designed for use in identifying and/or cloning EPH-X homologues in other cell types, e.g. from other tissues, as well as EPH-X homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO: 2n−1, wherein n is an integer between 1-18; or an anti-sense strand nucleotide sequence of SEQ ID NO: 2n−1, wherein n is an integer between 1-18; or of a naturally occurring mutant of SEQ ID NO: 2n−1, wherein n is an integer between 1-18.

Probes based on the human EPH-X nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express a EPH-X protein, such as by measuring a level of a EPH-X-encoding nucleic acid in a sample of cells from a subject e.g., detecting EPH-X mRNA levels or determining whether a genomic EPH-X gene has been mutated or deleted.

“A polypeptide having a biologically-active portion of a EPH-X polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of EPH-X” can be prepared by isolating a portion of SEQ ID NO: 2n−1, wherein n is an integer between 1-18, that encodes a polypeptide having a EPH-X biological activity (the biological activities of the EPH-X proteins are described below), expressing the encoded portion of EPH-X protein (e.g., by recombinant expression in vitro) and assessing the activity of the encoded portion of EPH-X.

EPH-X Nucleic Acid and Polypeptide Variants

The invention further encompasses nucleic acid molecules that differ from the nucleotide sequences of SEQ ID NO: 2n−1, wherein n is an integer between 1-18, due to degeneracy of the genetic code and thus encode the same EPH-X proteins as that encoded by the nucleotide sequences of SEQ ID NO: 2n−1, wherein n is an integer between 1-18. In another embodiment, an isolated nucleic acid molecule of the invention has a nucleotide sequence encoding a protein having an amino acid sequence of SEQ ID NO: 2n, wherein n is an integer between 1-18.

In addition to the human EPH-X nucleotide sequences of SEQ ID NO: 2n−1, wherein n is an integer between 1-18, it will be appreciated by those skilled in the art that DNA sequence polymorphisms that lead to changes in the amino acid sequences of the EPH-X polypeptides may exist within a population (e.g., the human population). Such genetic polymorphism in the EPH-X genes may exist among individuals within a population due to natural allelic variation. As used herein, the terms “gene” and “recombinant gene” refer to nucleic acid molecules comprising an open reading frame (ORF) encoding a EPH-X protein, preferably a vertebrate EPH-X protein. Such natural allelic variations can typically result in 1-5% variance in the nucleotide sequence of the EPH-X genes. Any and all such nucleotide variations and resulting amino acid polymorphisms in the EPH-X polypeptides, which are the result of natural allelic variation and that do not alter the functional activity of the EPH-X polypeptides, are intended to be within the scope of the invention.

Moreover, nucleic acid molecules encoding EPH-X proteins from other species, and thus that have a nucleotide sequence that differs from any one of the human SEQ ID NO: 2n−1, wherein n is an integer between 1-18, are intended to be within the scope of the invention. Nucleic acid molecules corresponding to natural allelic variants and homologues of the EPH-X cDNAs of the invention can be isolated based on their homology to the human EPH-X nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 2n−1, wherein n is an integer between 1-18. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding EPH-X proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to any one of the sequences of SEQ ID NO: 2n−1, wherein n is an integer between 1-18, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 2n−1, wherein n is an integer between 1-18, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO: 2n−1, wherein n is an integer between 1-18, or fragments, analogs or derivatives thereof, under conditions of low stringency, is provided. A non-limiting example of low stringency hybridization conditions are hybridization in 35% formamide, 5×SSC, 50 mM Tris-HCI (pH 7.5), 5 mM EDTA, 0.02% PVP, 0.02% Ficoll, 0.2% BSA, 100 mg/ml denatured salmon sperm DNA, 10% (wt/volt) dextran sulfate at 40° C., followed by one or more washes in 2×SSC, 25 mM Tris-HCt (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of EPH-X sequences that may exist in the population, the skilled artisan will further appreciate that changes can be introduced by mutation into the nucleotide sequences of SEQ ID NO: 2n−1, wherein n is an integer between 1-18, thereby leading to changes in the amino acid sequences of the encoded EPH-X proteins, without altering the functional ability of said EPH-X proteins. For example, nucleotide substitutions leading to amino acid substitutions at “non-essential” amino acid residues can be made in the sequence of SEQ ID NO: 2n, wherein n is an integer between 1-18. A “non-essential” amino acid residue is a residue that can be altered from the wild-type sequences of the EPH-X proteins without altering their biological activity, whereas an “essential” amino acid residue is required for such biological activity. For example, amino acid residues that are conserved among the EPH-X proteins of the invention are particularly non-amenable to alteration. Amino acids for which conservative substitutions can be made are well-known within the art.

Another aspect of the invention pertains to nucleic acid molecules encoding EPH-X proteins that contain changes in amino acid residues that are not essential for activity. Such EPH-X proteins differ in amino acid sequence from any one of SEQ ID NO: 2n−1, wherein n is an integer between 1-18, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NO: 2n, wherein n is an integer between 1-18. Preferably, the protein encoded by the nucleic acid molecule is at least about 60% homologous to SEQ ID NO: 2n, wherein n is an integer between 1-18; more preferably at least about 70% homologous to SEQ ID NO: 2n, wherein n is an integer between 1-18; still more preferably at least about 80% homologous to SEQ ID NO: 2n, wherein n is an integer between 1-18; even more preferably at least about 90% homologous to SEQ ID NO: 2n, wherein n is an integer between 1-18; and most preferably at least about 95% homologous to SEQ ID NO: 2n, wherein n is an integer between 1-18.

An isolated nucleic acid molecule encoding a EPH-X protein homologous to the protein of SEQ ID NO: 2n, wherein n is an integer between 1-18, can be created by introducing one or more nucleotide substitutions, additions or deletions into the nucleotide sequence of SEQ ID NO: 2n−1, wherein n is an integer between 1-18, such that one or more amino acid substitutions, additions or deletions are introduced into the encoded protein.

Mutations can be introduced into any of SEQ ID NO: 2n−1, wherein n is an integer between 1-18, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the EPH-X protein is replaced with another amino acid residue from the same side chain family. Alternatively, in another embodiment, mutations can be introduced randomly along all or part of a EPH-X coding sequence, such as by saturation mutagenesis, and the resultant mutants can be screened for EPH-X biological activity to identify mutants that retain activity. Following mutagenesis of any one of SEQ ID NO: 2n−1, wherein n is an integer between 1-18, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant EPH-X protein can be assayed for (i) the ability to form protein:protein interactions with other EPH-X proteins, other cell-surface proteins, or biologically-active portions thereof, (ii) complex formation between a mutant EPH-X protein and a EPH-X ligand; or (iii) the ability of a mutant EPH-X protein to bind to an intracellular target protein or biologically-active portion thereof, (e.g. avidin proteins).

In yet another embodiment, a mutant EPH-X protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO: 2n−1, wherein n is an integer between 1-18, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire EPH-X coding strand, or to only a portion thereof. Nucleic acid molecules encoding fragments, homologs, derivatives and analogs of a EPH-X protein of SEQ ID NO: 2n, wherein n is an integer between 1-18, or antisense nucleic acids complementary to a EPH-X nucleic acid sequence of SEQ ID NO: 2n−1, wherein n is an integer between 1-18, are additionally provided.

In one embodiment, an antisense nucleic acid molecule is antisense to a “coding region” of the coding strand of a nucleotide sequence encoding a EPH-X protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the EPH-X protein. The term “noncoding region” refers to 5′ and 3′ sequences which flank the coding region that are not translated into amino acids (i.e., also referred to as 5′ and 3′ untranslated regions).

Given the coding strand sequences encoding the EPH-X protein disclosed herein, antisense nucleic acids of the invention can be designed according to the rules of Watson and Crick or Hoogsteen base pairing. The antisense nucleic acid molecule can be complementary to the entire coding region of EPH-X mRNA, but more preferably is an oligonucleotide that is antisense to only a portion of the coding or noncoding region of EPH-X mRNA. For example, the antisense oligonucleotide can be complementary to the region surrounding the translation start site of EPH-X mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a EPH-X protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave EPH-X mRNA transcripts to thereby inhibit translation of EPH-X mRNA. A ribozyme having specificity for a EPH-X-encoding nucleic acid can be designed based upon the nucleotide sequence of a EPH-X cDNA disclosed herein (i.e., any one of SEQ ID NO: 2n−1, wherein n is an integer between 1-18). For example, a derivative of a Tetrahymena L-19 IVS RNA can be constructed in which the nucleotide sequence of the active site is complementary to the nucleotide sequence to be cleaved in a EPH-X-encoding mRNA. See, e.g., U.S. Pat. No. 4,987,071 to Cech, et al. and U.S. Pat. No. 5,116,742 to Cech, et al. EPH-X mRNA can also be used to select a catalytic RNA having a specific ribonuclease activity from a pool of RNA molecules. See, e.g., Bartel et al., (1993) Science 261:1411-1418.

Alternatively, EPH-X gene expression can be inhibited by targeting nucleotide sequences complementary to the regulatory region of the EPH-X nucleic acid (e.g., the EPH-X promoter and/or enhancers) to form triple helical structures that prevent transcription of the EPH-X gene in target cells. See, e.g., Helene, 1991. Anticancer Drug Des. 6: 569-84; Helene, et al. 1992. Ann. N. Y. Acad. Sci. 660: 27-36; Maher, 1992. Bioassays 14: 807-15.

In various embodiments, the EPH-X nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of EPH-X can be used in therapeutic and diagnostic applications. For example, PNAs can be used as antisense or antigene agents for sequence-specific modulation of gene expression by, e.g., inducing transcription or translation arrest or inhibiting replication. PNAs of EPH-X can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of EPH-X can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of EPH-X can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

EPH-X Polypeptides

A polypeptide according to the invention includes a polypeptide including the amino acid sequence of EPH-X polypeptides whose sequences are provided in any one of SEQ ID NO: 2n, wherein n is an integer between 1-18. The invention also includes a mutant or variant protein any of whose residues may be changed from the corresponding residues shown in any one of SEQ ID NO: 2n, wherein n is an integer between 1-18, while still encoding a protein that maintains its EPH-X activities and physiological functions, or a functional fragment thereof.

In general, a EPH-X variant that preserves EPH-X-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated EPH-X proteins, and biologically-active portions thereof, or derivatives, fragments, analogs or homologs thereof. Also provided are polypeptide fragments suitable for use as immunogens to raise anti-EPH-X antibodies. In one embodiment, native EPH-X proteins can be isolated from cells or tissue sources by an appropriate purification scheme using standard protein purification techniques. In another embodiment, EPH-X proteins are produced by recombinant DNA techniques. Alternative to recombinant expression, a EPH-X protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the EPH-X protein is derived, or substantially free from chemical precursors or other chemicals when chemically synthesized. The language “substantially free of cellular material” includes preparations of EPH-X proteins in which the protein is separated from cellular components of the cells from which it is isolated or recombinantly-produced. In one embodiment, the language “substantially free of cellular material” includes preparations of EPH-X proteins having less than about 30% (by dry weight) of non-EPH-X proteins (also referred to herein as a “contaminating protein”), more preferably less than about 20% of non-EPH-X proteins, still more preferably less than about 10% of non-EPH-X proteins, and most preferably less than about 5% of non-EPH-X proteins. When the EPH-X protein or biologically-active portion thereof is recombinantly-produced, it is also preferably substantially free of culture medium, i.e., culture medium represents less than about 20%, more preferably less than about 10%, and most preferably less than about 5% of the volume of the EPH-X protein preparation.

The language “substantially free of chemical precursors or other chemicals” includes preparations of EPH-X proteins in which the protein is separated from chemical precursors or other chemicals that are involved in the synthesis of the protein. In one embodiment, the language “substantially free of chemical precursors or other chemicals” includes preparations of EPH-X proteins having less than about 30% (by dry weight) of chemical precursors or non-EPH-X chemicals, more preferably less than about 20% chemical precursors or non-EPH-X chemicals, still more preferably less than about 10% chemical precursors or non-EPH-X chemicals, and most preferably less than about 5% chemical precursors or non-EPH-X chemicals.

Biologically-active portions of EPH-X proteins include peptides comprising amino acid sequences sufficiently homologous to or derived from the amino acid sequences of the EPH-X proteins (e.g., the amino acid sequence of SEQ ID NO: 2n, wherein n is an integer between 1-18) that include fewer amino acids than the full-length EPH-X proteins, and exhibit at least one activity of a EPH-X protein. Typically, biologically-active portions comprise a domain or motif with at least one activity of the EPH-X protein. A biologically-active portion of a EPH-X protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native EPH-X protein.

In an embodiment, the EPH-X protein has an amino acid sequence of SEQ ID NO: 2n, wherein n is an integer between 1-18. In other embodiments, the EPH-X protein is substantially homologous to SEQ ID NO: 2n, wherein n is an integer between 1-18, and retains the functional activity of the protein of SEQ ID NO: 2n, wherein n is an integer between 1-18, yet differs in amino acid sequence due to natural allelic variation or mutagenesis, as described in detail, below. Accordingly, in another embodiment, the EPH-X protein is a protein that comprises an amino acid sequence at least about 45% homologous to the amino acid sequence of SEQ ID NO: 2n, wherein n is an integer between 1-18, and retains the functional activity of the EPH-X proteins of SEQ ID NO: 2n, wherein n is an integer between 1-18.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO: 2n−1, wherein n is an integer between 1-18.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides EPH-X chimeric or fusion proteins. As used herein, a EPH-X “chimeric protein” or “fusion protein” comprises a EPH-X polypeptide operatively-linked to a non-EPH-X polypeptide. An “EPH-X polypeptide” refers to a polypeptide having an amino acid sequence corresponding to a EPH-X protein of SEQ ID NO: 2n, wherein n is an integer between 1-18, whereas a “non-EPH-X polypeptide” refers to a polypeptide having an amino acid sequence corresponding to a protein that is not substantially homologous to the EPH-X protein, e.g., a protein that is different from the EPH-X protein and that is derived from the same or a different organism. Within a EPH-X fusion protein the EPH-X polypeptide can correspond to all or a portion of a EPH-X protein. In one embodiment, a EPH-X fusion protein comprises at least one biologically-active portion of a EPH-X protein. In another embodiment, a EPH-X fusion protein comprises at least two biologically-active portions of a EPH-X protein. In yet another embodiment, a EPH-X fusion protein comprises at least three biologically-active portions of a EPH-X protein. Within the fusion protein, the term “operatively-linked” is intended to indicate that the EPH-X polypeptide and the non-EPH-X polypeptide are fused in-frame with one another. The non-EPH-X polypeptide can be fused to the N-terminus or C-terminus of the EPH-X polypeptide.

In one embodiment, the fusion protein is a GST-EPH-X fusion protein in which the EPH-X sequences are fused to the C-terminus of the GST (glutathione S-transferase) sequences. Such fusion proteins can facilitate the purification of recombinant EPH-X polypeptides.

In another embodiment, the fusion protein is a EPH-X protein containing a heterologous signal sequence at its N-terminus. In certain host cells (e.g., mammalian host cells), expression and/or secretion of EPH-X can be increased through use of a heterologous signal sequence.

In yet another embodiment, the fusion protein is a EPH-X-immunoglobulin fusion protein in which the EPH-X sequences are fused to sequences derived from a member of the immunoglobulin protein family. The EPH-X-immunoglobulin fusion proteins of the invention can be incorporated into pharmaceutical compositions and administered to a subject to inhibit an interaction between a EPH-X ligand and a EPH-X protein on the surface of a cell, to thereby suppress EPH-X-mediated signal transduction in vivo. The EPH-X-immunoglobulin fusion proteins can be used to affect the bioavailability of a EPH-X cognate ligand. Inhibition of the EPH-X ligand/EPH-X interaction may be useful therapeutically for both the treatment of proliferative and differentiative disorders, as well as modulating (e.g. promoting or inhibiting) cell survival. Moreover, the EPH-X-immunoglobulin fusion proteins of the invention can be used as immunogens to produce anti-EPH-X antibodies in a subject, to purify EPH-X ligands, and in screening assays to identify molecules that inhibit the interaction of EPH-X with a EPH-X ligand.

A EPH-X chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A EPH-X-encoding nucleic acid can be cloned into such an expression vector such that the fusion moiety is linked in-frame to the EPH-X protein.

EPH-X Agonists and Antagonists

The invention also pertains to variants of the EPH-X proteins that function as either EPH-X agonists (i.e., mimetics) or as EPH-X antagonists. Variants of the EPH-X protein can be generated by mutagenesis (e.g., discrete point mutation or truncation of the EPH-X protein). An agonist of the EPH-X protein can retain substantially the same, or a subset of, the biological activities of the naturally occurring form of the EPH-X protein. An antagonist of the EPH-X protein can inhibit one or more of the activities of the naturally occurring form of the EPH-X protein by, for example, competitively binding to a downstream or upstream member of a cellular signaling cascade which includes the EPH-X protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the EPH-X proteins.

Variants of the EPH-X proteins that function as either EPH-X agonists (i.e., mimetics) or as EPH-X antagonists can be identified by screening combinatorial libraries of mutants (e.g., truncation mutants) of the EPH-X proteins for EPH-X protein agonist or antagonist activity. In one embodiment, a variegated library of EPH-X variants is generated by combinatorial mutagenesis at the nucleic acid level and is encoded by a variegated gene library. A variegated library of EPH-X variants can be produced by, for example, enzymatically ligating a mixture of synthetic oligonucleotides into gene sequences such that a degenerate set of potential EPH-X sequences is expressible as individual polypeptides, or alternatively, as a set of larger fusion proteins (e.g., for phage display) containing the set of EPH-X sequences therein. There are a variety of methods which can be used to produce libraries of potential EPH-X variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential EPH-X sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the EPH-X protein coding sequences can be used to generate a variegated population of EPH-X fragments for screening and subsequent selection of variants of a EPH-X protein. In one embodiment, a library of coding sequence fragments can be generated by treating a double stranded PCR fragment of a EPH-X coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the EPH-X proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of EPH-X proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify EPH-X variants. See, e.g., Arkin and Yourvan, 1992. Proc. Natl. Acad. Sci. USA 89: 7811-7815; Delgrave, et al., 1993. Protein Engineering 6:327-331.

EPH-X Recombinant Expression Vectors and Host Cells

Another aspect of the invention pertains to vectors, preferably expression vectors, containing a nucleic acid encoding a EPH-X protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., EPH-X proteins, mutant forms of EPH-X proteins, fusion proteins, etc.).

The recombinant expression vectors of the invention can be designed for expression of EPH-X proteins in prokaryotic or eukaryotic cells. For example, EPH-X proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the EPH-X expression vector is a yeast expression vector. Examples of vectors for expression in yeast Saccharomyces cerivisae include pYepSec1 (Baldari, et al., 1987. EMBO J 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, EPH-X can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to EPH-X mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, EPH-X protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding EPH-X or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) EPH-X protein. Accordingly, the invention further provides methods for producing EPH-X protein using the host cells of the invention. In one embodiment, the method comprises culturing the host cell of invention (into which a recombinant expression vector encoding EPH-X protein has been introduced) in a suitable medium such that EPH-X protein is produced. In another embodiment, the method further comprises isolating EPH-X protein from the medium or the host cell.

Transgenic EPH-X Animals

The host cells of the invention can also be used to produce non-human transgenic animals. For example, in one embodiment, a host cell of the invention is a fertilized oocyte or an embryonic stem cell into which EPH-X protein-coding sequences have been introduced. Such host cells can then be used to create non-human transgenic animals in which exogenous EPH-X sequences have been introduced into their genome or homologous recombinant animals in which endogenous EPH-X sequences have been altered. Such animals are useful for studying the function and/or activity of EPH-X protein and for identifying and/or evaluating modulators of EPH-X protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous EPH-X gene has been altered by homologous recombination between the endogenous gene and an exogenous DNA molecule introduced into a cell of the animal, e.g., an embryonic cell of the animal, prior to development of the animal.

A transgenic animal of the invention can be created by introducing EPH-X-encoding nucleic acid into the male pronuclei of a fertilized oocyte (e.g., by microinjection, retroviral infection) and allowing the oocyte to develop in a pseudopregnant female foster animal. The human EPH-X cDNA sequences, i.e., any one of SEQ ID NO: 2n−1, wherein n is an integer between 1-46, can be introduced as a transgene into the genome of a non-human animal. Alternatively, a non-human homologue of the human EPH-X gene, such as a mouse EPH-X gene, can be isolated based on hybridization to the human EPH-X cDNA (described further supra) and used as a transgene. Intronic sequences and polyadenylation signals can also be included in the transgene to increase the efficiency of expression of the transgene. A tissue-specific regulatory sequence(s) can be operably-linked to the EPH-X transgene to direct expression of EPH-X protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the EPH-X transgene in its genome and/or expression of EPH-X mRNA in tissues or cells of the animals. A transgenic founder animal can then be used to breed additional animals carrying the transgene. Moreover, transgenic animals carrying a transgene-encoding EPH-X protein can further be bred to other transgenic animals carrying other transgenes.

To create a homologous recombinant animal, a vector is prepared which contains at least a portion of a EPH-X gene into which a deletion, addition or substitution has been introduced to thereby alter, e.g., functionally disrupt, the EPH-X gene. The EPH-X gene can be a human gene (e.g., the cDNA of any one of SEQ ID NO: 2n−1, wherein n is an integer between 1-46), but more preferably, is a non-human homologue of a human EPH-X gene. For example, a mouse homologue of human EPH-X gene of SEQ ID NO: 2n−1, wherein n is an integer between 1-46, can be used to construct a homologous recombination vector suitable for altering an endogenous EPH-X gene in the mouse genome. In one embodiment, the vector is designed such that, upon homologous recombination, the endogenous EPH-X gene is functionally disrupted (i.e., no longer encodes a functional protein; also referred to as a “knock out” vector).

Alternatively, the vector can be designed such that, upon homologous recombination, the endogenous EPH-X gene is mutated or otherwise altered but still encodes functional protein (e.g., the upstream regulatory region can be altered to thereby alter the expression of the endogenous EPH-X protein). In the homologous recombination vector, the altered portion of the EPH-X gene is flanked at its 5′- and 3′-termini by additional nucleic acid of the EPH-X gene to allow for homologous recombination to occur between the exogenous EPH-X gene carried by the vector and an endogenous EPH-X gene in an embryonic stem cell. The additional flanking EPH-X nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced EPH-X gene has homologously-recombined with the endogenous EPH-X gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The EPH-X nucleic acid molecules, EPH-X proteins, and anti-EPH-X antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a EPH-X protein or anti-EPH-X antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express EPH-X protein (e.g., via a recombinant expression vector in a host cell in gene therapy applications), to detect EPH-X mRNA (e.g., in a biological sample) or a genetic lesion in a EPH-X gene, and to modulate EPH-X activity, as described further, below. In addition, the EPH-X proteins can be used to screen drugs or compounds that modulate the EPH-X protein activity or expression as well as to treat disorders characterized by insufficient or excessive production of EPH-X protein or production of EPH-X protein forms that have decreased or aberrant activity compared to EPH-X wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-EPH-X antibodies of the invention can be used to detect and isolate EPH-X proteins and modulate EPH-X activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to EPH-X proteins or have a stimulatory or inhibitory effect on, e.g., EPH-X protein expression or EPH-X protein activity. The invention also includes compounds identified in the screening assays described herein.

In one embodiment, the invention provides assays for screening candidate or test compounds which bind to or modulate the activity of the membrane-bound form of a EPH-X protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S. Pat. No. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of EPH-X protein, or a biologically-active portion thereof, on the cell surface is contacted with a test compound and the ability of the test compound to bind to a EPH-X protein determined. The cell, for example, can of mammalian origin or a yeast cell. Determining the ability of the test compound to bind to the EPH-X protein can be accomplished, for example, by coupling the test compound with a radioisotope or enzymatic label such that binding of the test compound to the EPH-X protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of EPH-X protein, or a biologically-active portion thereof, on the cell surface with a known compound which binds EPH-X to form an assay mixture, contacting the assay mixture with a test compound, and determining the ability of the test compound to interact with a EPH-X protein, wherein determining the ability of the test compound to interact with a EPH-X protein comprises determining the ability of the test compound to preferentially bind to EPH-X protein or a biologically-active portion thereof as compared to the known compound.

In another embodiment, an assay is a cell-based assay comprising contacting a cell expressing a membrane-bound form of EPH-X protein, or a biologically-active portion thereof, on the cell surface with a test compound and determining the ability of the test compound to modulate (e.g., stimulate or inhibit) the activity of the EPH-X protein or biologically-active portion thereof. Determining the ability of the test compound to modulate the activity of EPH-X or a biologically-active portion thereof can be accomplished, for example, by determining the ability of the EPH-X protein to bind to or interact with a EPH-X target molecule. As used herein, a “target molecule” is a molecule with which a EPH-X protein binds or interacts in nature, for example, a molecule on the surface of a cell which expresses a EPH-X interacting protein, a molecule on the surface of a second cell, a molecule in the extracellular milieu, a molecule associated with the internal surface of a cell membrane or a cytoplasmic molecule. A EPH-X target molecule can be a non-EPH-X molecule or a EPH-X protein or polypeptide of the invention. In one embodiment, a EPH-X target molecule is a component of a signal transduction pathway that facilitates transduction of an extracellular signal (e.g. a signal generated by binding of a compound to a membrane-bound EPH-X molecule) through the cell membrane and into the cell. The target, for example, can be a second intercellular protein that has catalytic activity or a protein that facilitates the association of downstream signaling molecules with EPH-X.

Determining the ability of the EPH-X protein to bind to or interact with a EPH-X target molecule can be accomplished by one of the methods described above for determining direct binding. In one embodiment, determining the ability of the EPH-X protein to bind to or interact with a EPH-X target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a EPH-X-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a EPH-X protein or biologically-active portion thereof with a test compound and determining the ability of the test compound to bind to the EPH-X protein or biologically-active portion thereof. Binding of the test compound to the EPH-X protein can be determined either directly or indirectly as described above. In one such embodiment, the assay comprises contacting the EPH-X protein or biologically-active portion thereof with a known compound which binds EPH-X to form an assay mixture, contacting the assay mixture with a test compound, and determining the ability of the test compound to interact with a EPH-X protein, wherein determining the ability of the test compound to interact with a EPH-X protein comprises determining the ability of the test compound to preferentially bind to EPH-X or biologically-active portion thereof as compared to the known compound.

In still another embodiment, an assay is a cell-free assay comprising contacting EPH-X protein or biologically-active portion thereof with a test compound and determining the ability of the test compound to modulate (e.g. stimulate or inhibit) the activity of the EPH-X protein or biologically-active portion thereof. Determining the ability of the test compound to modulate the activity of EPH-X can be accomplished, for example, by determining the ability of the EPH-X protein to bind to a EPH-X target molecule by one of the methods described above for determining direct binding. In an alternative embodiment, determining the ability of the test compound to modulate the activity of EPH-X protein can be accomplished by determining the ability of the EPH-X protein further modulate a EPH-X target molecule. For example, the catalytic/enzymatic activity of the target molecule on an appropriate substrate can be determined as described, supra.

In yet another embodiment, the cell-free assay comprises contacting the EPH-X protein or biologically-active portion thereof with a known compound which binds EPH-X protein to form an assay mixture, contacting the assay mixture with a test compound, and determining the ability of the test compound to interact with a EPH-X protein, wherein determining the ability of the test compound to interact with a EPH-X protein comprises determining the ability of the EPH-X protein to preferentially bind to or modulate the activity of a EPH-X target molecule.

The cell-free assays of the invention are amenable to use of both the soluble form or the membrane-bound form of EPH-X protein. In the case of cell-free assays comprising the membrane-bound form of EPH-X protein, it may be desirable to utilize a solubilizing agent such that the membrane-bound form of EPH-X protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either EPH-X protein or its target molecule to facilitate separation of complexed from uncomplexed forms of one or both of the proteins, as well as to accommodate automation of the assay. Binding of a test compound to EPH-X protein, or interaction of EPH-X protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-EPH-X fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or EPH-X protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of EPH-X protein binding or activity determined using standard techniques.

Other techniques for immobilizing proteins on matrices can also be used in the screening assays of the invention. For example, either the EPH-X protein or its target molecule can be immobilized utilizing conjugation of biotin and streptavidin. Biotinylated EPH-X protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with EPH-X protein or target molecules, but which do not interfere with binding of the EPH-X protein to its target molecule, can be derivatized to the wells of the plate, and unbound target or EPH-X protein trapped in the wells by antibody conjugation. Methods for detecting such complexes, in addition to those described above for the GST-immobilized complexes, include immunodetection of complexes using antibodies reactive with the EPH-X protein or target molecule, as well as enzyme-linked assays that rely on detecting an enzymatic activity associated with the EPH-X protein or target molecule.

In another embodiment, modulators of EPH-X protein expression are identified in a method wherein a cell is contacted with a candidate compound and the expression of EPH-X mRNA or protein in the cell is determined. The level of expression of EPH-X mRNA or protein in the presence of the candidate compound is compared to the level of expression of EPH-X mRNA or protein in the absence of the candidate compound. The candidate compound can then be identified as a modulator of EPH-X mRNA or protein expression based upon this comparison. For example, when expression of EPH-X mRNA or protein is greater (i.e., statistically significantly greater) in the presence of the candidate compound than in its absence, the candidate compound is identified as a stimulator of EPH-X mRNA or protein expression. Alternatively, when expression of EPH-X mRNA or protein is less (statistically significantly less) in the presence of the candidate compound than in its absence, the candidate compound is identified as an inhibitor of EPH-X mRNA or protein expression. The level of EPH-X mRNA or protein expression in the cells can be determined by methods described herein for detecting EPH-X mRNA or protein.

In yet another aspect of the invention, the EPH-X proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with EPH-X (“EPH-X-binding proteins” or “EPH-X-bp”) and modulate EPH-X activity. Such EPH-X-binding proteins are also involved in the propagation of signals by the EPH-X proteins as, for example, upstream or downstream elements of the EPH-X pathway.

The two-hybrid system is based on the modular nature of most transcription factors, which consist of separable DNA-binding and activation domains. Briefly, the assay utilizes two different DNA constructs. In one construct, the gene that codes for EPH-X is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a EPH-X-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with EPH-X.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the EPH-X sequences of SEQ ID NO: 2n−1, wherein n is an integer between 1-46, or fragments or derivatives thereof, can be used to map the location of the EPH-X genes, respectively, on a chromosome. The mapping of the EPH-X sequences to chromosomes is an important first step in correlating these sequences with genes associated with disease.

Briefly, EPH-X genes can be mapped to chromosomes by preparing PCR primers (preferably 15-25 bp in length) from the EPH-X sequences. Computer analysis of the EPH-X, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the EPH-X sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the EPH-X sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the EPH-X gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The EPH-X sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the EPH-X sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The EPH-X sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO: 2n−1, wherein n is an integer between 1-46, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining EPH-X protein and/or nucleic acid expression as well as EPH-X activity, in the context of a biological sample (e.g., blood, serum, cells, tissue) to thereby determine whether an individual is afflicted with a disease or disorder, or is at risk of developing a disorder, associated with aberrant EPH-X expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with EPH-X protein, nucleic acid expression or activity. For example, mutations in a EPH-X gene can be assayed in a biological sample. Such assays can be used for prognostic or predictive purpose to thereby prophylactically treat an individual prior to the onset of a disorder characterized by or associated with EPH-X protein, nucleic acid expression, or biological activity.

Another aspect of the invention provides methods for determining EPH-X protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of EPH-X in clinical trials.

These and other agents are described in further detail in the following sections.

Diagnostic Assays

An exemplary method for detecting the presence or absence of EPH-X in a biological sample involves obtaining a biological sample from a test subject and contacting the biological sample with a compound or an agent capable of detecting EPH-X protein or nucleic acid (e.g., mRNA, genomic DNA) that encodes EPH-X protein such that the presence of EPH-X is detected in the biological sample. An agent for detecting EPH-X mRNA or genomic DNA is a labeled nucleic acid probe capable of hybridizing to EPH-X mRNA or genomic DNA. The nucleic acid probe can be, for example, a full-length EPH-X nucleic acid, such as the nucleic acid of SEQ ID NO: 2n−1, wherein n is an integer between 1-46, or a portion thereof, such as an oligonucleotide of at least 15, 30, 50, 100, 250 or 500 nucleotides in length and sufficient to specifically hybridize under stringent conditions to EPH-X mRNA or genomic DNA. Other suitable probes for use in the diagnostic assays of the invention are described herein.

An agent for detecting EPH-X protein is an antibody capable of binding to EPH-X protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect EPH-X mRNA, protein, or genomic DNA in a biological sample in vitro as well as in vivo. For example, in vitro techniques for detection of EPH-X mRNA include Northern hybridizations and in situ hybridizations. In vitro techniques for detection of EPH-X protein include enzyme linked immunosorbent assays (ELISAs), Western blots, immunoprecipitations, and immunofluorescence. In vitro techniques for detection of EPH-X genomic DNA include Southern hybridizations. Furthermore, in vivo techniques for detection of EPH-X protein include introducing into a subject a labeled anti-EPH-X antibody. For example, the antibody can be labeled with a radioactive marker whose presence and location in a subject can be detected by standard imaging techniques.

In one embodiment, the biological sample contains protein molecules from the test subject. Alternatively, the biological sample can-contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting EPH-X protein, mRNA, or genomic DNA, such that the presence of EPH-X protein, mRNA or genomic DNA is detected in the biological sample, and comparing the presence of EPH-X protein, mRNA or genomic DNA in the control sample with the presence of EPH-X protein, mRNA or genomic DNA in the test sample.

The invention also encompasses kits for detecting the presence of EPH-X in a biological sample. For example, the kit can comprise: a labeled compound or agent capable of detecting EPH-X protein or mRNA in a biological sample; means for determining the amount of EPH-X in the sample; and means for comparing the amount of EPH-X in the sample with a standard. The compound or agent can be packaged in a suitable container. The kit can further comprise instructions for using the kit to detect EPH-X protein or nucleic acid.

Prognostic Assays

The diagnostic methods described herein can furthermore be utilized to identify subjects having or at risk of developing a disease or disorder associated with aberrant EPH-X expression or activity. For example, the assays described herein, such as the preceding diagnostic assays or the following assays, can be utilized to identify a subject having or at risk of developing a disorder associated with EPH-X protein, nucleic acid expression or activity. Alternatively, the prognostic assays can be utilized to identify a subject having or at risk for developing a disease or disorder. Thus, the invention provides a method for identifying a disease or disorder associated with aberrant EPH-X expression or activity in which a test sample is obtained from a subject and EPH-X protein or nucleic acid (e.g., mRNA, genomic DNA) is detected, wherein the presence of EPH-X protein or nucleic acid is diagnostic for a subject having or at risk of developing a disease or disorder associated with aberrant EPH-X expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant EPH-X expression or activity. For example, such methods can be used to determine whether a subject can be effectively treated with an agent for a disorder. Thus, the invention provides methods for determining whether a subject can be effectively treated with an agent for a disorder associated with aberrant EPH-X expression or activity in which a test sample is obtained and EPH-X protein or nucleic acid is detected (e.g., wherein the presence of EPH-X protein or nucleic acid is diagnostic for a subject that can be administered the agent to treat a disorder associated with aberrant EPH-X expression or activity).

The methods of the invention can also be used to detect genetic lesions in a EPH-X gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a EPH-X-protein, or the misexpression of the EPH-X gene. For example, such genetic lesions can be detected by ascertaining the existence of at least one of: (i) a deletion of one or more nucleotides from a EPH-X gene; (ii) an addition of one or more nucleotides to a EPH-X gene; (iii) a substitution of one or more nucleotides of a EPH-X gene, (iv) a chromosomal rearrangement of a EPH-X gene; (v) an alteration in the level of a messenger RNA transcript of a EPH-X gene, (vi) aberrant modification of a EPH-X gene, such as of the methylation pattern of the genomic DNA, (vii) the presence of a non-wild-type splicing pattern of a messenger RNA transcript of a EPH-X gene, (viii) a non-wild-type level of a EPH-X protein, (ix) allelic loss of a EPH-X gene, and (x) inappropriate post-translational modification of a EPH-X protein. As described herein, there are a large number of assay techniques known in the art which can be used for detecting lesions in a EPH-X gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the EPH-X-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a EPH-X gene under conditions such that hybridization and amplification of the EPH-X gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a EPH-X gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in EPH-X can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in EPH-X can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the EPH-X gene and detect mutations by comparing the sequence of the sample EPH-X with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the EPH-X gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type EPH-X sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in EPH-X cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a EPH-X sequence, e.g., a wild-type EPH-X sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in EPH-X genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control EPH-X nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a EPH-X gene.

Furthermore, any cell type or tissue, preferably peripheral blood leukocytes, in which EPH-X is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on EPH-X activity (e.g., EPH-X gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of EPH-X protein, expression of EPH-X nucleic acid, or mutation content of EPH-X genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of EPH-X protein, expression of EPH-X nucleic acid, or mutation content of EPH-X genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a EPH-X modulator, such as a modulator identified by one of the exemplary screening assays described herein.

Monitoring of Effects During Clinical Trials

Monitoring the influence of agents (e.g., drugs, compounds) on the expression or activity of EPH-X (e.g., the ability to modulate aberrant cell proliferation and/or differentiation) can be applied not only in basic drug screening, but also in clinical trials. For example, the effectiveness of an agent determined by a screening assay as described herein to increase EPH-X gene expression, protein levels, or upregulate EPH-X activity, can be monitored in clinical trails of subjects exhibiting decreased EPH-X gene expression, protein levels, or downregulated EPH-X activity. Alternatively, the effectiveness of an agent determined by a screening assay to decrease EPH-X gene expression, protein levels, or downregulate EPH-X activity, can be monitored in clinical trails of subjects exhibiting increased EPH-X gene expression, protein levels, or upregulated EPH-X activity. In such clinical trials, the expression or activity of EPH-X and, preferably, other genes that have been implicated in, for example, a cellular proliferation or immune disorder can be used as a “read out” or markers of the immune responsiveness of a particular cell.

By way of example, and not of limitation, genes, including EPH-X, that are modulated in cells by treatment with an agent (e.g., compound, drug or small molecule) that modulates EPH-X activity (e.g., identified in a screening assay as described herein) can be identified. Thus, to study the effect of agents on cellular proliferation disorders, for example, in a clinical trial, cells can be isolated and RNA prepared and analyzed for the levels of expression of EPH-X and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of EPH-X or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a EPH-X protein, mRNA, or genomic DNA in the preadministration sample; (iii) obtaining one or more post-administration samples from the subject; (iv) detecting the level of expression or activity of the EPH-X protein, mRNA, or genomic DNA in the post-administration samples; (v) comparing the level of expression or activity of the EPH-X protein, mRNA, or genomic DNA in the pre-administration sample with the EPH-X protein, mRNA, or genomic DNA in the post administration sample or samples; and (vi) altering the administration of the agent to the subject accordingly. For example, increased administration of the agent may be desirable to increase the expression or activity of EPH-X to higher levels than detected, i.e., to increase the effectiveness of the agent. Alternatively, decreased administration of the agent may be desirable to decrease expression or activity of EPH-X to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant EPH-X expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant EPH-X expression or activity, by administering to the subject an agent that modulates EPH-X expression or at least one EPH-X activity. Subjects at risk for a disease that is caused or contributed to by aberrant EPH-X expression or activity can be identified by, for example, any or a combination of diagnostic or prognostic assays as described herein. Administration of a prophylactic agent can occur prior to the manifestation of symptoms characteristic of the EPH-X aberrancy, such that a disease or disorder is prevented or, alternatively, delayed in its progression. Depending upon the type of EPH-X aberrancy, for example, a EPH-X agonist or EPH-X antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating EPH-X expression or activity for therapeutic purposes. The modulatory method of the invention involves contacting a cell with an agent that modulates one or more of the activities of EPH-X protein activity associated with the cell. An agent that modulates EPH-X protein activity can be an agent as described herein, such as a nucleic acid or a protein, a naturally-occurring cognate ligand of a EPH-X protein, a peptide, a EPH-X peptidomimetic, or other small molecule. In one embodiment, the agent stimulates one or more EPH-X protein activity. Examples of such stimulatory agents include active EPH-X protein and a nucleic acid molecule encoding EPH-X that has been introduced into the cell. In another embodiment, the agent inhibits one or more EPH-X protein activity. Examples of such inhibitory agents include antisense EPH-X nucleic acid molecules and anti-EPH-X antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a EPH-X protein or nucleic acid molecule. In one embodiment, the method involves administering an agent (e.g., an agent identified by a screening assay described herein), or combination of agents that modulates (e.g., up-regulates or down-regulates) EPH-X expression or activity. In another embodiment, the method involves administering a EPH-X protein or nucleic acid molecule as therapy to compensate for reduced or aberrant EPH-X expression or activity.

Stimulation of EPH-X activity is desirable in situations in which EPH-X is abnormally downregulated and/or in which increased EPH-X activity has a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The EPH-X nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the EPH-X protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the EPH-X protein, and the EPH-X protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

### EXAMPLE 2

Molecular Cloning of CG54020-02

Materials and Methods

The predicted open reading frame of cgAL035703 (also known as CG54020-01) encodes a novel Type I membrane protein with a transmembrane domain between amino acid residues 540-566 (predicted by PSORT). SIGNALP predicted a signal peptidase cleavage site between residues 27 and 28. The mature form of the extracellular domain of CG54020-01, containing amino acid residues 28 to 538, was targeted for cloning. Oligonucleotide primers were designed to PCR amplify the sequence encoding the mature extracellular domain of cgAL035703. The forward primer included an in-frame BamHI site and the reverse primer contained an in-frame XhoI restriction site for cloning purposes.

PCR reactions contained 5 ng human hypothalamus cDNA template, 1 μM of each of the AL035703 forward and reverse primers, 5 μmoles dNTP (Clontech Laboratories, Palo Alto Calif.) and 1 μL of 50× Advantage-HF 2 polymerase (Clontech) in 50 μL volume. The following reaction conditions were used:

| a) | 96° C. | 3 minutes |
|---|---|---|
| b) | 96° C. | 30 seconds denaturation |
| c) | 70° C. | 30 seconds, primer annealing. This temperature |
| | | was gradually decreased by 1° C./cycle |
| d) | 72° C. | 3 minutes extension. |
| Repeat steps b-d 10 times | | |
| e) | 96° C. | 30 seconds denaturation |
| f) | 60° C. | 30 seconds annealing |
| g) | 72° C. | 3 minutes extension |
| Repeat steps e-g 25 times | | |
| h) | 72° C. | 5 minutes final extension |

A single, 1500 bp amplified product was detected by agarose gel electrophoresis. The product was isolated and ligated into the pCR2.1 vector (Invitrogen Corp, Carlsbad Calif.).

The construct was sequenced using the following gene-specific primers:

Results

The cloned insert was verified as an open reading frame encoding amino acids 28 to 538 of the CG54020-01 (SEQ ID NO: 2) protein. This construct is called pCR2.1-cgAL035703-S340-1C and is also known as CG54020-02 (SEQ ID NO: 4).

### EXAMPLE 3

Transient Expression of CG54020-02 in Human Embryonic Kidney 293 Cells

Materials and Methods

A 1.5 kB BamHI-XhoI fragment from pCR2.1-cgAL035703-S340-1C, containing the CG54020-02 sequence, was subcloned into the BamHI-XhoI digested mammalian expression vector pCEP4/Sec (CuraGen Corporation). The pCEP4Sec vector expresses the protein of interest with an in-frame iGk secretion signal at the N terminus and a V5/His₆ tag at the C terminus. The pCEP4Sec/CG54020-02 construct was transiently transfected into HEK293 cells using the LipofectaminePlus reagent following the manufacturer's instructions (Gibco/BRL, Gaithesburg, Md.). HEK293 cells were grown in DMEM supplemented with 10% FBS, 2 mM glutamine and pen-strep. The cell pellet and supernatant were harvested 72 h post transfection and examined for CG54020-02 expression by Western blot (reducing conditions) using an anti-V5 antibody.

Results

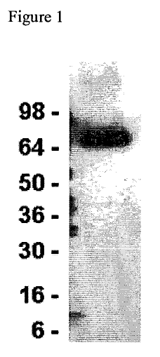

An approximately 65 kDa protein was detected in the conditioned media, indicating that the molecule is secreted (FIG. 1). The conditioned media was submitted to metal affinity based protein purification.

### EXAMPLE 4

Stable Expression of CG54020-02 in CHO-K1 Cells

Materials and Methods

The insert from pCR2.1-cgAL035703-S340-1C (Example 3) was subcloned into the pEE14.4Sec mammalian expression vector (CuraGen Corporation). The vector carries the glutamine synthase selective marker that allows the selection of stable clones in the presence of methionine sulfoximine (MSX). The final MSX concentration was 100 μM for selection and the culture was maintained in the presence of 25 μM MSX. The pEE14.4Sec/CG54020-02 plasmid was transfected into CHO-K1 cells and stable clones were established. EXCel1302 media (JRH Biotech, City, State) was supplemented with 5% FBS, nucleosides and nonessential amino acids (GS supplement, HT supplement; JRH Biotech, City, State).

Results

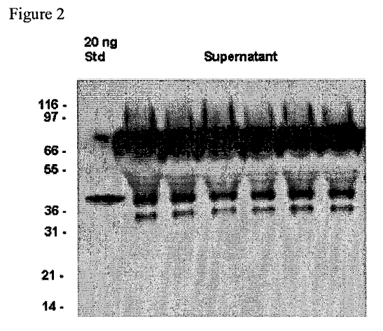

The conditioned media from 12 stable clones was analyzed by Western analysis using the anti-V5 antibody. Analysis of CG54020-02 expression from representative clones is shown in FIG. 2. An approximately 65 kDa molecule was detected in the supernatant indicating that the CG54020-02 protein is secreted.

### EXAMPLE 5

Identification of Human Ephrin A Receptor Gene Variants and SNPs

Materials and Methods

SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen Corporation's SeqCalling technology that is disclosed in full in U.S. Ser. Nos. 09/417,386 filed Oct. 13, 1999, and 09/614,505 filed Jul. 11, 2000. Sequence traces were evaluated manually and edited for corrections if appropriate. cDNA sequences from all samples were assembled together, sometimes including public human sequences, using bioinformatics programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

Method of novel SNP Identification: SNPs were identified by analyzing sequence assemblies using CuraGen's proprietary SNPTool algorithm. SNPTool identifies variation in assemblies with the following criteria: SNPs are not analyzed within 10 base pairs on both ends of an alignment; window size (number of bases in a view) is 10; the allowed number of mismatches in a window is 2; minimum SNP base quality (PHRED score) is 23; and the minimum number of changes to score a SNP is two per assembly position. SNPTool analyzes the assembly and displays SNP positions, associated individual variant sequences in the assembly, the depth of the assembly at that given position, the putative assembly allele frequency, and the SNP sequence variation. Sequence traces were then selected and brought into view for manual validation. The consensus assembly sequence was imported into CuraTools along with variant sequence changes to identify potential amino acid changes resulting from the SNP sequence variation. Comprehensive SNP data analysis was then exported into the SNPCalling database. Variants are reported individually but any combination of all or a select subset of variants is also included as contemplated EPH-X embodiments of the invention.

Molecular Cloning of CG54020-04 and -05 by Exon Linking: The cDNA coding for the CG54020-04 and CG54020-05 sequences were cloned by the polymerase chain reaction (PCR) using the primers: 5′-GTGCGGAGAGCGAGGGAG-3′ (SEQ ID NO: 40) and 5′-CATGACCTGGGGTGGGCTT-3′ (SEQ ID NO: 41). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

The PCR product derived by exon linking, covering the entire open reading frame, was cloned into the pCR2.1 vector from Invitrogen to provide the following clones:

138920::Hs_S1973309.1043228.09 (CG54020-04)

138920::Hs_S1973309.1043228.011 (CG54020-05)

Results

SNPs Identified in CG54020-01 Gene: Eight polymorphic variants of CG54020-01 were identified and are shown in Table 2.

TABLE 2
SNPs identified in CG54020-01 nucleotide sequence

| | Nucleotides | | | Amino Acids | | |
|---|---|---|---|---|---|---|
| Variant | Position | Initial | Modified | Position | Initial | Modified |
| 13382340 | 222 | C | T | 74 | Asn | Asn |
| 13375084 | 899 | C | T | 300 | Ala | Val |
| 13375087 | 1250 | G | A | 417 | Gly | Asp |
| 13375086 | 1253 | T | C | 418 | Val | Ala |
| 13375085 | 1271 | A | G | 424 | Glu | Gly |
| 13375088 | 1724 | A | G | 575 | Gln | Arg |
| 13375089 | 2614 | C | T | 872 | Leu | Phe |
| 13375090 | 2800 | A | G | 934 | Thr | Ala |

### EXAMPLE 6

Quantitative Expression Analysis of CG54020 in Various cells and Tissues

Materials and Methods

RTQ-PCR Technology: The quantitative expression of CG54020 was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISMS 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s: 18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 μl or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 59° C. primer optimal Tm; 2° C. maximum primer difference (if probe does not have 5′ G, probe Tₘ must be 10° C. greater than primer Tₘ; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using Taqman® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 95° C. for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1× TaqMan® Universal Master mix (Applied Biosystems; catalog No. 4324020), following the manufacturer's instructions. PCR amplification and analysis were done as described above.

Panels 1, 1.1, 1.2, and 1.3D: Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (p1. eff or pl effusion) and * indicates established from metastasis.

General_screening_panel_v1.4, v1.5, v1.6 and v1.7: Panels 1.4, 1.5, 1.6 and 1.7 were as described for Panels 1, 1.1, 1.2 and 1.3D, above except that normal tissue samples were pooled from 2 to 5 different adults or fetuses.

ARDAIS Panel v1.0 and v1.1: The ARDAIS panels v1.0 and v1.1 included 2 controls and 22 test samples including: human lung adenocarcinomas, lung squamous cell carcinomas (SCC), and in some cases matched adjacent normal tissues (NAT) obtained from Ardais (Lexington, Mass.). Unmatched malignant and non-malignant RNA samples from lungs with gross histopathological assessment of tumor differentiation grade and stage (SI, stage I; SII, stage II; SIII, stage III) and clinical state of the patient were obtained from Ardais.

ARDAIS Breast v1.0: ARDAIS Breast v1.0 panel included 2 controls and 71 test samples of human breast malignancies and in some cases matched adjacent normal tissues (NAT) obtained from Ardais (Lexington, Mass.). RNA from unmatched malignant and non-malignant breast samples with gross histopathological assessment of tumor differentiation grade and stage and clinical state of the patient were also obtained from Ardais.

Panels 3D, 3.1 and 3.2: Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

Results:

Expression of gene CG54020 was assessed using the primer-probe set Ag7884, described in Table 3A. Ag7884 recognizes all variants of CG54020 disclosed in this application (CG54020-01 to -05). Results of the RTQ-PCR runs are shown in Tables 3B, 3C, 3D and 3E.

TABLE 3A

Probe Name Ag7884

| | | | | |
|---|---|---|---|---|
| | | | Start | |
| Primers | Sequences | Length | Position | SEQ ID No |

| | | | | |
|---|---|---|---|---|
| Forward | 5′-gcacacaagaaagccagttcct-3′ | 22 | 407 | 37 |
| Probe | TET-5′-aaaatcgacaccattgcggccga-3′-TAMRA | 23 | 430 | 38 |
| Reverse | 5′-ggtcggcacctgtgaagct-3′ | 19 | 457 | 39 |

TABLE 3B
Ardais Breast1.0
Column A - Rel. Exp.(%) Ag7884, Run 394516267

| Tissue Name | A | Tissue Name | A |
|---|---|---|---|
| 97739_Breast cancer | 3.7 | 97764_Breast cancer | 0.3 |
| (CHTN20676) | | node metastasis (OD06083) | |
| 105689_Breast cancer | 0.0 | 108847_5A Breast Cancer | 0.3 |
| 2A | | | |
| 111297_Metastatic | 1.0 | 116421_Breast cancer (6314) | 0.1 |
| Breast cancer (9369)* | | | |
| 145848_Breast cancer | 0.8 | 145854_Breast cancer (9B8) | 3.3 |
| (9B6) | | | |
| 145859_Breast cancer | 0.1 | 153627_Breast cancer (D34) | 0.3 |
| (9EC) | | | |
| 153632_Breast cancer | 0.9 | 153636_Breast cancer (D3D) | 0.5 |
| (D39) | | | |
| 153643_Breast cancer | 0.3 | 164668_Breast cancer (6314) | 48.6 |
| (D44) | | | |
| 164672_Breast cancer | 2.6 | 164677_Breast cancer (5272) | 0.5 |
| (7464) | | | |
| 164681_Breast cancer | 9.8 | 164685_Breast cancer (0170) | 11.8 |
| (5787) | | | |
| 97748_Breast cancer | 0.4 | 98857_Breast cancer | 2.8 |
| (CHTN20931) | | (OD06397-12) | |
| 105690_Breast NAT | 0.0 | 111288_Breast NAT (3367) | 1.4 |
| 2B | | | |
| 111302_Breast NAT | 13.4 | 116424_Breast cancer (3388)* | 0.0 |
| (6314) | | | |
| 145850_Breast cancer | 0.6 | 153628_Breast cancer (D35) | 1.4 |
| (9C7) | | | |
| 149844_Breast cancer | 0.0 | 153637_Breast cancer (D3E) | 4.4 |
| (24178) | | | |
| 153633_Breast cancer | 1.2 | 164669_Breast cancer (6992) | 0.2 |
| (D3A) | | | |
| 153644_Breast cancer | 21.5 | 164678_Breast cancer (5297) | 0.1 |
| (D45) | | | |
| 164673_Breast cancer | 32.1 | 164686_Breast cancer (0732) | 0.0 |
| (8452) | | | |
| 164682_Breast cancer | 2.0 | 105687_Breast cancer (1A) | 0.0 |
| (6342)* | | | |
| 97751_Breast cancer | 0.1 | 111289_Breast cancer (3369)* | 0.2 |
| (CHTN21053) | | | |
| 105694_Breast NAT | 0.3 | 116425_Breast NAT (3388) | 0.0 |
| 5B | | | |
| 116417_Breast cancer | 0.0 | 145857_Breast cancer (9F0) | 0.9 |
| (3367)* | | | |
| 145852_Breast cancer | 1.6 | 153630_Breast cancer (D37) | 98.6 |
| (A1A) | | | |
| 151097_Breast cancer | 0.8 | 153638_Breast cancer (D3F) | 2.6 |
| (CHTN24298) | | | |
| 153634_Breast cancer | 0.3 | 164670_Breast cancer (7078) | 4.7 |
| (D3B) | | | |
| 155797_Breast cancer | 1.7 | 164679_Breast cancer (5486) | 0.1 |
| (EA6) | | | |
| 164674_Breast cancer | 4.0 | 164687_Breast cancer (5881) | 7.4 |
| (8811) | | | |
| 164683_Breast cancer | 0.0 | 105688_Breast NAT (1B) | 0.0 |
| (6470) | | | |
| 97763_Breast cancer | 0.0 | 111290_Breast NAT (3369)* | 0.0 |
| (OD06083) | | | |
| 108830_Breast cancer | 0.1 | 145846_Breast cancer (9B7) | 36.9 |
| metastasis (OD06855)* | | | |
| 116418_Breast cancer | 0.4 | 145858_Breast cancer (9B4) | 1.6 |
| (3378)* | | | |
| 145853_Breast cancer | 1.6 | 153631_Breast cancer (D38) | 0.6 |
| (9F3) | | | |
| 153432_Breast cancer | 37.6 | 153639_Breast cancer (D40) | 100.0 |
| (CHTN 24652) | | | |
| 153635_Breast cancer | 1.5 | 164671_Breast cancer (7082) | 0.4 |
| (D3C) | | | |
| 164667_Breast cancer | 0.6 | 164680_Breast cancer (5705) | 37.6 |
| (5785) | | | |
| 164676_Breast cancer | 3.4 | 164688_Breast cancer (7222) | 0.0 |
| (5070) | | | |
| 164684_Breast cancer | 1.1 | | |
| (6509) | | | |

TABLE 3C
Ardais Panel 1.1
Column A - Rel. Exp.(%) Ag7884, Run 315065605

| Tissue Name | A | Tissue Name | A |
|---|---|---|---|
| Lung adenocarcinoma SI A | 100.0 | Lung SCC SI A | 0.9 |
| Lung adenocarcinoma SI B | 31.9 | Lung SCC SI B NAT | 0.1 |
| Lung adenocarcinoma SI B | 0.2 | Lung SCC SI C | 0.5 |
| NAT | | | |
| Lung adenocarcinoma SI C | 0.2 | Lung SCC SI C NAT | 1.5 |
| Lung adenocarcinoma SI C | 0.2 | Lung SCC SI D | 0.4 |
| NAT | | | |
| Lung adenocarcinoma SII A | 55.9 | Lung SCC SI D NAT | 0.3 |
| Lung adenocarcinoma SII A | 0.1 | Lung SCC SII A | 11.5 |
| NAT | | | |
| Lung adenocarcinoma SII C | 0.2 | Lung SCC SII B | 0.7 |
| NAT | | | |
| Lung adenocarcinoma SIII A | 1.4 | Lung SCC SIII A | 1.2 |
| Lung adenocarcinoma SIII B | 0.3 | Lung SCC SIII A NAT | 0.2 |
| Lung adenocarcinoma SIII C | 1.6 | | |

TABLE 3D
General_screening_panel_v1.7
Column A - Rel. Exp.(%) Ag7884, Run 318008718

| Tissue Name | A | Tissue Name | A |
|---|---|---|---|
| Adipose | 0.0 | Gastric ca. (liver met.) | 0.0 |
| | | NCI-N87 | |
| HUVEC | 0.0 | Stomach | 0.0 |
| Melanoma* Hs688(A).T | 0.0 | Colon ca. SW-948 | 0.0 |
| Melanoma* Hs688(B).T | 0.1 | Colon ca. SW480 | 0.0 |
| Melanoma (met) | 0.6 | Colon ca. (SW480 met) SW620 | 7.7 |
| SK-MEL-5 | | | |
| Testis | 2.3 | Colon ca. HT29 | 0.0 |
| Prostate ca. | 0.0 | Colon ca. HCT-116 | 0.1 |
| (bone met) PC-3 | | | |
| Prostate ca. DU145 | 0.1 | Colon cancer tissue | 0.0 |
| Prostate pool | 0.0 | Colon ca. SW1116 | 0.1 |
| Uterus pool | 0.0 | Colon ca. Colo-205 | 1.5 |
| Ovarian ca. OVCAR-3 | 0.0 | Colon ca. SW-48 | 0.0 |
| Ovarian ca. (ascites) | 0.0 | Colon | 0.0 |
| SK-OV-3 | | | |
| Ovarian ca. OVCAR-4 | 1.4 | Small Intestine | 0.0 |
| Ovarian ca. OVCAR-5 | 0.0 | Fetal Heart | 0.0 |
| Ovarian ca. IGROV-1 | 1.7 | Heart | 0.0 |
| Ovarian ca. OVCAR-8 | 0.0 | Lymph Node pool 1 | 0.0 |
| Ovary | 0.0 | Lymph Node pool 2 | 0.1 |
| Breast ca. MCF-7 | 1.2 | Fetal Skeletal Muscle | 0.0 |
| Breast ca. | 0.0 | Skeletal Muscle pool | 0.0 |
| MDA-MB-231 | | | |
| Breast ca. BT-549 | 0.0 | Skeletal Muscle | 0.0 |
| Breast ca. T47D | 2.9 | Spleen | 3.2 |
| Breast pool | 0.0 | Thymus | 0.0 |
| Trachea | 0.1 | CNS cancer (glio/astro) SF-268 | 0.0 |
| Lung | 0.0 | CNS cancer (glio/astro) T98G | 0.0 |
| Fetal Lung | 0.1 | CNS cancer (neuro;met) | 0.0 |
| | | SK-N-AS | |
| Lung ca. NCI-N417 | 1.7 | CNS cancer (astro) SF-539 | 0.0 |
| Lung ca. LX-1 | 0.0 | CNS cancer (astro) SNB-75 | 0.2 |
| Lung ca. NCI-H146 | N/A | CNS cancer (glio) SNB-19 | 0.0 |
| Lung ca. SHP-77 | 100.0 | CNS cancer (glio) SF-295 | 0.0 |
| Lung ca. NCI-H23 | 1.4 | Brain (Amygdala) | 2.1 |
| Lung ca. NCI-H460 | 11.5 | Brain (Cerebellum) | 4.7 |
| Lung ca. HOP-62 | 0.0 | Brain (Fetal) | 10.7 |
| Lung ca. NCI-H522 | 3.1 | Brain (Hippocampus) | 2.8 |
| Lung ca. DMS-114 | 3.9 | Cerebral Cortex pool | 0.8 |
| Liver | 0.0 | Brain (Substantia nigra) | 0.8 |
| Fetal Liver | 0.1 | Brain (Thalamus) | 2.8 |
| Kidney pool | 0.0 | Brain (Whole) | 7.5 |
| Fetal Kidney | 0.3 | Spinal Cord | 0.7 |
| Renal ca. 786-0 | 0.2 | Adrenal Gland | 8.1 |
| Renal ca. A498 | 0.0 | Pituitary Gland | 1.0 |
| Renal ca. ACHN | 0.0 | Salivary Gland | 0.0 |
| Renal ca. UO-31 | 0.0 | Thyroid | 0.2 |
| Renal ca. TK-l0 | 0.0 | Pancreatic ca. PANC-1 | 0.0 |
| Bladder | 0.0 | Pancreas pool | 0.8 |

TABLE 3E
Oncology_cell_line_screening_panel_v3.2
Column A - Rel. Exp.(%) Ag7884, Run 315065045

| Tissue Name | A | Tissue Name | A |
|---|---|---|---|
| 94905_Daoy_Medullo- | 0.2 | 94954_Ca Ski_Cervical | 0.0 |
| blastoma/Cerebellum | | epidermoid carcinoma | |
| | | (metastasis) | |
| 94906_TE671_Medullo- | 1.9 | 94955_ES-2_Ovarian | 0.0 |
| blastom/Cerebellum | | clear cell carcinoma | |
| 94907_D283 | 55.5 | 94957_Ramos/6h | 0.0 |
| Med_Medulloblastoma/ | | stim_Stimulated | |
| Cerebellum | | with PMA/ionomycin 6h | |
| 94908_PFSK-1_Primitive | 0.0 | 94958_Ramos/14h | 0.0 |
| Neuroectodermal/Cerebellum | | stim_Stimulated | |
| | | with PMA/ionomycin 14h | |
| 94909_XF-498_CNS | 0.0 | 94962_MEG-01_Chronic | 24.7 |
| | | myelogenous | |
| | | leukemia (megokaryoblast) | |
| 94910_SNB-78_CNS/glioma | 0.0 | 94963_Raji_Burkitt's | 0.0 |
| | | lymphoma | |
| 94911_SF-268_CNS/ | 0.0 | 94964_Daudi_Burkitt's | 0.0 |
| glioblastoma | | lymphoma | |
| 94912_T98G_Glioblastoma | 0.3 | 94965_U266_B-cell | 0.0 |
| | | plasmacytoma/myeloma | |
| 96776_SK-N-SH_Neuro- | 1.1 | 94968_CA46_Burkitt's | 0.0 |
| blastoma (metastasis) | | lymphoma | |
| 94913_SF-295_CNS/ | 0.7 | 94970_RL_non-Hodgkin's | 0.0 |
| glioblastoma | | B-cell lymphoma | |
| 132565_NT2 pool | 10.2 | 94972_JM1_pre-B-cell | 0.0 |
| | | lymphoma/leukemia | |
| 94914_Cerebellum | 6.2 | 94973_Jurkat_T cell | 0.0 |
| | | leukemia | |
| 96777_Cerebellum | 8.1 | 94974_TF-1_Erythro- | 0.8 |
| | | leukemia | |
| 94916_NCI-H292_Muco- | 0.0 | 94975_HUT 78_T-cell | 0.0 |
| epidermoid lung carcinoma | | lymphoma | |
| 94917_DMS-114_Small | 6.9 | 94977_U937_Histiocytic | 0.0 |
| cell lung cancer | | lymphoma | |
| 94918_DMS-79_Small | 12.5 | 94980_KU-812_Myelo- | 0.2 |
| cell lung cancer/ | | genous leukemia | |
| neuroendocrine | | | |
| 94919_NCI-H146_Small | 13.6 | 94981_769-P_Clear cell | 0.0 |
| cell lung cancer/ | | renal carcinoma | |
| neuroendocrine | | | |
| 94920_NCI-H526_Small | 23.3 | 94983_Caki-2_Clear cell | 0.2 |
| cell lung cancer/ | | renal carcinoma | |
| neuroendocrine | | | |
| 94921_NCI-N417_Small | 6.9 | 94984_SW 839_Clear | 0.0 |
| cell lung cancer/ | | cell renal carcinoma | |
| neuroendocrine | | | |
| 94923_NCI-H82_Small | 72.7 | 94986_G401_Wilms' | 0.2 |
| cell lung cancer/ | | tumor | |
| neuroendocrine | | | |
| 94924_NCI-H157_Squa- | 0.0 | 126768_293 cells | 12.6 |
| mous cell lung cancer | | | |
| (metastasis) | | | |
| 94925_NCI-H1155_Large | 100.0 | 94987_Hs766T_Pan- | 0.0 |
| cell lung cancer/ | | creatic carcinoma | |
| neuroendocrin | | (LN metastasis) | |
| 94926_NCI-H1299_Large | 0.2 | 94988_CAPAN-1_Pan- | 0.0 |
| cell lung cancer/ | | creatic adenocarcinoma | |
| neuroendocrine | | (liver metastasis) | |
| 94927_NCI-H727_Lung | 0.0 | 94989_SU86.86_Pan- | 0.0 |
| carcinoid | | creatic carcinoma (liver | |
| | | metastasis) | |
| 94928_NCI-UMC-11_Lung | 0.9 | 94990_BxPC-3_Pan- | 0.0 |
| carcinoid | | creatic adenocarcinoma | |
| 94929_LX-1_Small cell | 0.0 | 94991_HPAC_Pancreatic | 0.0 |
| lung cancer | | adenocarcinoma | |
| 94930_Colo-205_Colon | 2.5 | 94992_MIA PaCa-2_Pan- | 0.0 |
| cancer | | creatic carcinoma | |
| 94931_KM12_Colon | 0.0 | 94993_CFPAC-1_Pan- | 0.0 |
| cancer | | creatic ductal | |
| | | adenocarcinoma | |
| 94932_KM20L2_Colon | 0.0 | 94994_PANC-1_Pan- | 0.1 |
| cancer | | creatic epitheliold | |
| | | ductal carcinoma | |
| 94933_NCI-H716_Colon | 0.6 | 94996_T24_Bladder | 0.0 |
| cancer | | carcinma (transitional cell) | |
| 94935_SW-48_Colon | 0.4 | 94997_5637_Bladder | 0.0 |
| adenocarcinoma | | carcinoma | |
| 94936_SW1116_Colon | 0.0 | 94998_HT-1197_Bladder | 0.0 |
| adenocarcinoma | | carcinoma | |
| 94937_LS 174T_Colon | 0.0 | 94999_UM-UC-3_Bladder | 0.0 |
| adenocarcinoma | | carcinma (transitional cell) | |
| 94938_SW-948_Colon | 0.3 | 95000_A204_Rhab- | 0.5 |
| adenocarcinoma | | domyosarcoma | |
| 94939_SW-480_Colon | 0.0 | 95001_HT-1080_Fibro- | 0.0 |
| adenocarcinoma | | sarcoma | |
| 94940_NCI-SNU-5_Gastric | 0.4 | 95002_MG-63_Osteo- | 0.0 |
| carcinoma | | sarcoma (bone) | |
| 112197_KATO III_Stomach | 0.0 | 95003_SK-LMS-1_Leio- | 0.0 |
| | | myosarcoma (vulva) | |
| 94943_NCI-SNU-16_Gastric | 0.0 | 95004_SJRH30_Rhab- | |
| carcinoma | | domyosarcoma (met to | |
| | | bone marrow) | |
| 94944_NCI-SNU-1_Gastric | 0.0 | 95005_A431_Epidermoid | 0.0 |
| carcinoma | | carcinoma | |
| 94946_RF-1_Gastric | 0.2 | 95007_WM266-4 | 0.0 |
| adenocarcinoma | | Melanoma | |
| 94947_RF-48_Gastric | 0.0 | 112195_DU 145_Prostate | 0.4 |
| adenocarcinoma | | | |
| 96778_MKN-45_Gastric | 1.2 | 95012_MDA- | 0.0 |
| carcinoma | | MB-468_Breast | |
| | | adenocarcinoma | |
| 94949_NCI-N87_Gastric | 0.0 | 112196_SSC-4_Tongue | 0.0 |
| carcinoma | | | |
| 94951_OVCAR-5_Ovarian | 0.0 | 112194_SSC-9_Tongue | 0.0 |
| carcinoma | | | |
| 94952_RL95-2_Uterine | 0.0 | 112191_SSC-15_Tongue | 0.0 |
| carcinoma | | | |
| 94953_HelaS3_Cervical | 0.2 | 95017_CAL 27_Squa- | 0.0 |
| adenocarcinoma | | mous cell carcinoma of | |
| | | tongue | |

Ardais Breast 1.0 Summary: Ag7884 Expression of the CG54020 gene was highest in a breast cancer sample (CT=28.4). In general, expression of this gene was higher in breast tumors than in normal breast tissue. The breast cancer samples (N=64) had an average CT of 35.3 (±3.2), whereas the normal breast samples (N=7) had an average CT of 37.6 (±3.4). The number of cancer samples with CT values less than the average normal CT value minus 2 standard deviations was 8/64; in contrast, 0/7 normal samples had CT values less than the average minus 2 standard deviations. Therefore, expression of this gene or its protein product is useful as a marker to detect breast cancer. Furthermore, gene, protein, antibody or small molecule therapeutics targeting this gene or its protein product could be useful in the treatment of breast cancer.

Ardais Panel 1.1 Summary: Ag7884 Expression of the CG54020 gene was highest in a lung cancer sample (CT=26.3). In general, expression of this gene was higher in lung tumors than in normal lung tissue. The lung cancer samples (N=13) had an average CT of 31.8 (±3.1), whereas the normal lung samples (N=8) had an average CT of 35.1 (±1.2). The number of cancer samples with CT values less than the average normal CT value minus 2 standard deviations was 7/13; in contrast, 1/8 normal samples had CT values less than the average minus 2 standard deviations. Therefore, expression of this gene or its protein product is useful as a marker to detect lung cancer. Furthermore, gene, protein, antibody or small molecule therapeutics targeting this gene or its protein product could be useful in the treatment of lung cancer.

General_screening_panel_v1.7Summary: Ag7884 Expression of the CG54020 gene was highest in lung cancer cell line SHP-77 (CT=25.4). This gene was expressed at moderate levels in 5 out of 7 of the additional lung cancer cell lines tested whereas no significant expression was seen in normal adult or fetal lung. These results are consistent with what was observed in Ardais Panel v1.1.

The CG54020 gene was also overexpressed in 2 out of 5 breast cancer cell lines when compared to normal breast, consistent with what was observed in the Ardais Breast Panel v1.0. Furthermore, this gene was overexpressed in 2/6 ovarian cancer cell lines as well as 2/9 colon cancer cell lines when compared to the appropriate normal controls.

Therefore, expression of this gene or its protein product is useful as a marker to detect lung, breast, ovarian and colon cancer. Furthermore, gene, protein, antibody or small molecule therapeutics targeting this gene or its protein product could be useful in the treatment of lung, breast, ovarian and colon cancer.

Among normal tissues, expression of this gene was highest in the brain (CTs=28.6 to 32.4). Significant expression was also detected in adrenal gland, pituitary gland, and spleen.

Oncology_cell_line_screening_panel_v3.2 Summary: Ag7884 Expression of the CG54020-01 gene was highest in large cell lung cancer cell line NCI-H1155 (CT=28.6). Significant expression of this gene was also seen in 6/7 small cell lung cancer cell lines, consistent with was observed in Panel 1.7 and Ardais v1.1. This gene was also expressed at moderate levels in a medulloblastoma cell line.

Conclusions

The RTQ-PCR results from primary lung tumors (Ardais Panel v1.1) indicate that CG54020 overexpression was clustered toward the adenocarcinomas (stage I and II). NSCLCs are approximately equally divided between the two major histological subtypes, adenocarcinoma and squamous cell carcinoma (˜70% of total cases). Adenocarcinoma is prevalent in women smokers, occurs in peripheral lung tissue and has a predilection to disseminate. In contrast, squamous cell carcinoma (SCC) affects primarily men and typically arises in the deep periphery of the lung with a tendency to encapsulate and involute.

Samples from large cell NSCLC (˜10% of total cases), the most aggressive and drug resistant NSCLC subtype, were not represented on the Ardais Panel v1.1. Small cell carcinomas (˜20% of total cases) were also not represented on this panel; these tumors are typically more chemotherapy sensitive, tend to be large central masses with almost guaranteed extensive mediatinal node involvement, and approximately two-thirds show visceral metastasis at the time of diagnosis. However, the CG54020 gene tends to be highly expressed in cell lines derived from small cell carcinoma and large cell NSCLC (Panels 1.7 and 3.2). Therefore, CG54020 appears to be overexpressed in lung cancers independent of the type. Thus, expression of this gene or its protein product is an attractive marker to detect lung cancer. Furthermore, gene, protein, antibody or small molecule therapeutics targeting this gene or its protein product could be useful in the treatment of lung cancer.

CG54020 was also overexpressed in breast tumors and breast cancer cell lines. We have identified a number of proteins that interact with CG54020 whose expression is also upregulated in breast cancer and that are known to play a role in the disease (Example 7). Thus, expression of CG54020 or its protein product is an attractive marker to detect breast cancer. Furthermore, gene, protein, antibody or small molecule therapeutics targeting this gene or its protein product could be useful in the treatment of breast cancer.

### EXAMPLE 7

Pathways Relevant to the Etiology and Pathogenesis of Lung and/or Breast Cancer

Materials and Methods

PathCalling™ Technology: The sequence of Acc. No CG54020-01 was derived by laboratory screening of cDNA library by the two-hybrid approach. cDNA fragments covering either the full length of the DNA sequence, or part of the sequence, or both, were sequenced. In silico prediction was based on sequences available in CuraGen Corporation's proprietary sequence databases or in the public human sequence databases, and provided either the full-length DNA sequence, or some portion thereof.

The laboratory screening was performed using the methods that follow. cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Interacting protein pairs are added to CuraGen's PathCalling™ Protein Interaction Database. This database allows for the discovery of novel pharmaceutical drug targets by virtue of their interactions and/or presence in pathologically related signaling pathways. Protein interactions are subsequently analyzed using bioinformatic tools within GeneScape™, which provides a means of visualization of binary protein interactions, protein complex formation, as well as complete cellular signaling pathways.

Physical clone: The cDNA fragment derived by the screening procedure, covering the entire open reading frame was cloned into pACT2 plasmid (Clontech) used to make the cDNA library. The recombinant plasmid was inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

Results and Discussion:

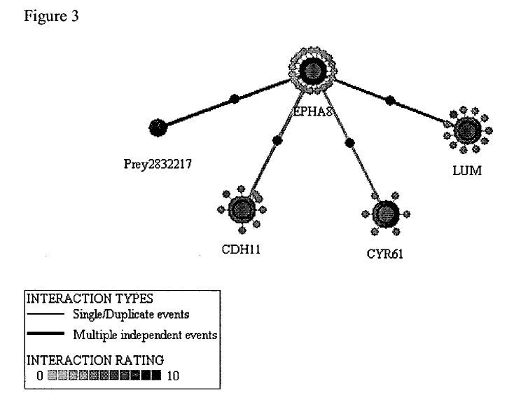

CG54020 (EPHA8) had a number of high confidence and significant interactors. Specifically, as shown in FIG. 3, the sequences that encode proteins CG54020 (EPHA8), LUM, CDH11, CYR61 and Prey2832217 proteins were found to interact and can result in the formation of a protein complex, or may constitute a series of complexes, which form in order to propagate a cellular signal, which is physiologically relevant to a disease pathology. The specific interactions, which constitute the specific complexes, may also be useful for therapeutic intervention through the use of recombinant protein or antibody therapies, small molecule drugs, or gene therapy approaches. Three of these interactors, namely LUM (lumican), CDH11 (cadherin 11) and CYR61, have elevated expression in breast cancer cells, like CG54020, and are additionally implicated in breast tumor cell invasion or progression.

Lumican (338 aa; NP_{—}002336) is an extracellular matrix protein from the small leucine-rich proteoglycan family that functions in cell migration, proliferation, and extracellularmatrix modeling. Lumican is highly expressed in breast tumors relative to normal breast tissue and is implicated in breast tumor progression (Leygue et al. Cancer Res 1998 58:1348-52; Leygue et al. J Pathol 2000 192:313-20). By PathCalling, lumican interacted with Ephrin receptor A8 and IGF-binding protein FKSG28. The interaction between CG54020 and lumican had an extremely high interaction rating score (9.99), making it a high confidence interaction. Table 4 summarizes the amino acid sequences of the bait and prey used in forty-eight independent experiments to detect this novel interaction. EPHA8, like lumican, is overexpressed in breast cancers and signaling through the receptor is known to induce cell migration (Gu and Park, FEBS Lett 2003 540:65-70).

It is hypothesized that since lumican is a proteoglycan it may bind growth factors, bringing them to the tumor resulting in tumor cell proliferation. The interaction with IGF-binding protein supports the hypothesis. IGF-binding proteins are highly overexpressed in many different types of cancers and enhance IGF-receptor signaling by increasing the local concentration of IGF. Similarly, lumican may also bind growth factors such as ephrins, resulting in the activation of Ephrin receptors.

TABLE 4
Eph A8 Receptor Yeast Two-hybrid Interaction Information

| | Eph A8 | | |
|---|---|---|---|
| Interaction | Interaction | Prey Interaction | Number of Yeast |
| Frame | Domain (aa) | Domain (aa) | Colonies Observed |
| 1 (+) | 1-180, | LUM: 217-338 to | 145 |
| | 1-231 | 307-338 | |
| 1 (+) | 1-180 | CDH11: 154-796 | 3 |
| 1 (+) | 1-515 | CYR61: 79-381 | 1 |
| 1 (+) | 1-180 | Prey 2832217: −35-167 | 31 |

The ephrin A8 receptor was found to interact with CDH11, with an interaction rating score of 2.7 (Table 4). Cadherin 11 (796 aa; NP_{—}001788) is overexpressed in invasive breast cancer cell lines (Pishvaian et al., Cancer Res 1999 59:947-52). CuraChip data also indicated relative high expression of CDH11 in breast tumor samples. In addition, a splice variant of cadherin-11 has been shown to promote invasion of cadherin-11 positive breast cancer cells (Feltes et al., Cancer Res 2002 62:6688-97).

Furthermore, CDH11-mediated adhesion has been shown to induce expression of the angiogenic factor VEGFD (Orlandini and Oliviero, J Biol Chem 2001 276:6576-81). Thus, the interaction between CG54020 and cadherin 11 may play a role in cell migration and angiogenesis.

CG54020 was also found to interact with the CYR61 protein (Table 4). CYR61 (381 aa; NP_{—}001545), an angiogenic regulator, is overexpressed in invasive and metastatic human breast cancer cells and tumor biopsies (Tsai et al., Oncogene 2002 21:964-73).

Finally, CG54020 was also found to interact with the Prey 2832217 protein (Table 4). Prey 2832217 encodes the C11orf15 protein (198 aa; Q9NQ34), a protein of unknown function that has one CXCXC motif, a motif also found in VEGFC. The hydropathy plot of Prey 2832217 suggests that it encodes a single-pass transmembrane protein. The interaction between CG54020 and Prey 2832217 had an extremely high interaction rating score (9.6), making it a high confidence interaction.

In conclusion, the pathway interaction results together with the overexpression of several of these genes in breast cancer support a role for CG54020 in breast tumor vascularization and invasion.

### EXAMPLE 8

Generation and Characterization of Monoclonal Antibodies that Bind CG54020

Techniques for producing the antibodies are known in the art and are described, for example, in “Antibodies, a Laboratory Manual” Eds Harlow and Lane, Cold Spring Harbor publisher. Both rabbits and mice are suitable for the production of polyclonal antibodies, while mice are also suitable for the production of monoclonal antibodies. Mice in which the human immunoglobulin genes replace mouse immunoglobulin genes can be used to produce fully human monoclonal antibodies. These antibodies have better pharmaceutical characteristics, have little or no antibody-directed immune reactions that result in loss of therapeutic efficacy, and have been shown to eradicate tumors in animal model (Yang et al., Cancer Res 1999 59:1236-43).

Materials and Methods

Expression and Purification of CG54020-02 Antigen: Multiple batches of CG54020-02 were purified from HEK293 cells using metal affinity chromatography (Pharmacia). The CG54020-02 antigen protein was eluted with a linear gradient 50-500 mM imidazole. The fractions containing the CG54020-02 protein were concentrated 2000-fold by dialysis against 20 mM Tris-HCl, 50 mM NaCl pH 7.4 using a 3500 MW cutoff dialysis membrane (taken from DD CoA batch 2).

The CG54020-02 protein was electrophoretically transferred to a polyvinylidenefluoride membrane and the stained 66 kilodalton band was excised from the membrane and analyzed by an automated Edman sequencer (Procise, Applied Biosystems, Foster City, Calif.). The N-terminal amino acid sequence of the first 8 amino acids was confirmed as identical to the predicted protein sequence.

Generation of Human Monoclonal Antibodies: Fully human IgG1 monoclonal antibodies (mAb), directed against CG54020-02 are generated using standard hybridoma technology or from human antibody-producing XenoMouse strains engineered to be deficient in mouse antibody production and to contain the majority of the human antibody gene repertoire on megabase-sized fragments from the human heavy and kappa light chain loci as previously described in Yang et al., Cancer Res 1999 59:1236-43.

ELISA: The monoclonal antibodies generated by using the above technique are titrated against G54020-02 by using standard ELISA assay known in the art.

Epitope Binding: To determine if the epitope binds to the antibodies generated, following protocol is used. MxhIgG-conjugated beads are coupled to primary unknown antibody. A 96-well microtiter filter plate (Millipore, Billerica, Mass.) is pre-wet by adding 200 μl wash buffer (PBS, Tween 20 {0.05%}) per well and aspirating. A 50 μl aliquot of each bead sample is added to the filter plate wells and washed once with wash buffer. 50 μl antigen and controls are added to each well and incubated for 1 h at room temperature. After washing 3 times with wash buffer, secondary unknown antibody is added at 50 μl/well using the same dilution (or concentration if known) as used for the primary antibody. The plate is incubated for 2 h at room temperature with shaking. After washing 3 times with wash buffer, 50 μl biotinylated mxhIgG diluted 1:500 is added to each well and incubated for 1 h at room temperature with shaking. After washing 3 times with wash buffer, 50 μl/well Streptavidin-PE diluted 1:1000 is added to each well incubated for 15 min at room temperature with shaking. After washing 3 times with wash buffer, 80 μl blocking buffer is used to resuspend the bead samples. Samples are analyzed on a Luminex 100 (Luminex, Austin, Tex.).

Neutralization Assay: In order to test whether a given monoclonal antibody had neutralizing activity and thus could block the function of the Ephrin A8 receptor, neutralization experiments are performed using Ephrin A4 ligand. The ability of each monoclonal antibody to block CG54020-02 protein interaction with ephrin A4 ligand is be measured.

Internalization Assay: Antibodies that are internalized in cells are potential candidates for toxin-killing approaches. Therefore, the CG54020 monoclonal antibodies are tested for ability to internalize using a fluorescent detection kit. For adherent cell lines, cells are removed from the plate using Cell Dissociation Buffer (Sigma, St. Louis, Mo.). Cells are harvested and washed with 10 mL of ice-cold FACs buffer (PBS+10% FBS); 2×10⁵ cells per well are transferred to a V-bottom 96-well plate. Next, 100 μL of primary monoclonal antibody is added to each well at 1 μg/mL and incubated for 20 minutes at 4° C. Cells are pelleted and washed once with 200 μL of ice-cold FACs buffer. 100 μL of Reagent A is added to each well and incubated for 7 minutes at 4° C. Cells are then pelleted and washed once with 200 μL of ice-cold FACs buffer. Cells are incubated at 4° C. or 37° C. for 30 minutes. Cells are pelleted and internalization is stopped by the addition of 200 μL of ice-cold FACs buffer or 200 μL of ice-cold freshly made Reagent B. Cells are incubated at 37° C. for 30 minutes. Finally, cells are pelleted, washed once with 200 μL of ice-cold FACs buffer and analyzed by flow cytometry.

The percent internalization is determined using the following formula: Percent internalization=(a−b)/(c−b)×100, where a=mean fluorescence at 37° C. with Reagent B, b=mean fluorescence at 4° C. with Reagent B, and c=mean fluorescence at 37° C. without Reagent B.

BIAcore Affinity Determination: BIAcore (KD) determinations are done using methods known in the art, for example, Wong et al., Journal of Immunological Methods 1997209: 1-15.

Conclusion

Initial ELISAs are performed on all mAbs using purified CG54020-02 protein (the antigen) or V5-His peptide. Those antibodies that exhibit a positive reaction with the CG54020-02 protein and a negative reaction with V5-His peptide are selected for subsequent use in FACs analysis. Epitope binning is performed on a subset of CG54020 monoclonal antibodies that is positive with ELISA.

The antibodies will be further tested for internalization by cancer cell lines as CG54020 is highly expressed in cancer cells and tissues as shown by RTQ-PCR results (Example 6). Internalizing monoclonal antibodies will further be used as candidates for treatment of cancer using a toxin-conjugate approach.

### EXAMPLE 9

Quantification of Membrane Bound CG54020 Protein by Flow Cytometry

Flow cytometry analysis is performed to demonstrate the specificity of the anti-CG54020 antibodies for cell membrane-bound CG54020 and to identify preferred antibodies for use as a therapeutic or diagnostic agent.

Materials and Methods

Flow Cytometry: FACs analysis is performed on lung, breast and brain cancer cell lines based on the results showing increased expression of CG54020 antigen by RTQ-PCR (Patent Example 6). Negative controls are NCI-H292 and HOP-62 that were lacking expression of CG54020 by RTQ-PCR (CT value=40). Cells are washed with Ca and Mg-free 1×PBS (Media Tech, MT 21-040-CV). Versene (Invitrogen 15040-066) is added and the cells incubated at 37° C. until they detached. Cells are counted and 500,000 to 1,000,000 cells/tube are used for FACS analysis. Cells are washed twice with ice-cold FACS buffer (1×PBS, 4% FBS) and resuspended in 100 μL monoclonal antibody at 1 μg/mL. Cells are mixed and incubated at 4° C. or on ice for 30 min. Cells are washed twice with 1 mL ice-cold FACs buffer and secondary conjugated antibody is added. Cells are incubated at 4° C. or on ice for 30 min. Cells are washed twice with 1 mL ice-cold FACS buffer and fixed with 400-500 mL 1% formaldehyde in PBS (Sigma F 1635).

### EXAMPLE 10

Indirect Toxin-Killing of CG54020 Positive Cancer Cell Lines

CG54020 was overexpressed in a number lung and breast cancer cell lines and tumors as shown by RTQ-PCR results (patent example 6). Therefore, it will be tested whether the CG54020 mAbs can induce cancer cell death when used in combination with a toxin-conjugated secondary antibody reagent. The secondary (indirect) reagent employed utilizes the toxin saporin. At the concentration used, saporin must be internalized to induce cell death. Following internalization, saporin dissociates from its carrier antibody and translocates to the cytoplasm where it inhibits protein synthesis, an outcome ultimately leading to cell death (Kohls and Lappi, Biotechniques 2000 28:162-5).

Materials and Methods

Cell Titer Blue and Clonogenic Assays: Cells are plated in 100 μl/well growth media (RPMI or DMEM+10% FBS) in 96-well flat bottom tissue culture plates at a concentration that will give rise to 25% confluency on Day 2; 3 wells with no cells are included as blank control for the Cell Titer Blue assay. Cells are plated in duplicate; one plate is used for the Cell Titer Blue assay and the other for the clonogenic assay on day 5. Cells are incubated at 37° C. overnight.

On Day 2, the indirect toxin reagent (Advanced Targeting Systems, San Diego, Calif.) is diluted in growth media to a concentration of 4.0 μg/mL and 25 μl is added to each well such that the final amount of reagent utilized is 100 ng/well. Primary antibody (6× stock) is diluted in growth media to desired concentrations and 25 μL/well is added to the cells. Controls include: primary antibody without indirect toxin reagent (added 25 μL/well growth media instead of 25 μL/well of diluted indirect toxin reagent); indirect toxin reagent without primary antibody (added 25 μL/well growth media instead of 25 μL/well diluted primary antibody); no primary antibody and no indirect toxin reagent (added 50 μL/well growth media); and control primary antibody pK16.3. Cells are then incubated at 37° C. for 3 days.

On Day 5, cells are visually inspected for evidence of cell killing. Using one of the duplicate cell plates, a Cell Titer Blue assay is done according to the manufacturer's instructions (Promega, Madison, Wis.; Cat #G8081) using fluorescence readout. Using the other duplicate plate, a clonogenic assay is performed using techniques known in the art. For example, Kohls and Lappi (Biotechniques, 2000 28:162-5) have described a clonogenic assay to determine if a primary antibody can induce cancer cell death when used in combination with a saporin toxin conjugated secondary antibody reagent. Briefly, medium is removed from the 96-well dish and the cells from each well washed, trypsinized, and transferred to a single well of a 6-well dish or to a 100 mm dish containing growth media. Cells are incubated until colonies are sufficient size for counting, feeding every 3-4 days with fresh growth media.

Conclusion

The results obtained using the Cell Titer Blue and clonogenic assays should be comparable and will indicate the ability of CG54020 monoclonal antibodies to induce cancer cell death. Of particular interest is the activity of the CG54020 antibodies in inducing lung and breast cancer cell death.

### EXAMPLE 11

Preparation and Testing of Chemotherapy and Radio-Immunoconjugated Antibodies

Cytotoxic chemotherapy or radiotherapy of cancer is limited by serious, sometimes life-threatening side effects that arise from toxicities to sensitive normal cells because the therapies are not selective for malignant cells. Therefore, there is a need to improve the selectivity. One strategy is to couple therapeutics to antibodies that recognize tumor-associated antigens. This increases the exposure of the malignant cells to the ligand-targeted therapeutics but reduces the exposure of normal cells to the same agent. (reviewed in Allen, Nat Rev Cancer, 2002 2:750-63).

CG54020 is one of these tumor-associated antigens, as shown by its specific expression on cellular membranes of tumor cells by FACS. Therefore one embodiment of the invention uses monoclonal antibodies directed against CG54020 coupled to cytotoxic chemotherapic agents or radiotherapic agents as anti-tumor therapeutics.

Depending on the intended use of the antibody, i.e., as a diagnostic or therapeutic reagent, radiolabels are known in the art and have been used for similar purposes. For instance, radionuclides that have been used in clinical diagnosis include I¹³¹, I¹²⁵, I¹²³, Tc⁹⁹, Ga⁶⁷, as well as In¹¹¹. Antibodies have also been labeled with a variety of radionuclides for potential use in targeted immunotherapy (Peitersz et al. Immunol. Cell Bio, 1987 165: 111-125). These radionuclides include Re¹⁸⁸ and Re¹⁸⁶ as well as Y⁹⁰, and to a lesser extent Au¹⁹⁹ and Cu⁶⁷. I¹³¹ has also been used for therapeutic purposes. U.S. Pat. No. 5,460,785 provides a listing of such radioisotopes.

Radiotherapeutic chelators and chelator conjugates are known in the art. For instance, U.S. Pat. No. 4,831,175 is directed to polysubstituted diethylenetriaminepentaacetic acid chelates and protein conjugates containing the same, and methods for their preparation. U.S. Pat. Nos. 5,099,069; 5,246,692; 5,286,850; and 5,124,471 also relate to polysubstituted DTPA chelates.

Cytotoxic chemotherapies are known in the art and have been used for similar purposes. For instance, U.S. Pat. No 6,441,163 describes processes for the production of cytotoxic conjugates of maytansinoids and antibodies. The anti-tumor activity of a new tubulin polymerization inhibitor, auristatin PE, is also know in the art (Mohammad et al., Int J Oncol, 1999 15:367-72).

Once these chemotherapy or radiolabel and antibody conjugates are made, they can be tested for their cytotoxic activity on CG54020 expressing cells. Such experiments use methods known in the art, such as MTS, cell counting and clonogenic assays.

Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. The claims presented are representative of the inventions disclosed herein. Other, unclaimed inventions are also contemplated. Applicants reserve the right to pursue such inventions in later claims.

What is claimed is:

1. A method of treating, preventing, or delaying a cell proliferation-associated disorder comprising administering to a subject a therapeutically effective amount of an antibody that binds immunospecifically to a polypeptide selected from the group consisting of:
a) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
b) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
c) the polypeptide of (a) and (b), wherein one or more amino acid substitutions are made to the polypeptide to produce a variant, provided that the variant is no more than 15% divergent in sequence from the polypeptide, and provided that said variant retains cellular proliferation modulatory activity;
d) a fragment of the polypeptide of (a), (b), or (c), which fragment retains cellular proliferation modulatory activity; and
e) a fragment of the polypeptide of (a), (b), or (c), which fragment retains kinase activity.

2. The method of claim 1, wherein the subject is a mammal.

3. The method of claim 2, wherein the mammal is a human.

4. The method of claim 1, wherein the cell proliferation-associated disorder is lung cancer, breast cancer, or a cancer of the nervous system.

5. The method of claim 1, wherein the cell proliferation-associated disorder is selected from the group consisting of lung cancer, metastatic lung cancer, lung adenocarcinoma, small cell lung cancer, squamous cell lung carcinoma, large cell carcinoma, adenosquamous carcinoma, undifferentiated lung carcinoma, breast cancer, infiltrating ductal carcinoma, metastatic breast cancer, and brain cancer.

6. The method of claim 1, wherein said antibody is a polyclonal antibody, a monoclonal antibody, or a humanized monoclonal antibody.

7. The method of claim 1, wherein administering comprises providing said antibody to the subject intravenously.

8. The method of claim 1, wherein administering comprises providing said antibody to the subject parenterally.

9. A purified antibody that binds immunospecifically to a polypeptide selected from the group consisting of:
a) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
b) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
c) the polypeptide of (a) and (b), wherein one or more amino acid substitutions are made to the polypeptide to produce a variant, provided that the variant is no more than 15% divergent in sequence from the polypeptide, and provided that said variant retains cellular proliferation modulatory activity;
d) a fragment of the polypeptide of (a), (b), or (c), which fragment retains cellular proliferation modulatory activity; and
e) a fragment of the polypeptide of (a), (b), or (c), which fragment retains kinase activity.

10. The antibody of claim 9, wherein said antibody is a human monoclonal antibody.

11. The antibody of claim 9, wherein said antibody is conjugated to a conjugation agent.

12. The antibody of claim 11, wherein said conjugation agent is a chemotherapic agent or a radiotherapic agent.

13. The antibody of claim 12, wherein said chemotherapic agent is selected from the group consisting of diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins, momordica charantia inhibitor, curcin, crotin, Sapaonaria officinalis inhibitor, gelonin, mitogellin, restrictocin, phenomycin, enomycin, and a tricothecene.

14. The antibody of claim 11, wherein said conjugation agent is an antibody conjugated to a toxin.

15. The antibody of claim 11, wherein said conjugation agent is a detectable entity.

16. An isolated polypeptide comprising an amino acid sequence selected from the group consisting of:
a) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
b) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
c) the polypeptide of (a) and (b), wherein one or more amino acid substitutions are made to the polypeptide to produce a variant, provided that the variant is no more than 15% divergent in sequence from the polypeptide, and provided that said variant retains cellular proliferation modulatory activity;
d) a fragment of the polypeptide of (a), (b), or (c), which fragment retains cellular proliferation modulatory activity; and
e) a fragment of the polypeptide of (a), (b), or (c), which fragment retains kinase activity.

17. The polypeptide of claim 16, wherein said polypeptide comprises the amino acid sequence of a naturally-occurring allelic variant of an amino acid sequence selected from the group consisting SEQ ID NOS: 2, 4, 6, 8, 10, 12, 14, 16, 18, 20, 22, 24, 26, 28, 30, 32, 34, and 36.

18. The polypeptide of claim 16, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, 31, 33 and 35.

19. The polypeptide of claim 16, wherein the amino acid sequence of said variant comprises a conservative amino acid substitution.

20. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, and 36;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, and 36, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, and 36;
(d) a variant of an amino acid sequence selected from the group consisting SEQ ID NOS: 2, 4, 6, 8, 10, 12, 14, 16, 18, 20, 22, 24, 26, 28, 30, 32, 34, and 36, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28, 30, 32, 34, and 36, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

21. The nucleic acid molecule of claim 20, wherein the nucleic acid molecule comprises the nucleotide sequence of a naturally-occurring allelic nucleic acid variant.

22. The nucleic acid molecule of claim 20, wherein the nucleic acid molecule encodes a polypeptide comprising the amino acid sequence of a naturally-occurring polypeptide variant.

23. The nucleic acid molecule of claim 20, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, 25, 27, 29, 31, 33 and 35.

24. The nucleic acid molecule of claim 20, wherein said nucleic acid molecule hybridizes under stringent conditions to a nucleotide sequence chosen from the group consisting of 1, 3, 5, 7, 9, 11, 13, 15, 17, 19, 21, 23, 25, 27, 29, 31, 33 and 35, or a complement of said nucleotide sequence.

25. The nucleic acid molecule of claim 20, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

26. A vector comprising the nucleic acid molecule of claim 20.

27. The vector of claim 26, further comprising a promoter operably-linked to said nucleic acid molecule.

28. A cell comprising the vector of claim 27.

29. A method of preparing a pharmaceutical composition comprising combining at least one antibody effective in treating, preventing, or delaying a cell proliferation-associated disorder with a pharmaceutically acceptable carrier, wherein said antibody binds immunospecifically to a polypeptide selected from the group consisting of:
a) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
b) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
c) the polypeptide of (a) and (b), wherein one or more amino acid substitutions are made to the polypeptide to produce a variant, provided that the variant is no more than 15% divergent in sequence from the polypeptide, and provided that said variant retains cellular proliferation modulatory activity;
d) a fragment of the polypeptide of (a), (b), or (c), which fragment retains cellular proliferation modulatory activity; and
e) a fragment of the polypeptide of (a), (b), or (c), which fragment retains kinase activity.

30. The method of claim 29, wherein the cell proliferation-associated disorder is breast cancer, lung cancer, or a cancer of the nervous system.

31. The method of claim 29, wherein the pharmaceutical composition is suitable for intravenous, subcutaneous, or parenteral administration to a subject.

32. The method of claim 29, wherein the subject is a mammal.

33. The method of claim 32, wherein the mammal is a human.

34. A method for determining the presence of or predisposition to a cell proliferation-associated disorder associated with altered levels of a polypeptide selected from the group consisting of:
a) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
b) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
c) the polypeptide of (a) and (b), wherein one or more amino acid substitutions are made to the polypeptide to produce a variant, provided that the variant is no more than 15% divergent in sequence from the polypeptide, and provided that said variant retains cellular proliferation modulatory activity;
d) a fragment of the polypeptide of (a), (b), or (c), which fragment retains cellular proliferation modulatory activity; and
e) a fragment of the polypeptide of (a), (b), or (c), which fragment retains kinase activity
in a first mammalian subject, the method comprising:
i) measuring the amount of the polypeptide in a sample from the first mammalian subject using an antibody that immunospecifically binds to the polypeptide; and
ii) comparing the amount of the polypeptide in the sample of step (i) to the amount of the polypeptide present in a control sample from a second mammalian subject known not to have, or not to be predisposed to, the disorder;
wherein an alteration in the level of the polypeptide in the first subject as compared to the control sample indicates the presence of or predisposition to the disorder.

35. A drug formulation for treating, preventing, or delaying a cell proliferation-associated disorder in a subject comprising:
a) a therapeutically effective amount of an antibody that immunospecifically binds a polypeptide selected from the group consisting of:
i) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
ii) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
iii) the polypeptide of (i) and (ii), wherein one or more amino acid substitutions are made to the polypeptide to produce a variant, provided that the variant is no more than 15% divergent in sequence from the polypeptide, and provided that said variant retains cellular proliferation activity;
iv) a fragment of the polypeptide of (i), (ii), or (iii), which fragment retains cellular proliferation activity; and
v) a fragment of the polypeptide of (a), (b), or (c), which fragment retains kinase activity, and
b) a formulation buffer.

36. A method of modulating the proliferation of a mammalian cell comprising contacting the cell with an antibody that immunospecifically binds to a polypeptide selected from the group consisting of:
i) a polypeptide comprising the amino acid sequence selected from the group consisting of SEQ ID NOS: 2, 4, 6, 8, 10, 12, 14, 16, 18, 20, 22, 24, 26, 28,30, 32, 34, and 36;
ii) a mature form of a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
iii) the polypeptide of (i) and (ii), wherein one or more amino acid substitutions are made to the polypeptide to produce a variant, provided that the variant is no more than 15% divergent in sequence from the polypeptide, and provided that said variant retains cellular proliferation activity;
iv) a fragment of the polypeptide of (i), (ii), or (iii), which fragment retains cellular proliferation modulatory activity.

37. The method of claim 36, wherein the polypeptide or fragment has at least one property selected from the group consisting of:
a) increasing the proliferation of a mammmalian cell; and
b) increasing the growth of mammalian cell.

38. The method of claim 36, wherein the polypeptide or fragment has at least one property selected from the group consisting of:
a) decreasing the proliferation of a mammmalian cell; and
b) decreasing the growth of mammalian cell.

39. The method of claim 36, wherein the mammalian cell is of mesenchymal, epithelial, or endothelial origin.

40. A method of modulating blood vessel formation in a mammal, comprising contacting the mammal with an antibody that immunospecifically binds to a polypeptide selected from the group consisting of:
i) a polypeptide comprising the amino acid sequence selected from the group consisting of SEQ ID NOS: 2, 4, 6, 8, 10, 12, 14, 16, 18, 20, 22, 24, 26, 28, 30, 32, 34, and 36;
ii) a mature form of a polypeptide comprising the amino acid sequence selected from the group consisting of SEQ ID NOS: 2, 4, 6, 8, 10, 12, 14, 16, 18, 20, 22, 24, 26, 28, 30, 32,34, and 36;
iii) the polypeptide of (i) and (ii), wherein one or more amino acid substitutions are made to the polypeptide to produce a variant, provided that the variant is no more than 15% divergent in sequence from the polypeptide, and provided that said variant retains cellular proliferation activity;
iv) a fragment of the polypeptide of (i), (ii), or (iii), which fragment retains cellular proliferation modulatory activity;
v) a fragment of the polypeptide of (i), (ii), or (iii), which fragment retains kinase activity.

41. The method of claim 40, wherein said blood vessel formation is selected from the group consisting of angiogenesis and vasculogenesis.

42. The method of claim 40, wherein said blood vessel formation is induced by a tumor cell.

43. The method of claim 16, wherein the polypeptide further comprises a post-translational modification.

44. The method of claim 43, wherein the post-translational modification is at least one modification chosen from the group consisting of phosphorylation, glycosolation, and N-myristoylation.


SEQUENCE LISTING (41 sequences)

SEQ ID NO: 1 - DNA, 3018 bp, Homo sapiens
  CDS (1)..(3015)
atggcccccgcccggggccgcctgccccctgcgctctgggtcgtcacggccgcggcggcggcggccacctgcgtgtccgcggcgcgcggcgaagtgaatttgctggacacgtcgaccatccacggggactggggctggctcacgtatccggctcatgggtgggactccatcaacgaggtggacgagtccttccagcccatccacacgtaccaggtttgcaacgtcatgagccccaaccagaacaactggctgcgcacgagctgggtcccccgagacggcgcccggcgcgtctatgctgagatcaagtttaccctgcgcgactgcaacagcatgcctggtgtgctgggcacctgcaaggagaccttcaacctctactacctggagtcggaccgcgacctgggggccagcacacaagaaagccagttcctcaaaatcgacaccattgcggccgacgagagcttcacaggtgccgaccttggtgtgcggcgtctcaagctcaacacggaggtgcgcagtgtgggtcccctcagcaagcgcggcttctacctggccttccaggacataggtgcctgcctggccatcctctctctccgcatctactataagaagtgccctgccatggtgcgcaatctggctgccttctcggaggcagtgacgggggccgactcgtcctcactggtggaggtgaggggccagtgcgtgcggcactcagaggagcgggacacacccaagatgtactgcagcgcggagggcgagtggctcgtgcccatcggcaaatgcgtgtgcagtgccggctacgaggagcggcgggatgcctgtgtggcctgtgagctgggcttctacaagtcagcccctggggaccagctgtgtgcccgctgccctccccacagccactccgcagctccagccgcccaagcctgccactgtgacctcagctactaccgtgcagccctggacccgccgtcctcagcctgcacccggccaccctcggcaccagtgaacctgatctccagtgtgaatgggacatcagtgactctggagtgggcccctcccctggacccaggtggccgcagtgacatcacctacaatgccgtgtgccgccgctgcccctgggcactgagccgctgcgaggcatgtgggagcggcacccgctttgtgccccagcagacaagcctggtgcaggccagcctgctggtggccaacctgctggcccacatgaactactccttctggatcgaggccgtcaatggcgtgtccgacctgagccccgagccccgccgggccgctgtggtcaacatcaccacgaaccaggcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtgatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctga

SEQ ID NO: 2 - protein, 1005 aa, Homo sapiens
MAPARGRLPPALWVVTAAAAAATCVSAARGEVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 3 - DNA, 1545 bp, Homo sapiens
  CDS (1)..(1545)
gcgcgcggcgaagtgaatttgctggacacgtcgaccatccacggggactggggctggctcacgtatccggctcatgggtgggactccatcaacgaggtggacgagtccttccagcccatccacacgtaccaggtttgcaacgtcatgagccccaaccagaacaactggctgcgcacgagctgggtcccccgagacggcgcccggcgcgtctatgctgagatcaagtttaccctgcgcgactgcaacagcatgcctggtgtgctgggcacctgcaaggagaccttcaacctctactacctggagtcggaccgcgacctgggggccagcacacaagaaagccagttcctcaaaatcgacaccattgcggccgacgagagcttcacaggtgccgaccttggtgtgcggcgtctcaagctcaacacggaggtgcgcagtgtgggtcccctcagcaagcgcggcttctacctggccttccaggacataggtgcctgcctggccatcctctctctccgcatctactataagaagtgccctgccatggtgcgcaatctggctgccttctcggaggcagtgacgggggccgactcgtcctcactggtggaggtgaggggccagtgcgtgcggcactcagaggagcgggacacacccaagatgtactgcagcgcggagggcgagtggctcgtgcccatcggcaaatgcgtgtgcagtgccggctacgaggagcggcgggatgcctgtgtggcctgtgagctgggcttctacaagtcagcccctggggaccagctgtgtgcccgctgccctccccacagccactccgcagctccagccgcccaagcctgccactgtgacctcagctactaccgtgcagccctggacccgccgtcctcagcctgcacccggccaccctcggcaccagtgaacctgatctccagtgtgaatgggacatcagtgactctggagtgggcccctcccctggacccaggtggccgcagtgacatcacctacaatgccgtgtgccgccgctgcccctgggcactgagccgctgcgaggcatgtgggagcggcacccgctttgtgccccagcagacaagcctggtgcaggccagcctgctggtggccaacctgctggcccacatgaactactccttctggatcgaggccgtcaatggcgtgtccgacctgagccccgagccccgccgggccgctgtggtcaacatcaccacgaaccaggcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggacc

SEQ ID NO: 4 - protein, 515 aa, Homo sapiens
ARGEVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRT

SEQ ID NO: 5 - DNA, 1135 bp, Homo sapiens
  CDS (2)..(1123)
caccagatctatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgaaagcttggc

SEQ ID NO: 6 - protein, 374 aa, Homo sapiens
TRSIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 7 - DNA, 925 bp, Homo sapiens
  CDS (2)..(913)
caccagatctatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcgccctcatgggccaccagaagaagatcctgggcagcattcagaccatgcgggcccagctgaccagcacccaggggccccgccggcacctctgaaagcttggc

SEQ ID NO: 8 - protein, 304 aa, Homo sapiens
TRSIHIEKIIGSGDSGEVCYGRLRVPGQRDVPVAIKALKAGYTERQRRDFLSEASIMGQFDHPNIIRLEGVVTRGRLAMIVTEYMENGSLDTFLRGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGIALMGHQKKILGSIQTMRAQLTSTQGPRRHL

SEQ ID NO: 9 - DNA, 925 bp, Homo sapiens
  CDS (2)..(913)
caccagatctatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattagaccatcccaacatcatccgcctcgagggtgtcgtcacccgtggccgcctggcaatgattgtgactgagtacatggagaacggctctctggacaccttcctgaggggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgaaagcttggc

SEQ ID NO: 10 - protein, 304 aa, Homo sapiens
TRSIHIEKIIGSGDSGEVCYGRLRVPGQRDVPVAIKALKAGYTERQRRDFLSEASIMGQLDHPNIIRLEGVVTRGRLAMIVTEYMENGSLDTFLRGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 11 - DNA, 925 bp, Homo sapiens
  CDS (2)..(913)
caccagatctatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacgtctctctggacaccttcctgaggggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgaaagcttggc

SEQ ID NO: 12 - protein, 304 aa, Homo sapiens
TRSIHIEKIIGSGDSGEVCYGRLRVPGQRDVPVAIKALKAGYTERQRRDFLSEASIMGQFDHPNIIRLEGVVTRGRLAMIVTEYMENVSLDTFLRGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 13 - DNA, 925 bp, Homo sapiens
  CDS (2)..(913)
caccagatctatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgaaagcttggc

SEQ ID NO: 14 - protein, 304 aa, Homo sapiens
TRSIHIEKIIGSGDSGEVCYGRLRVPGQRDVPVAIKALKAGYTERQRRDFLSEASIMGQFDHPNIIRLEGVVTRGRLAMIVTEYMENGSLDTFLRGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 15 - DNA, 925 bp, Homo sapiens
  CDS (2)..(913)
caccagatctatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacgtctctctggacaccttcctgaggggcgggaagatccccatccgctggacggccccagaggccatcgccttccgcaccttctcctcggccagcgacgtgtggagcttcggcgtggtcatgtggggggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgaaagcttggc

SEQ ID NO: 16 - protein, 304 aa, Homo sapiens
TRSIHIEKIIGSGDSGEVCYGRLRVPGQRDVPVAIKALKAGYTERQRRDFLSEASIMGQFDHPNIIRLEGVVTRGRLAMIVTEYMENVSLDTFLRGGKIPIRWTAPEAIAFRTFSSASDVWSFGVVMWGVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 17 - DNA, 1762 bp, Homo sapiens
  CDS (2)..(1735)
aggctccgcggccgcccccttcaccagatctgcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgaaagcttggcaagggtgggcgcgcc

SEQ ID NO: 18 - protein, 578 aa, Homo sapiens
GSAAAPFTRSAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 19 - DNA, 1762 bp, Homo sapiens
  CDS (2)..(1735)
aggctccgcggccgcccccttcaccagatctgcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcgcaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgaaagcttggcaagggtgggcgcgcc

SEQ ID NO: 20 - protein, 578 aa, Homo sapiens
GSAAAPFTRSAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAQPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 21 - DNA, 1726 bp, Homo sapiens
  CDS (2)..(1714)
caccagatctgcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgaaagcttggc

SEQ ID NO: 22 - protein, 571 aa, Homo sapiens
TRSAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 23 - DNA, 1433 bp, Homo sapiens
  CDS (2)..(1432)
aggctccgcggccgcccccttcaccagatctgcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcaagcttggcaagggtgggcgcgccgacccagctttcttgtacaaag

SEQ ID NO: 24 - protein, 477 aa, Homo sapiens
GSAAAPFTRSAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVKLGKGGRADPAFLYK

SEQ ID NO: 25 - DNA, 1411 bp, Homo sapiens
  CDS (2)..(1411)
aggctccgcggccgcccccttcaccagatctgcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcgcaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcaagcttggcaagggtgggcgcgcc

SEQ ID NO: 26 - protein, 470 aa, Homo sapiens
GSAAAPFTRSAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAQPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVKLGKGGRA

SEQ ID NO: 27 - DNA, 1439 bp, Homo sapiens
  CDS (2)..(1438)
aggctccgcggccgcccccttcaccagatctgcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccgtcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcaagcttggcaagggtgggcgcgcgacccagcttcttgtacaagttggatata

SEQ ID NO: 28 - protein, 479 aa, Homo sapiens
GSAAAPFTRSAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAVMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVKLGKGGRATQLLVQVGY

SEQ ID NO: 29 - DNA, 1375 bp, Homo sapiens
  CDS (2)..(1375)
caccagatctgcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcaagcttggc

SEQ ID NO: 30 - protein, 458 aa, Homo sapiens
TRSAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVKLG

SEQ ID NO: 31 - DNA, 1545 bp, Homo sapiens; identical to SEQ ID NO: 3
  CDS (1)..(1545)

SEQ ID NO: 32 - protein, 515 aa, Homo sapiens; identical to SEQ ID NO: 4

SEQ ID NO: 33 - DNA, 2884 bp, Homo sapiens
  CDS (1)..(2805)
atggcccccgcccggggccgcctgccccctgcgctctgggtcgtcacggccgcggcggcggcggccacctgcgtgtccgcggcgcgcggcgaagtgaatttgctggacacgtcgaccatccacggggactggggctggctcacgtatccggctcatgggtgggactccatcaacgaggtggacgagtccttccagcccatccacacgtaccaggtttgcaatgtcatgagccccaaccagaacaactggctgcgcacgagctgggtcccccgagacggcgcccggcgcgtctatgctgagatcaagtttaccctgcgcgactgcaacagcatgcctggtgtgctgggcacctgcaaggagaccttcaacctctactacctggagtcggaccgcgacctgggggccagcacacaagaaagccagttcctcaaaatcgacaccattgcggccgacgagagcttcacaggtgccgaccttggtgtgcggcgtctcaagctcaacacggaggtgcgcagtgtgggtcccctcagcaagcgcggcttctacctggccttccaggacataggtgcctgcctggccatcctctctctccgcatctactataagaagtgccctgccatggtgcgcaatctggctgccttctcggaggcagtgacgggggccgactcgtcctcactggtggaggtgaggggccagtgcgtgcggcactcagaggagcgggacacacccaagatgtactgcagcgcggagggcgagtggctcgtgcccatcggcaaatgcgtgtgcagtgccggctacgaggagcggcgggatgcctgtgtggcctgtgagctgggcttctacaagtcagcccctggggaccagctgtgtgcccgctgccctccccacagccactccgcagctccagccgcccaagcctgccactgtgacctcagctactaccgtgcagccctggacccgccgtcctcagcctgcacccggccaccctcggcaccagtgaacctgatctccagtgtgaatgggacatcagtgactctggagtgggcccctcccctggacccaggtggccgcagtgacatcacctacaatgccgtgtgccgccgctgcccctgggcactgagccgctgcgaggcatgtgggagcggcacccgctttgtgccccagcagacaagcctggtgcaggccagcctgctggtggccaacctgctggcccacatgaactactccttctggatcgaggccgtcaatggcgtgtccgacctgagccccgagccccgccgggccgctgtagtcaacatcaccacgaaccaggcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcaccccagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgatgtacagccagcagggcccaggcagccaccgagcccaccccaggtcatgccagcggcagaggacgtgaggggctgg

SEQ ID NO: 34 - protein, 935 aa, Homo sapiens
MAPARGRLPPALWVVTAAAAAATCVSAARGEVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTPAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 35 - DNA, 2884 bp, Homo sapiens
  CDS (1)..(2805)
atggcccccgcccggggccgcctgccccctgcgctctgggtcgtcacggccgcggcggcggcggccacctgcgtgtccgcggcgcgcggcgaagtgaatttgctggacacgtcgaccatccacggggactggggctggctcacgtatccggctcatgggtgggactccatcaacgaggtggacgagtccttccagcccatccacacgtaccaggtttgcaatgtcatgagccccaaccagaacaactggctgcgcacgagctgggtcccccgagacggcgcccggcgcgtctatgctgagatcaagtttaccctgcgcgactgcaacagcatgcctggtgtgctgggcacctgcaaggagaccttcaacctctactacctggagtcggaccgcgacctgggggccagcacacaagaaagccagttcctcaaaatcgacaccattgcggccgacgagagcttcacaggtgccgaccttggtgtgcggcgtctcaagctcaacacggaggtgcgcagtgtgggtcccctcagcaagcgcggcttctacctggccttccaggacataggtgcctgcctggccatcctctctctccgcatctactataagaagtgccctgccatggtgcgcaatctggctgccttctcggaggcagtgacgggggccgactcgtcctcactggtggaggtgaggggccagtgcgtgcggcactcagaggagcgggacacacccaagatgtactgcagcgcggagggcgagtggctcgtgcccatcggcaaatgcgtgtgcagtgccggctacgaggagcggcgggatgcctgtgtggcctgtgagctgggcttctacaagtcagcccctggggaccagctgtgtgcccgctgccctccccacagccactccgcagctccagccgcccaagcctgccactgtgacctcagctactaccgtgcagccctggacccgccgtcctcagcctgcacccggccaccctcggcaccagtgaacctgatctccagtgtgaatgggacatcagtgactctggagtgggcccctcccctggacccaggtggccgcagtgacatcacctacaatgccgtgtgccgccgctgcccctgggcactgagccgctgcgaggcatgtgggagcggcacccgctttgtgccccagcagacaagcctggtgcaggccagcctgctggtggccaacctgctggcccacatgaactactccttctggatcgaggccgtcaatggcgtgtccgacctgagccccgagccccgccgggccgctgtagtcaacatcaccacgaaccaggcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgatgtacagccagcagggcccaggcagccaccgagcccaccccaggtcatgccagcggcagaggacgtgaggggctgg

SEQ ID NO: 36 - protein, 935 aa, Homo sapiens
MAPARGRLPPALWVVTAAAAAATCVSAARGEVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 37 - DNA, 22 bp, Artificial Sequence
  Description of Artifical Sequence  Primer/ Probe
gcacacaagaaagccagttcct

SEQ ID NO: 38 - DNA, 23 bp, Artificial Sequence
  Description of Artifical Sequence  Primer/ Probe
taaaatcgacaccattgcggccg

SEQ ID NO: 39 - DNA, 19 bp, Artificial Sequence
  Description of Artifical Sequence  Primer/ Probe
ggtcggcacctgtgaagct

SEQ ID NO: 40 - DNA, 18 bp, Artificial Sequence
  Description of Artifical Sequence  Primer/ Probe
gtgcggagagcgagggag

SEQ ID NO: 41 - DNA, 19 bp, Artificial Sequence
  Description of Artifical Sequence  Primer/ Probe
catgacctggggtgggctt